(19) United States
(12) Patent Application Publication
(10) Pub. No.: US 2026/0232711 A1
(43) Pub. Date: Aug. 13, 2026

(54) METHOD FOR MANAGING DISORDERS ASSOCIATED WITH DISRUPTED CIRCADIAN RHYTHM

(71) Applicant: ONE BIO INC, Sacramento, CA (US)

(72) Inventors: Angela Maria MARCOBAL-BARRANCO, Sacramento, CA (US); Matthew Joseph AMICUCCI, Sacramento, CA (US); Bruce Robert McCONNELL, Sacramento, CA (US); Maria Ximena MALDONADO GÓMEZ, Sacramento, CA (US); Katharine NG, Sacramento, CA (US); Nithya KRISHNAKUMAR, Sacramento, CA (US); Steven Michael WATKINS, Sacramento, CA (US); Alexandria Marie Salazar CONNER, Sacramento, CA (US)

(73) Assignee: ONE BIO INC, Sacramento,, CA (US)

(21) Appl. No.: 19/154,721

(22) PCT Filed: Feb. 27, 2024
(86) PCT No.: PCT/US2024/017482

§ 371 Date: Aug. 7, 2025

Related U.S. Application Data
(60) Provisional application No. 63/448,956, filed on Feb. 28, 2023.

(51) Int. Cl. A61K 31/702; A61P 29/00

(52) U.S. Cl. CPC A61K 31/702; A61P 29/00

(57) ABSTRACT

Method of managing a disorder associated with disrupted circadian rhythm in a subject by administering to a subject an effective amount of a beta glucan oligosaccharide and/or an arabinan oligosaccharide. More specifically, management includes treating symptoms associated with disrupted circadian rhythm, primary prevention of symptoms associated with disrupted circadian rhythm, secondary prevention of symptoms associated with disrupted circadian rhythm and/or delaying the progression of symptoms associated with disrupted circadian rhythm. Symptoms to be managed include among others sleep-related symptoms, mood-related symptoms, and symptoms associated with cognitive functioning and neurocognitive decline. In a particular method herein, the subject is assessed for risk of developing symptoms associated with disrupted circadian rhythm prior to administering the beta glucan oligosaccharide and/or arabinan oligosaccharide. Medicaments, nutritional formulations, nutritional supplements and pharmaceutical compositions comprising Nan effective amount of a beta glucan oligosaccharide and/or an arabinan oligosaccharide for such treatment, prevention and delay are also provided.

### CROSS-REFERENCE TO RELATED APPLICATIONS

This application claims the benefit of priority to U.S. Provisional Patent Application No. 63/448,956, filed Feb. 28, 2023, which is hereby incorporated by reference in its entirety.

### FIELD OF THE INVENTION

This invention relates to methods for managing disorders associated with disrupted circadian rhythm.

### BACKGROUND OF THE INVENTION

Circadian rhythms synchronize important physiological activities at every level of organization in the body, from sleep-wake timing to cellular-level metabolic processes. Sleep in particular is regulated by circadian rhythms that control the sleep-wake cycle which aligns sleep and wakefulness with day and night. Disruption of the circadian clock is closely related to the development of sleep disorders.

Adequate, quality sleep is critical for health and functioning. For healthy adults, the recommended minimum amount of sleep is 7 hours per night. However, many people do not reach this minimum amount of sleep. In some cases, this is due to temporary sleep-related difficulties related to stress or life circumstances. In other cases, the situation is chronic and sleep disorders regularly reduce the amount and quality of sleep.

In general, sleep disorders are characterized as chronic sleep conditions that impact quality of life or ability to function. Common sleep disorders include insomnia and sleep-wake disorders. Insomnia is defined as having difficulty falling or staying asleep. Short-term insomnia occurs in nearly 30% of U.S. adults, and chronic insomnia occurs in 10% of U.S. adults. Sleep-wake disorders or circadian rhythm sleep disorders involve alterations in a person's normal 24-hour biological clock. A person may be suffering from a sleep-wake disorder if they have problems with either sleep initiation, frequent night awakenings, early awakenings, or poor sleep quality.

It is estimated that the global prevalence of sleep disorders in the general population ranges from about 20% to about 42%. In the United States, it is estimated that about 50 to 70 million adults are impacted by lack of sleep. These disorders commonly lead to health conditions such as anxiety, depression, irritability, diabetes, heart disease and decreased immunity. For example, about 40% of people with insomnia are believed to be affected by a mental health disorder while about 75% of adults with depression suffer from insomnia.

Further, more than 90% of people with Post Trauma Stress Disorder (PTSD) related to military combat have symptoms of insomnia. Lack of sleep has a consequential economic impact which is large. For example, insufficient sleep has an estimated economic impact of over US$411 Billion each year in the United States.

In general, chronic lack of sleep is treated using prescription sleep medications. However, these medications generally have side effects. For example, about 80% of people in the US who take prescription sleep medications experience residual effects like oversleeping, feeling groggy, or having a hard time concentrating the next day. To avoid these side effects, many people try natural remedies for sleep problems, such as melatonin supplements. The National Center for Complementary and Integrative Health suggests that melatonin supplements may help with certain short-term sleep issues. However, the American Academy of Sleep Medicine and the American College of Physicians, indicate that there is not enough strong evidence on the effectiveness or safety of melatonin supplementation for chronic insomnia to recommend its use. Instead, the American College of Physicians guidelines strongly recommend the use of cognitive behavioral therapy for insomnia (CBT-I) as an initial treatment for insomnia.

However, chronic breakdown of circadian rhythms is a significant risk factor for a range of diseases, including psychiatric disturbances, and neurodegenerative diseases. For example, depression is closely related to circadian activity (Teichman et al, 2020). Depressed patients often experience milder symptoms at night and more severe symptoms in the morning. The incidence of depressive symptoms among shift workers is significantly higher than that among normal workers. In addition, the clinical manifestations of seasonal affective disorder are depressive symptoms related to specific seasons, while the onset of seasonal affective disorder is related to biological rhythms such as day length and the intensity of ambient light. This indicates a close relationship between depression and biological rhythms. Also, depressive episodes are also often associated with sleep disorders. For example, depressive patients usually have decreased sleep time, increased rapid eye movement (REM) sleep or shorter REM latency.

Depression is one of several related mood disorders. Other disorders of mood include major depressive disorder (MDD or clinical depression), dysthymia, and bipolar disorder. Patients suffering from depression exhibit feelings of sadness, low mood and an aversion to activity and this mood can affect a person's thoughts, behavior, feelings and sense of well-being. A depressed person may feel sad, anxious, empty, hopeless, worried, helpless, worthless, guilty, irritable, hurt, or restless.

Many types of antidepressant medications are available to treat mood disorders that present with depression. Some available drugs include selective serotonin reuptake inhibitors (SSRIs), serotonin and norepinephrine reuptake inhibitors (SNRIs), norepinephrine and dopamine reuptake inhibitors (NDRIs), tricyclic antidepressants, monoamine oxidase inhibitors (MAOIs), and atypical antidepressants such as vortioxetine. However, despite the availability of numerous treatment options, individual response to antidepressant medication is suboptimal and variable. As many as half of patients do not receive adequate treatment and many respond partially or not at all to treatment. The presence of residual symptoms is also associated with a higher risk of recurrence, more chronic depressive episodes, and a shorter duration between episodes. Guidelines for treatment recommend four possible strategies for managing non-response or partial response including increasing the dose of the antidepressant drug, replacing the drug with a different antidepressant drug, augmenting the antidepressant therapy with a non-antidepressant agent, or combining the initial antidepressant with a second antidepressant. Despite the lack of general efficacy, all the available drugs have side effects with many having serious side effects.

Neurodegenerative diseases such as Alzheimer's disease and Parkinson's disease also appear to be closely related to chronic breakdown of circadian rhythms and sleep disorders. Sleep disruption usually precedes clinical diagnoses of neurodegenerative diseases by several years. Also, recent studies have revealed robust association between weakened rest/activity rhythmicity and the subsequent development of Alzheimer's disease and Parkinson's disease years later (Fifel et al., 2021). Stabilizing circadian rhythms may therefore be a useful approach to delay or prevent neurodegenerative diseases. At the present time, there are no approved treatments targeting the causes of neurodegenerative diseases, only treatments targeting symptoms.

In recent years, studies have suggested that the microbiota-gut-brain axis may provide a basis for explaining the link between circadian rhythm, sleep, mood, and neurodegenerative diseases. Recent evidence points to the microbiota as both being modulated by, and a modulator of, the central and peripheral clocks (Teichman et al, 2020). The gut microbiota impacts brain function through 3 main pathways that produce a bidirectional flow of information.

The first of these is the immunoregulatory pathway, in which the microbiota interact with immune cells in such a way as to affect the levels of cytokines and prostaglandin E2. For example, bacterial lipopolysaccharide, which is a pro-inflammatory neurotoxin, may be able to cross the epithelial gut barrier when disrupted and enter systemic circulation. The lipopolysaccharide may then access the hippocampus, a brain region associated with the control of learning and memory processes, leading to cognitive impairments and neuroinflammation.

The second is the neuroendocrine pathway. The intestine constitutes the largest endocrine organ in the human body. The gut microbiota may impact the hypothalamic-pituitary-adrenal (HPA) axis and the central nervous system by regulating the secretion of neurotransmitters such as cortisol, tryptophan, dopamine, noradrenaline and serotonin.

The third is the vagus nerve pathway in combination with the enteric nervous system. Anatomical evidence indicates that the sensory neurons of the intestinal myenteric plexus are exposed to the gut microbiota. These sensory neurons form synaptic contacts with motor neurons in the intestine that are involved in the regulation of intestinal motility and gut hormone secretion. The enteric nervous system also forms synaptic connections with the vagus nerve, which connects the intestine to the brain and constitutes an information transmission pathway. Neurotoxic products and metabolites such as D-lactic acid and ammonia produced by the gut microbiota may pass through the vagus nerve into the central nervous system, thereby affecting brain function, stress responses, and sleep structure.

The gut barrier function may play a role in linking these pathways and disorders associated with disrupted circadian rhythm. A disrupted gut barrier function, or “leaky gut”, results in transport of gut luminal contents to the lamina propria and activation of the immune system. This will trigger the pathways described above.

However, there are currently no approved methods for modulating the microbiota-gut-brain axis to manage disorders associated with disrupted circadian rhythm. Therefore, there remains a need for methods of managing disorders associated with disrupted circadian rhythm, especially methods with limited side effects.

### SUMMARY OF THE INVENTION

In one aspect, this invention provides a method of managing a disorder associated with disrupted circadian rhythm in a subject, the method comprising, consisting essentially of or consisting of administering to the subject an effective amount of a beta glucan oligosaccharide and/or an arabinan oligosaccharide.

In another aspect, this invention provides a method of treating symptoms associated with disrupted circadian rhythm in a subject, the method comprising, consisting essentially of or consisting of administering to the subject an effective amount of a beta glucan oligosaccharide and/or an arabinan oligosaccharide.

In a further aspect, this invention provides a method for the primary prevention of symptoms associated with disrupted circadian rhythm in a subject, the method comprising, consisting essentially of or consisting of administering to the subject an effective amount of a beta glucan oligosaccharide and/or an arabinan oligosaccharide. In an embodiment, the method further comprises, consists essentially of or consists of determining whether the subject is at risk of developing symptoms associated with disrupted circadian rhythm, prior to administering the beta glucan oligosaccharide and/or arabinan oligosaccharide. In an embodiment, the intestinal barrier function of the subject is assessed to determine whether the subject is at risk.

In an embodiment, the intestinal barrier function of the subject is assessed using one or more of lactulose and mannitol permeability, blood zonulin levels, dynamic contrast-enhanced magnetic resonance imaging, histology and confocal laser endomicroscopy.

In a yet further aspect, this invention provides method for the secondary prevention of symptoms associated with disrupted circadian rhythm in a subject, the method comprising, consisting essentially of or consisting of administering to the subject an effective amount of a beta glucan oligosaccharide and/or an arabinan oligosaccharide.

In another aspect, this invention provides a method of delaying the progression of symptoms associated with disrupted circadian rhythm in a subject, the method comprising, consisting essentially of or consisting of administering to the subject an effective amount of a beta glucan oligosaccharide and/or an arabinan oligosaccharide.

In an embodiment, the symptoms for all aspects are sleep-related symptoms. In an embodiment, the symptoms for all aspects are mood-related symptoms. In an embodiment, the symptoms for all aspects are symptoms associated with cognitive functioning decline and/or neurocognitive decline. In embodiments, symptoms for all aspects are a combination of sleep-related symptoms, and/or mood-related symptoms, and/or symptoms associated with cognitive functioning decline and/or neurocognitive decline.

In an embodiment, the subject is administered an amount of the oligosaccharide in the range from about 500 mg to about 50 g per day, in certain embodiments from about 1 g to about 20 g per day, for example from about 2 g to about 10 g per day, preferably about 3 g to about 6 g per day.

In an embodiment, the subject is administered an amount of the oligosaccharide for a period of at least about 14 days, in certain embodiments for at least about 1 month, for example for at least about 6 months. The subject can be administered an amount of the oligosaccharide for a period of at least about 1 year, or chronically for the rest of the adult's life.

The invention also provides the use of a beta glucan oligosaccharide, an arabinan oligosaccharide or a combination thereof, as described herein, for managing a disorder or managing symptoms associated with disruption of circadian rhythm. In another aspect, the invention provides the use of a beta glucan oligosaccharide, an arabinan oligosaccharide or a combination thereof, as described herein, for treating symptoms associated with disrupted circadian rhythm in a subject. In a further aspect, the invention provides the use of a beta glucan oligosaccharide, an arabinan oligosaccharide or a combination thereof, as described herein, for the primary prevention of symptoms associated with disrupted circadian rhythm in a subject. In an embodiment, the use optionally further comprises determining whether the subject is at risk of developing symptoms associated with disrupted circadian rhythm, prior to using the beta glucan oligosaccharide and/or arabinan oligosaccharide. In an embodiment, the intestinal barrier function of the subject is assessed as described herein to determine whether the subject is at risk. In a yet further aspect, the invention provides the use of a beta glucan oligosaccharide, an arabinan oligosaccharide or a combination thereof for the secondary prevention of symptoms associated with disrupted circadian rhythm in a subject. In another aspect, the invention provides the use of a beta glucan oligosaccharide, an arabinan oligosaccharide or a combination thereof, as described herein, for delaying the progression of symptoms associated with disrupted circadian rhythm in a subject. The uses of the beta glucan oligosaccharide, the arabinan oligosaccharide or the combination thereof, as described herein, involve administering to the subject an effective amount of a beta glucan oligosaccharide and/or an arabinan oligosaccharide for achieving the desired management, treatment or prevention. In embodiments, the effective amount of the beta glucan oligosaccharide, the arabinan oligosaccharide or the combination thereof is administered in a nutritional formula, a nutritional supplement, or pharmaceutical composition or added to a food item, beverage or drink.

In additional aspects, the invention provides use of a beta glucan oligosaccharide, an arabinan oligosaccharide or a combination thereof, as described herein, for the preparation of a medicament for management or treatment of a disorder, or one or more symptoms associated with a disruption of circadian rhythm. The medicament includes among others a nutritional formula, a nutritional supplement, or a pharmaceutical composition. In another aspect, the invention provides the use of a beta glucan oligosaccharide, an arabinan oligosaccharide or a combination thereof, as described herein, for the preparation of a medicament for treating symptoms associated with disrupted circadian rhythm in a subject. In further aspects, the invention provides the use of a beta glucan oligosaccharide, an arabinan oligosaccharide or a combination thereof, as described herein, for preparation of a medicament for primary prevention of symptoms associated with disrupted circadian rhythm in a subject. In an embodiment, the use of such medicament optionally further comprises determining whether the subject is at risk of developing symptoms associated with disrupted circadian rhythm, prior to using the beta glucan oligosaccharide and/or arabinan oligosaccharide medicament. In an embodiment, the intestinal barrier function of the subject is assessed as described herein to determine whether the subject is at risk. In a yet further aspect, the invention provides the use of a beta glucan oligosaccharide, an arabinan oligosaccharide or a combination thereof, as described herein, for the production of a medicament for secondary prevention of symptoms associated with disrupted circadian rhythm in a subject. In another aspect, the invention provides the use of a beta glucan oligosaccharide, an arabinan oligosaccharide or a combination thereof, as described herein, for the production of a medicament for delaying the progression of symptoms associated with disrupted circadian rhythm in a subject. The preparation of the medicaments involves preparation of an effective amount of a beta glucan oligosaccharide, an arabinan oligosaccharide or a combination thereof optionally in combination with a nutritionally or pharmaceutically acceptable excipient or carrier. The use of the beta glucan oligosaccharide and/or the arabinan oligosaccharide medicament prepared involves administering to the subject of an effective amount of a beta glucan oligosaccharide and/or an arabinan oligosaccharide for achieving the desired management, treatment or prevention.

In embodiments, beta glucan oligosaccharide and arabinan oligosaccharide for use in the methods, formulations, supplements, and compostions herein are prepared by Fenton-type depolymerizations as described in any of WO2021097138A1, WO2018236917A1. WO2020247389A1, WO2022241163A1 and WO2023220318.

In embodiments, the symptoms for all aspects are sleep-related symptoms. In embodiments, the symptoms for all aspects are mood-related symptoms. In an embodiment, the symptoms for all aspects are symptoms associated with cognitive functioning decline and/or neurocognitive decline. In embodiments, symptoms for all aspects are a combination of sleep-related symptoms, and/or mood-related symptoms, and/or symptoms associated with cognitive functioning decline and/or neurocognitive decline.

In embodiments of all forgoing aspects, the subject whose symptoms are managed is a patient who has been diagnosed with a disorder associated with disruption of circadian rhythm. In an embodiment, the subject is a patient who has been diagnosed with a sleep-related disorder. In an embodiment, the subject is a patient who has been diagnosed with a mood-related disorder. In an embodiment, the subject is a patient who has been diagnosed with cognitive functioning decline. In an embodiment, the subject is a patient who has been diagnosed with neurocognitive decline.

In embodiments of all forgoing aspects, the beta glucan oligosaccharide and/or the arabinan oligosaccharide is selected from the group consisting of CLX115, CLX122, CLX115Cu, CLX122DSF, CLX112, or any combination thereof. In embodiments of all foregoing aspects, the beta glucan oligosaccharide is CLX115Cu.

The invention further relates to a beta glucan oligosaccharide, an arabinan oligosaccharide, or a combination thereof for the management, primary prevention, secondary prevention, or treatment of a disorder and/or symptom associated with disrupted circadian rhythm.

The invention further relates to nutritional formulations, nutritional supplements, pharmaceutical compostions and medicaments which comprise, consist essentially of, or consist of an effective amount of a beta glucan oligosaccharide, an arabinan oligosaccharide or a combination thereof as described herein for the management, primary prevention, secondary prevention or treatment of a disorder and/or symptom associated with disrupted circadian rhythm.

Other aspects and embodiments of the disclosure will be apparent to one of ordinary skill in the art in view of the descriptions, drawings and non-limited examples herein.

### BRIEF DESCRIPTION OF THE DRAWINGS

FIGS. 1A and 1B show the production of butyrate, lactate, and propionate after 20 hours static fecal fermentation, using fecal samples from donor A (FIG. 1A) and donor B (FIG. 1B).

FIGS. 2A and 2B show the effect of donor A (FIG. 2A) and donor B (FIG. 2B) fecal fermentation supernatants on Caco-2 cell epithelial barrier integrity when challenged with 10 ng/mL TNF-α and IFN-γ. The dashed Na-Bu line represents the average % change in transepithelial electrical resistance (TEER) after treatment with 5 mM sodium butyrate positive control. Dashed control line represents the average % change in TEER after treatment with minimum essential medium (MEM) only.

FIGS. 3A and 3B show the effect of SHIME fecal fermentation supernatants from proximal and distal colon vessels on Caco-2 cell epithelial barrier integrity. Untreated supernatants correspond to the control phase of the SHIME experiment in proximal colon (FIG. 3A) and distal colon (FIG. 3B).

FIGS. 4A-4C show the effect of SHIME fecal fermentation supernatants from distal colon vessel suspensions, at control and treatment phase, using oligosaccharide CLX115, on secretion of pro-inflammatory chemokines (CXCL10, IL-8 and MCP-1) (FIG. 4A), anti-inflammatory cytokines (IL-10 and IL-6) (FIG. 4B) and pro-inflammatory cytokines (IL-1β and TNF-α) (FIG. 4C).

FIGS. 5A-5C show the effect of SHIME fecal fermentation supernatants from proximal colon vessel suspensions, at control and treatment phase, using oligosaccharide CLX115, on secretion of pro-inflammatory chemokines (CXCL10, IL-8 and MCP-1) (FIG. 5A), anti-inflammatory cytokines (IL-10 and IL-6) (FIG. 5B), and pro-inflammatory cytokines (IL-1β and TNF-α) (FIG. 5C).

FIGS. 6A-6C show the effect of SHIME fecal fermentation supernatants from distal colon vessel suspensions, at control and treatment phase, using oligosaccharide CLX122, on secretion of pro-inflammatory chemokines (CXCL10, IL-8 and MCP-1) (FIG. 6A), anti-inflammatory cytokines (IL-10 and IL-6) (FIG. 6B), and pro-inflammatory cytokines (IL-1β and TNF-α) (FIG. 6C).

FIGS. 7A-7C shows the effect of SHIME fecal fermentation supernatants from proximal colon vessel suspensions, at control and treatment phase, using oligosaccharide CLX122, on secretion of pro-inflammatory chemokines (CXCL10, IL-8 and MCP-1) (FIG. 7A), anti-inflammatory cytokines (IL-10 and IL-6) (FIG. 7B), and pro-inflammatory cytokines (IL-1β and TNF-α) (FIG. 7C).

FIGS. 8A and 8B show glutamate and GABA levels after in vitro fecal fermentations with oligosaccharide CLX115 (FIG. 8A) and oligosaccharide CLX122 (FIG. 8B), performed with 20 different fecal samples from healthy donors.

FIG. 9 shows ammonia levels after in vitro fermentations with oligosaccharide CLX115, performed with 5 different fecal samples from healthy donors.

FIGS. 10A-10D are graphs of size exclusion chromatography coupled to refractive index molecular weight distribution of select oligosaccharides. The graph shows Abundance (Refractive Index Units (nRIU) as a function of elusion time (minutes). FIGS. 10A-10D illustrate molecular weight distributions of oligosaccharides, CLX115, CLX115Cu, CLX112 and CLX122DSF, respectively. Dashed vertical lines with annotated values denote molecular weight ranges (Daltons). Peaks are annotated with elusion time.

### DETAILED DESCRIPTION OF THE INVENTION

It has been surprisingly found that beta glucan oligosaccharides and/or arabinan oligosaccharides have a significant effect on improving and/or slowing the worsening of symptoms associated with disrupted circadian rhythm. Without being bound by hypothesis, it is believed that the oligosaccharides improve the intestinal barrier function, reducing permeability and improving barrier immune tone. This can beneficially impact immunomodulatory pathways linked to cognitive impairments and neuroinflammation. Moreover, it is believed that the oligosaccharides increase the production of the neurotransmitter GABA by the intestinal microbiota, which beneficially impacts hypothalamic-pituitary-adrenal (HPA) axis. Further, the oligosaccharides can reduce ammonia levels in the intestine which reduces the negative impact ammonia may have on the vagus nerve pathway.

In this specification, the following terms have the following meaning unless otherwise specified:

“About” or “approximately” mean being close to the value or range following these terms, as understood by one of ordinary skill in the art and include a deviation up to ±10% of the values or ranges that follow.

“Adult” means a human of age over 18 years.

“Adult nutritional formula” means a foodstuff which is intended to satisfy the particular nutritional needs of adults, particularly elderly adults. The adult nutritional formula may be a complete nutritional formula which satisfies all the nutritional needs of the adult or a supplement to diet. The adult nutritional formulas are often regulated as a food for special medical purposes/medical food, but this may vary from country to country. However, the adult nutritional formula can fall within other food types such as dietary supplements, and foods.

“Ammonium bicarbonate” means solid ammonium bicarbonate, and/or an aqueous solution containing: ammonium and bicarbonate; ammonium, OH⁻, and CO₂; ammonia, H₂O, and CO₂; or any of the preceding and their equilibrium products.

“Ammonium hydroxide” means aqueous ammonium hydroxide and/or a solution containing: ammonia and H₂O; ammonium and OH⁻; ammonia and OH⁻; or any of the preceding and their equilibrium products.

“Arabinan oligosaccharide” means an oligosaccharide that contains alpha-linked arabinan residues and that optionally resembles a legume arabinan, such as, a pea arabinan, and/or a soy arabinan (i.e., arabinans from legume plant sources). Arabinan oligosaccharide can include arabinan from non-legume sources, such as beetroot arabinanan. The oligosaccharide can comprise alpha 1-5, alpha 1-3, or alpha 1-2 glycosidic linkages. Arabinan oligosaccharides can be linear or branched. In embodiments, the molecular weight distribution of arabinan oligosaccharides is such that at least 50% of the mass is lower in molecular weight than 50 kDa. Arabinan oligosaccharides can be created through enzymatic, chemical, or biological synthesis or through the depolymerization of arabinan via enzymatic, chemical, physical, or biological processes. Arabinan oligosaccharides can be made through Fenton-type depolymerizations as described in WO2021097138A1, WO2018236917A1, WO2020247389A1, and WO2022241163A1, which are each incorporated by reference herein it its entirety to the extent not inconsistent with the description herein. The references are incorporated by reference herein for any purpose and particularly for descriptions of Fenton-type depolymerizations. Oligosaccharides CLX122, and CLX122DSF are all examples of arabinan oligosaccharides.

In embodiments herein glycosidic linkages, particularly those in arabinan oligosaccharides, are described as trisecting and in particular as trisecting in the 2, 3 and 5 position. Glycosidic linkages representing terminal, linear, bisecting, and trisecting monomers of glucose, galatose, mannose, xylose, arabinose, ribose, fucose, rhamnose, glucuronic acid and galacturonic acid are known in the art. [Galermo et al., 2018; Galermo et al., 2019]. Methods for characterization of glucosidic linkages, particularly with respect to bisecting or trisecting linkages are provided, for example, in Galermo et al., 2018, Galermo et al., 2019 and Amicucci et al., 2019 (Analytical Chemistry), each of which is incorporated by reference herein in its entirety for any purpose, but particularly for methods of characterizing glycosidic linkages in general, and more specifically for characterizing glycosidic linkages as bisecting or trisecting.

“Base” means a compound or collection of compounds that can accept hydrogen ions from a peroxyl oxidized carbohydrate, water, or non-aqueous solvent. Base can include Lewis bases, non-Arrhenius bases, weak-Arrhenius bases, other molecules that produce hydroxide ions through their decomposition, or other compounds that can accept hydrogen ions from a hydroperoxyl oxidized carbohydrate. Unless otherwise specified, base does not mean a strong-Arrhenius base (e.g., Na⁺OH⁻, KOH⁻, or Ca⁺²(OH⁻)₂).

“Beta glucan” means a polysaccharide that contains β-linked glucose residues. Beta glucan includes cereal beta glucans, yeast beta glucans, and fungal beta glucans. Beta glucan polymers can comprise beta 1-3, beta 1-4, or beta 1-6 glycosidic linkages. Beta glucans can be linear or branched. Within each class of beta glucans, the distribution of polymers is such that at least 80% of the mass is larger than 50 kda. “Beta glucan” can refer to the solid material after roasting, fermentation, hot-water, enzymatic, chemical, alkaline, super critical fluid, sun drying, organic solvent, acidic, mechanical pressure or pressure based extractions.

“Beta glucan oligosaccharide” means an oligosaccharide that contains beta-linked glucose residues and that resembles a cereal beta glucan, a yeast beta glucan, and/or a fungal beta glucan. The oligosaccharide can comprise beta 1-3, beta 1-4, and/or beta 1-6 glycosidic linkages. Beta glucan oligosaccharides can be linear or branched. The molecular weight distribution of beta glucan oligosaccharides is such that at least 50% of the mass is smaller than 5 kda. Beta glucan oligosaccharides can be created through enzymatic, chemical, or biological synthesis or through the depolymerization of beta glucans via enzymatic, chemical, physical, or biological processes. Beta glucan oligosaccharides can be made through Fenton-type depolymerizations as described in WO2021097138A1, WO2018236917A1, WO2020247389A1, and WO2022241163A1, which are each incorporated by reference herein it its entirety to the extent not inconsistent with the description herein. Oligosaccharides CLX112, CLX115, and CLX115Cu are all examples of beta glucan oligosaccharides.

“Bronsted-Lowry base” means a compound or atom that can accept or bond to a hydrogen ion (e.g., methanol, formaldehyde, ammonia, etc.).

“Cereal beta glucan” means a beta glucan found in the cell walls of cereals and which contains beta-linked glucose units that are in the beta-3 position and beta-4 positions. In cereals, the cereal beta glucan may be found alongside other polymers such as cellulose, starch, and arabinoxylans. In an embodiment, cereal beta glucan can have a structure in which the linear polymer is comprised of beta-4 linked glucose residues with beta-3 linked residues interspersed at a ratio of about 3:1 to 5:1 beta-4:beta-3 linked glucose residues. Cereal beta glucan can be obtained from cereals and grains such as oats, barley, wheat, rye, and rice, for example. Cereal beta glucans can be extracted from the bran or the endosperm of cereals and grains, and can be the solid material after roasting, fermentation, hot-water, enzymatic, chemical, alkaline, super critical fluid, sun drying, organic solvent, acidic, mechanical pressure or pressure-based extractions. The distribution of polymers in cereal beta glucan is such that at least 80% of the mass is larger than 50 kda.

“Cereal beta glucan oligosaccharide” means an oligosaccharide that resembles beta glucan found in the cell walls of cereals and contains beta-linked glucose units that are in the beta-3 position and beta-4 positions. Cereal beta glucan oligosaccharides may be found alongside polysaccharides or oligosaccharides such as cellulose, starch, and arabinoxylans in their polysaccharide or oligosaccharide forms. Cereal beta glucan oligosaccharides can have a structure in which the linear polymer is comprised of beta-4 linked glucose residues with beta-3 linked residues interspersed at a ratio of about 3:1 to 5:1 beta-4:beta-3 linked glucose residues. Cereal beta glucan oligosaccharides can be derived from cereal beta glucan. The molecular weight distribution of cereal beta glucan oligosaccharides is such that at least 50% of the mass is smaller than 5 kda. Cereal beta glucan oligosaccharides can refer to oligosaccharides created through enzymatic, chemical, or biological synthesis or through the depolymerization of beta glucans via enzymatic, chemical, physical, or biological processes. Cereal beta glucan oligosaccharides may be made through Fenton-type depolymerizations as described in WO2021097138A1, WO2018236917A1, WO2020247389A1. WO2022241163A1, which are each incorporated by reference herein in its entirety to the extent not inconsistent with the description herein. Oligosaccharides CLX112, CLX115, and CLX115Cu are all examples of “cereal beta glucan oligosaccharides”.

“Cleavage agent” or “cleavage reagent” means a single or collection of non-Arrhenius and/or weak-Arrhenius bases used to cleave polysaccharides after hydroperoxyl oxidation thereof. In certain aspects, a cleavage agent or cleavage reagent breaks glycosidic bonds in the polysaccharide, which bonds may be present between any two saccharides of the polysaccharide. The cleavage reagent may also be, and preferably is, a peroxide quenching reagent, and in either case may be used in combination with an additional compatible peroxide-quenching agent that may or may not also be a cleavage agent. In some aspects, a cleavage reagent may be an enzyme, for example a glycosyl hydrolase, a lytic polysaccharide monooxygenase, a glycosyl transferase, transglycosidase, polysaccharide lyase, carbohydrate binding module, glycoysl transferase, carbohydrate esterase, a cocktail containing two or more of the forementioned enzymes, or any enzyme that is carbohydrate active. In some aspects, a cleavage reagent may be a solid-phase acid catalyst or a solid-phase base catalyst.

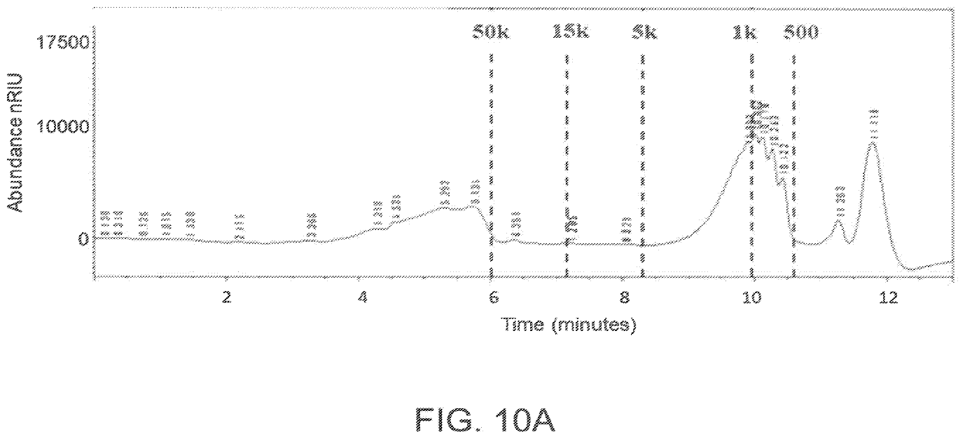

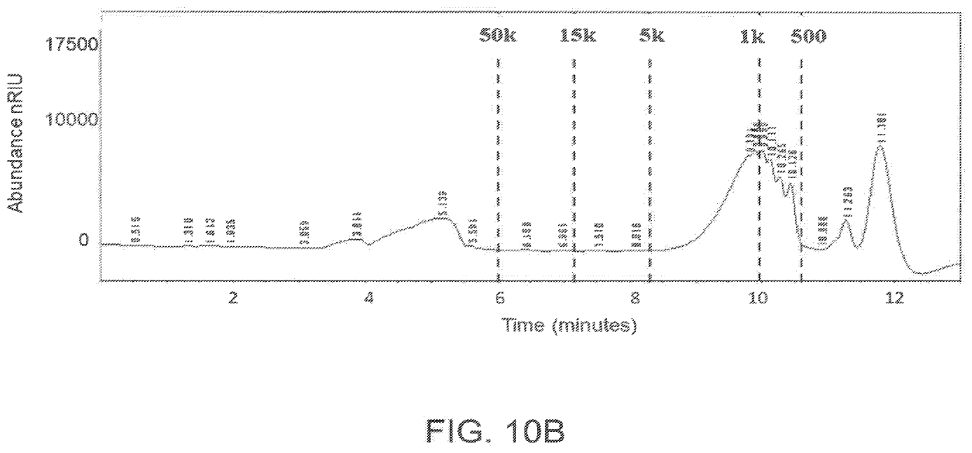

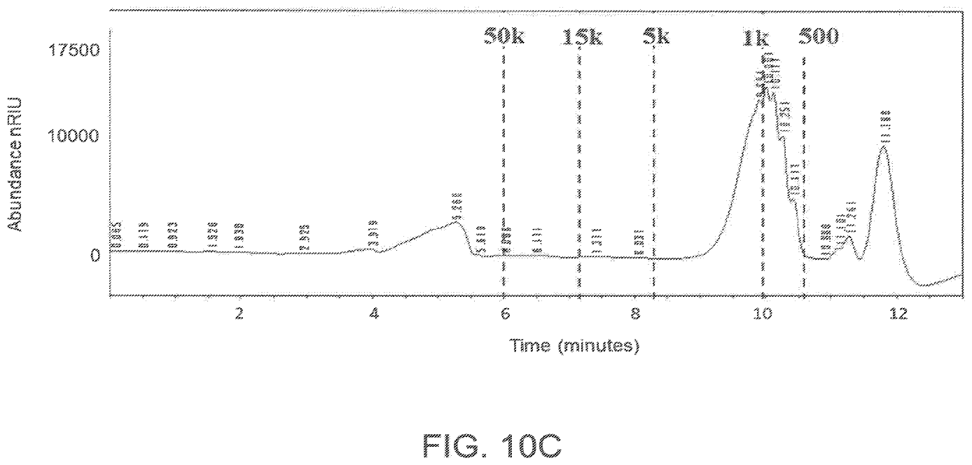

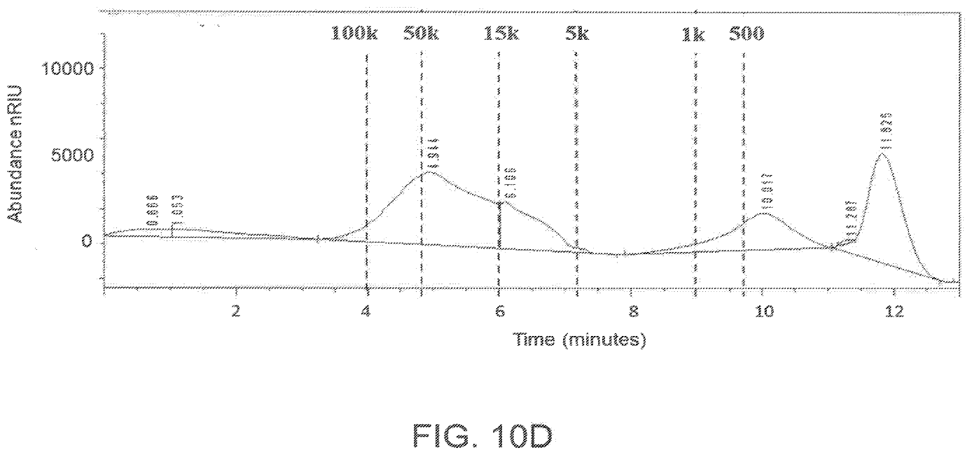

As used herein, the term “CLX115” refers to an oligosaccharide composition wherein about 95% of the mass comprises glucose and about 2% of the mass comprises arabinose, as measured by hydrolytic monosaccharide compositional analysis. For composition CLX115, the glycosidic linkage composition comprises, approximately, the amount set forth in Table B, for CLX115. The CLX115 composition comprises, approximately, the ¹H-¹³C HSQC NMR correlations set forth in Table A for CLX115. The CLX115 composition comprises, approximately, the values set forth in Table D, as measured by oligosaccharide analysis. The molecular weight distribution of CLX115 composition comprises, approximately, the values set forth in Table H, as measured by refractive index detection (RID) (see also FIG. 10A). CLX115 has a dynamic viscosity of about 1.382 mPa s at 100 mg/ml at 25° C. CLX 115 generally is derived from oat beta glucan, but can also be derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, and oligosaccharide analysis as CLX115. In an embodiment, CLX 115 was produced from oat beta glucan in accordance with the depolymerization described in Example 8.

As used herein, the term “CLX112” refers to an oligosaccharide composition wherein about 97% of the mass comprises glucose, as measured by hydrolytic monosaccharide compositional analysis. For composition CLX112, the glycosidic linkage composition comprises, approximately, the amount set forth in Table B, for CLX112. The CLX112 composition comprises, approximately, the ¹H-¹³C HSQC NMR correlations set forth in Table A for CLX112. The CLX112 composition comprises, approximately, the values set forth in Table C, as measured by oligosaccharide analysis. The molecular weight distribution of CLX 112 composition comprises, approximately, the values set forth in Table H, as measured by refractive index detection (RID) (see also FIG. 10C). CLX 112 has a dynamic viscosity at 25° C. at 100 mg/ml of about 1.248 mPa s. CLX112 generally is derived from barley beta glucan, but can also be derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and ¹H-¹³C HSQC NMR analysis as CLX112. In an embodiment, CLX112 is produced in accordance with the depolymerization described in Example 8 from barley beta glucan.

As used herein, the term “CLX115Cu” refers to an oligosaccharide composition wherein about 87.5% of the mass comprises glucose and about 4.5% of the mass comprises arabinose, as measured by hydrolytic monosaccharide compositional analysis. For composition CLX115Cu, the glycosidic linkage composition comprises, approximately, the amount set forth in Table B, for CLX115Cu. The CLX115Cu oligosaccharide composition comprises, approximately, the values set forth in Table E, as measured by oligosaccharide analysis. The molecular weight distribution of CLX115Cu composition comprises, approximately, the values set forth in Table H, as measured by refractive index detection (RID) (see also FIG. 10B). CLX115Cu generally is derived from oat beta glucan, but can also be derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, and oligosaccharide analysis as CLX115Cu. In an embodiment, CLX115Cu is produced from oat beta glucan in accordance with the depolymerization described in Example 7.

As used herein, the term “CLX122” refers to an oligosaccharide composition wherein about 80% of the mass comprises arabinose, about 10% of the mass comprises galactose, about 4% of the mass comprises glucose, about 4% of the mass comprises galacturonic acid, and about 2% of the mass comprises rhamnose, as measured by hydrolytic monosaccharide compositional analysis. For composition CLX122, the glycosidic linkage composition comprises, approximately, the amounts set forth in Table B for CLX122. The CLX122 composition comprises, approximately, the ¹H-¹³C HSQC NMR correlations set forth in Table A for CLX122. The CLX122 oligosaccharide composition comprises, approximately, the values set forth in Table F, as measured by oligosaccharide analysis. CLX122 has a dynamic viscosity at 25° C. at 100 mg/ml of about 2.913 mPa s. CLX122 generally is derived from pea arabinan, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-¹³C HSQC NMR analysis as CLX122. In an embodiment, CLX122 is produced in accordance with the depolymerization described in Example 8 from pea arabinan.

As used herein, the term “CLX122DSF” refers to an oligosaccharide composition wherein about 77% of the mass comprises arabinose, about 10% of the mass comprises glucose, about 6% of the mass comprises galactose, about 3% of the mass comprises galacturonic acid, and about 3% of the mass comprises rhamnose, as measured by hydrolytic monosaccharide compositional analysis. For composition CLX122DSF, the glycosidic linkage composition comprises, approximately, the amounts set forth in Table B. The CLX122DSF composition comprises, approximately, the 1H-¹³C HSQC NMR correlations set forth in Table A for CLX122DSF. The CLX122DSF oligosaccharide composition comprises, approximately, the values set forth in Table G, as measured by oligosaccharide analysis. CLX122DSF has a dynamic viscosity at 25° C. at 100 mg/ml of about 1.555 mPa s. The molecular weight distribution of CLX122DSF composition comprises, approximately, the values set forth in Table H, as measured by refractive index detection (RID) (see also FIG. 10D). CLX122DSF generally is derived from pea arabinan, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, ¹H-¹³C HSQC NMR analysis and glycosidic linkage composition as CLX122DSF. In an embodiment, CLX122DSF is produced in accordance with the depolymerization described in Example 8 with the exception that the pea arabinan was treated with alpha-amylase prior to depolymerization. (See: paragraph [0145] and Example 5 WO 2023/220318, published Nov. 16, 2023).

TABLE A
¹H-¹³C HSQC NMR correlations from oligosaccharide compositions used herein.
The listed pairs correspond to those major peaks in the anomeric region.

| CLX112 | | CLX115 | | CLX122 | | CLX122DSF | |
|---|---|---|---|---|---|---|---|
| ¹H δ | ¹³C δ | ¹H δ | ¹³C δ | ¹H δ | ¹³C δ | ¹H δ | ¹³C δ |
| [ppm] | [ppm] | [ppm] | [ppm] | [ppm] | [ppm] | [ppm] | [ppm] |
| 4.64 | 95.88 | 4.96 | 100.76 | 5.00 | 107.17 | 5.17 | 100.60 |
| 4.75 | 102.73 | 4.93 | 91.79 | 4.93 | 107.83 | 5.08 | 95.20 |
| 4.67 | 95.70 | 4.86 | 92.07 | 4.90 | 105.88 | 5.07 | 100.12 |
| 4.76 | 102.68 | 4.38 | 103.66 | 4.90 | 106.90 | 5.02 | 108.48 |
| 4.78 | 102.50 | 4.33 | 102.65 | 4.86 | 107.67 | 5.02 | 100.21 |
| 4.79 | 102.50 | 4.32 | 96.44 | 4.83 | 107.22 | 5.01 | 92.27 |
| 5.23 | 91.89 | 4.28 | 102.79 | 4.81 | 108.63 | 4.99 | 100.39 |
| 4.65 | 95.88 | 4.28 | 96.71 | 4.77 | 107.44 | 4.99 | 95.51 |
| 4.52 | 102.50 | 4.27 | 104.10 | 4.77 | 108.55 | 4.99 | 92.13 |
| 4.51 | 102.50 | 4.22 | 96.94 | 4.26 | 105.83 | 4.97 | 106.14 |
| 4.54 | 102.32 | 4.20 | 103.24 | 4.04 | 85.79 | 4.96 | 101.79 |
| 4.66 | 95.70 | | | 3.92 | 87.99 | 4.92 | 107.05 |
| | | | | 4.63 | 101.54 | 4.90 | 101.62 |
| | | | | 4.38 | 103.77 | 4.90 | 98.46 |
| | | | | 4.34 | 103.32 | 4.90 | 92.05 |
| | | | | 4.31 | 97.82 | 4.89 | 106.52 |
| | | | | 4.31 | 103.77 | 4.87 | 101.19 |
| | | | | 4.27 | 98.04 | 4.85 | 107.00 |
| | | | | 4.25 | 103.47 | 4.85 | 92.48 |
| | | | | 3.84 | 64.91 | 4.82 | 106.57 |
| | | | | 3.66 | 64.86 | 4.80 | 93.82 |
| | | | | | | 4.76 | 107.75 |
| | | | | | | 4.72 | 94.18 |
| | | | | | | 4.63 | 98.84 |
| | | | | | | 4.32 | 102.54 |
| | | | | | | 4.31 | 96.51 |
| | | | | | | 4.26 | 102.05 |
| | | | | | | 4.26 | 96.68 |
| | | | | | | 4.24 | 105.55 |
| | | | | | | 4.23 | 102.75 |
| | | | | | | 4.21 | 97.16 |

In Table B data are presented in units of peak area %. “Other” refers to linkages making up less than 2%. The notation “--” represents a linkage that exists in an amount less than 2% (which can be 0%) of the total oligosaccharide weight. If linkage is not fully described it will be denoted by the monosaccharide, when known, or the type of monosaccharide, either pentose or hexose, followed by multiple or a single “x” denoting the number of branch points and finally the retention time, in parentheses, in the units of minutes.

In each of the tables below, and elsewhere herein where these terms are used, Hex refers to hexose sugars, Pent refers to pentose sugars, HexA refers to hexuronic acid sugars, and Deoxyhex refers to deoxyhexose sugars. The number preceding such designation refers to the number of those units present in the relevant oligosaccharide (e.g., “3Hex” means the oligosaccharide is an oligomer of three hexoses). NR refers to an oligosaccharide without a reducing end. RT refers to retention time. Oligo wt. % is short for oligosaccharide weight % as that term is used in the definition of “oligosaccharide analysis” elsewhere herein.

TABLE B
Glycosidic linkage analysis of CLX compositions.

| | 3- | 4- | 6- | 4,6- | | | | | | 2,3,4- |
|---|---|---|---|---|---|---|---|---|---|---|
| CLX No. | Glc | Glc | Glc | Glc | T-Glc | 4-Gal | T-Gal-p | T-Xyl | 4-Xyl | Xyl |
| CLX115 | 23 | 64 | — | — | 13 | — | — | — | — | — |
| CLX115Cu | — | 64.41 | — | — | 16.42 | 9.27 | — | — | — | — |
| CLX112 | 17 | 49 | — | — | 31 | — | — | — | — | — |
| CLX122 | — | 3.4 | — | — | — | 4.54 | 2.89 | — | — | — |
| CLX122DSF | — | — | — | — | — | 2.1 | — | 2 | — | 10.04 |
| | | | | | T- | | x-Hex | 2-Pent | x-Pent | |
| | 5- | 3.5- | 2,5- | 2,3,5- | Arab- | 2- | (2.108 | (2.769 | (5.289 | |
| CLX No. | Arab | Arab | Arab | Arab | f | Rham | min) | min) | min) | Other |
| CLX115 | — | — | — | — | — | — | — | — | — | — |
| CLX115Cu | — | — | — | — | — | — | — | — | — | 5.96 |
| CLX112 | — | — | — | — | — | — | — | — | — | — |
| CLX122 | 23.16 | 4.2 | 9.25 | 4.38 | 34.99 | — | — | — | — | 0.45 |
| CLX122DSF | 17 | 3.84 | 1.75 | — | 50.18 | — | — | — | — | 9.01 |

TABLE C
CLX112 comprises the oligosaccharides shown in this table. Hex refers
to hexose sugars, Pent refers to pentose sugars, HexA refers to
hexuronic acid sugars, and Deoxyhex refers to deoxyhexose sugars.

| | | | RT | | Retention |
|---|---|---|---|---|---|
| Compound | Identity | Mass | (min) | Oligo Wt % | Factor |
| 1 | 3Hex | 504.19 | 10.431 | 7.9 | 1 |
| 2 | 3Hex | 504.19 | 14.158 | 13.53 | 1.357 |
| 3 | 3Hex | 504.19 | 17.074 | 5.47 | 1.637 |
| 4 | 4Hex | 666.24 | 21.248 | 11.27 | 2.037 |
| 5 | 4Hex | 666.24 | 24.78 | 6.29 | 2.376 |
| 6 | 4Hex | 666.24 | 25.028 | 8.82 | 2.399 |
| 7 | 4Hex | 666.24 | 25.857 | 9.81 | 2.479 |
| 8 | 5Hex | 828.29 | 27.544 | 2.08 | 2.641 |
| 9 | 5Hex | 828.29 | 28.172 | 3.8 | 2.701 |
| 10 | 5Hex | 828.29 | 28.914 | 1.18 | 2.772 |
| 11 | 5Hex | 828.29 | 29.507 | 7.33 | 2.829 |
| 12 | 5Hex | 828.29 | 30.241 | 11.24 | 2.899 |
| 13 | 6Hex | 990.34 | 32.369 | 2.62 | 3.103 |
| 14 | 6Hex | 990.34 | 34.032 | 6.74 | 3.263 |
| 15 | 6Hex | 990.34 | 37.05 | 1.95 | 3.552 |

TABLE D
CLX115 comprises the oligosaccharides shown in this table. Hex refers
to hexose sugars, Pent refers to pentose sugars, HexA refers to
hexuronic acid sugars, and Deoxyhex refers to deoxyhexose sugars.

| | | | RT | Oligos | Retention |
|---|---|---|---|---|---|
| Compound | Identity | Mass | (min) | Wt % | Factor |
| 1 | 1Hex1Pent | 314.1214 | 1.839 | 0.09 | 1.196 |
| 2 | 1Hex2Pent | 446.1638 | 14.618 | 0.46 | 9.511 |
| 3 | 1Hex2Pent | 446.1641 | 14.73 | 0.17 | 9.584 |
| 4 | 1Hex2Pent | 446.1635 | 15.648 | 0.28 | 10.181 |
| 5 | 1Hex2Pent | 446.1643 | 17.222 | 0.11 | 11.205 |
| 6 | 2Hex | 344.1321 | 1.537 | 1.05 | 1 |
| 7 | 2Hex | 344.1329 | 2.878 | 2.41 | 1.872 |
| 8 | 2Hex1Pent | 476.1744 | 12.931 | 1.06 | 8.413 |
| 9 | 2Hex1Pent | 476.174 | 13.229 | 0.49 | 8.607 |
| 10 | 2Hex1Pent | 476.1753 | 13.732 | 1.53 | 8.934 |
| 11 | 2Hex1Pent | 476.1742 | 15.112 | 0.33 | 9.832 |
| 12 | 2Hex1Pent | 476.1748 | 16.113 | 0.84 | 10.483 |
| 13 | 2Hex1Pent | 476.1742 | 16.527 | 0.33 | 10.753 |
| 14 | 2Hex2Pent | 608.2157 | 24.944 | 0.45 | 16.229 |
| 15 | 2Hex2Pent | 608.2161 | 26.779 | 0.19 | 17.423 |
| 16 | 3Hex | 506.1856 | 3.295 | 0.63 | 2.144 |
| 17 | 3Hex | 506.1856 | 4.549 | 0.8 | 2.96 |
| 18 | 3Hex | 506.1843 | 10.5 | 0.08 | 6.831 |
| 19 | 3Hex | 506.1855 | 11.335 | 7.55 | 7.375 |
| 20 | 3Hex | 506,1855 | 14.01 | 0.23 | 9.115 |
| 21 | 3Hex | 506.1857 | 15.193 | 9.74 | 9.885 |
| 22 | 3Hex | 506.1857 | 15.445 | 0.05 | 10.049 |
| 23 | 3Hex | 506.184 | 15.874 | 0.08 | 10.328 |
| 24 | 3Hex | 506.1856 | 17.831 | 6.41 | 11.601 |
| 25 | 3Hex1HexA | 682.2171 | 25.124 | 0.13 | 16.346 |
| 26 | 3Hex1HexA | 682.2176 | 27.182 | 0.58 | 17.685 |
| 27 | 3Hex1HexA | 682.2177 | 27.272 | 0.19 | 17.744 |
| 28 | 3Hex1HexA | 682.2169 | 28.058 | 0.67 | 18.255 |
| 29 | 3Hex1Pent | 638.2261 | 9.335 | 0.1 | 6.074 |
| 30 | 3Hex1Pent | 638.2263 | 10.131 | 0.07 | 6.591 |
| 31 | 3Hex1Pent | 638.2276 | 23.161 | 0.93 | 15.069 |
| 32 | 3Hex1Pent | 638.2275 | 23.637 | 0.4 | 15.379 |
| 33 | 3Hex1Pent | 638.2273 | 24.298 | 0.44 | 15.809 |
| 34 | 3Hex1Pent | 638.2271 | 25.161 | 0.72 | 16.37 |
| 35 | 3Hex1Pent | 638.2282 | 25.768 | 0.84 | 16.765 |
| 36 | 3Hex1Pent | 638.2271 | 26.311 | 0.23 | 17.118 |
| 37 | 3Hex2Pent | 770.2696 | 29.593 | 0.06 | 19.254 |
| 38 | 3Hex2Pent | 770.2686 | 30.152 | 0.19 | 19.617 |
| 39 | 3Hex2Pent | 770.2681 | 30.838 | 0.07 | 20.064 |
| 40 | 3Hex2Pent | 770.2695 | 30.903 | 0.12 | 20.106 |
| 41 | 4Hex | 668.237 | 7.62 | 0.19 | 4.958 |
| 42 | 4Hex | 668.238 | 8.72 | 0.1 | 5.673 |
| 43 | 4Hex | 668.237 | 9.276 | 1.35 | 6.035 |
| 44 | 4Hex | 668.2351 | 9.819 | 0.06 | 6.388 |
| 45 | 4Hex | 668.2378 | 10.269 | 1.06 | 6.681 |
| 46 | 4Hex | 668.2379 | 10.63 | 0.34 | 6.916 |
| 47 | 4Hex | 668.2388 | 21.557 | 8.22 | 14.025 |
| 48 | 4Hex | 668.2386 | 24.211 | 0.1 | 15.752 |
| 49 | 4Hex | 668.239 | 25.244 | 4.57 | 16.424 |
| 50 | 4Hex | 668.239 | 25.507 | 5.92 | 16.595 |
| 51 | 4Hex | 668.2387 | 26.403 | 6.18 | 17.178 |
| 52 | 4Hex | 668.2343 | 26.607 | 0.04 | 17.311 |
| 53 | 4Hex1Deoxyhex | 814.2585 | 31.081 | 0.31 | 20.222 |
| 54 | 4Hex1HexA | 844.247 | 29.807 | 0.04 | 19.393 |
| 55 | 4Hex1Pent | 800.2787 | 28.827 | 0.23 | 18.755 |
| 56 | 4Hex1Pent | 800.2797 | 29.062 | 0.41 | 18.908 |
| 57 | 4Hex1Pent | 800.2787 | 29.552 | 0.11 | 19.227 |
| 58 | 4Hex1Pent | 800.2799 | 29.615 | 0.56 | 19.268 |
| 59 | 4Hex1Pent | 800.2788 | 30.227 | 1.2 | 19.666 |
| 60 | 5Hex | 830.2911 | 11.751 | 0.2 | 7.645 |
| 61 | 5Hex | 830.2895 | 13.261 | 0.48 | 8.628 |
| 62 | 5Hex | 830.2893 | 13.518 | 0.46 | 8.795 |
| 63 | 5Hex | 830.2899 | 13.84 | 0.71 | 9.005 |
| 64 | 5Hex | 830.2907 | 27.932 | 2.29 | 18.173 |
| 65 | 5Hex | 830.2922 | 28.423 | 3.57 | 18.493 |
| 66 | 5Hex | 830.2907 | 29.322 | 1.02 | 19.077 |
| 67 | 5Hex | 830.2908 | 29.82 | 4.98 | 19.401 |
| 68 | 5Hex | 830.291 | 30.469 | 6.13 | 19.824 |
| 69 | 6Hex | 992.3433 | 14.683 | 0.1 | 9.553 |
| 70 | 6Hex | 992.342 | 15.905 | 0.98 | 10.348 |
| 71 | 6Hex | 992.3433 | 31.896 | 1.52 | 20.752 |
| 72 | 6Hex | 992.3427 | 31.937 | 1.02 | 20.779 |
| 73 | 6Hex | 992.3434 | 33.337 | 0.52 | 21.69 |
| 74 | 6Hex | 992.3429 | 33.37 | 1.9 | 21.711 |
| 75 | 6Hex | 992.3429 | 33.763 | 0.91 | 21.967 |
| 76 | 6Hex | 992.3421 | 35.487 | 1.12 | 23.088 |

TABLE E
CLX115Cu comprises the oligosaccharides shown in this
table. Hex refers to hexose sugars, Pent refers to
pentose sugars, HexA refers to hexuronic acid sugars,
and Deoxyhex refers to deoxyhexose sugars.

| | | | RT | Oligo | Retention |
|---|---|---|---|---|---|
| Compound | Identity | Mass | (min) | Wt. % | Factor |
| 1 | 1Hex1Pent | 312.12 | 2.063 | 3.5 | 1.158 |
| 2 | 1Hex1Pent | 312.12 | 11.288 | 0.31 | 6.334 |
| 3 | 1Hex1Pent | 312.12 | 12.054 | 0.44 | 6.764 |
| 4 | 1Hex1Pent | 312.12 | 14.528 | 0.4 | 8.153 |
| 5 | 1Hex2Pent | 444.17 | 13.12 | 0.95 | 7.363 |
| 6 | 1Hex2Pent | 444.17 | 14.019 | 0.9 | 7.867 |
| 7 | 1Hex2Pent | 444.17 | 15.704 | 0.28 | 8.813 |
| 8 | 2Hex | 342.13 | 2.189 | 0.2 | 1.228 |
| 9 | 2Hex | 342.13 | 2.916 | 0.82 | 1.636 |
| 10 | 2Hex | 342.13 | 3.01 | 2.45 | 1.689 |
| 11 | 2Hex | 342.13 | 9.733 | 1.32 | 5.462 |
| 12 | 2Hex | 342.13 | 13.479 | 1.89 | 7.564 |
| 13 | 2Hex1Pent | 474.18 | 10.122 | 0.05 | 5.68 |
| 14 | 2Hex1Pent | 474.17 | 11.287 | 1.96 | 6.334 |
| 15 | 2Hex1Pent | 474.17 | 11.594 | 0.43 | 6.506 |
| 16 | 2Hex1Pent | 474.17 | 12.054 | 2.65 | 6.764 |
| 17 | 2Hex1Pent | 474.18 | 14.528 | 1.12 | 8.153 |
| 18 | 2Hex1Pent | 474.18 | 14.944 | 0.44 | 8.386 |
| 19 | 2Hex2Pent | 606.22 | 23.54 | 0.32 | 13.21 |
| 20 | 2Hex2Pent | 606.22 | 24.287 | 0.38 | 13.629 |
| 21 | 2Hex2Pent | 606.22 | 25.041 | 0.61 | 14.052 |
| 22 | 3Hex | 504.19 | 1.782 | 0.09 | 1 |
| 23 | 3Hex | 504.19 | 3.333 | 0.43 | 1.87 |
| 24 | 3Hex | 504.18 | 4.242 | 0.15 | 2.38 |
| 25 | 3Hex | 504.18 | 5.108 | 0.12 | 2.866 |
| 26 | 3Hex | 504.18 | 6.804 | 0.31 | 3.818 |
| 27 | 3Hex | 504.18 | 8.412 | 0.76 | 4.721 |
| 28 | 3Hex | 504.18 | 8.669 | 0.39 | 4.865 |
| 29 | 3Hex | 504.19 | 9.734 | 9.22 | 5.462 |
| 30 | 3Hex | 504.19 | 12.287 | 1.16 | 6.895 |
| 31 | 3Hex | 504.19 | 23.908 | 0.43 | 13.416 |
| 32 | 3Hex | 504.19 | 24.73 | 0.32 | 13.878 |
| 33 | 3Hex | 504.19 | 27.971 | 0.46 | 15.696 |
| 34 | 3Hex1Deoxyhex | 650.24 | 1.903 | 0.06 | 1.068 |
| 35 | 3Hex1HexA | 680.22 | 23.207 | 1.06 | 13.023 |
| 36 | 3Hex1HexA | 680.22 | 25.112 | 0.94 | 14.092 |
| 37 | 3Hex1HexA | 680.22 | 25.972 | 1.07 | 14.575 |
| 38 | 3Hex1Pent | 636.23 | 19.245 | 0.03 | 10.8 |
| 39 | 3Hex1Pent | 636.23 | 21.862 | 1.58 | 12.268 |
| 40 | 3Hex1Pent | 636.23 | 22.377 | 0.32 | 12.557 |
| 41 | 3Hex1Pent | 636.23 | 22.939 | 0.71 | 12.873 |
| 42 | 3Hex1Pent | 636.23 | 23.624 | 0.76 | 13.257 |
| 43 | 3Hex1Pent | 636.23 | 24.175 | 1.49 | 13.566 |
| 44 | 3Hex1Pent | 636.22 | 24.268 | 0.1 | 13.618 |
| 45 | 3Hex1Pent | 636.23 | 24.438 | 0.31 | 13.714 |
| 46 | 3Hex1Pent | 636.23 | 24.656 | 0.09 | 13.836 |
| 47 | 3Hex2Pent | 768.27 | 28.4 | 0.2 | 15.937 |
| 48 | 3Hex2Pent | 768.27 | 29.119 | 0.16 | 16.341 |
| 49 | 4Hex | 666.24 | 7.554 | 0.18 | 4.239 |
| 50 | 4Hex | 666.24 | 8.467 | 0.12 | 4.751 |
| 51 | 4Hex | 666.24 | 17.617 | 0.24 | 9.886 |
| 52 | 4Hex | 666.24 | 17.98 | 0.14 | 10.09 |
| 53 | 4Hex | 666.24 | 18.202 | 0.71 | 10.214 |
| 54 | 4Hex | 666.24 | 18.74 | 0.21 | 10.516 |
| 55 | 4Hex | 666.24 | 19.121 | 0.3 | 10.73 |
| 56 | 4Hex | 666.24 | 20.19 | 9.67 | 11.33 |
| 57 | 4Hex | 666.24 | 21.297 | 0.4 | 11.951 |
| 58 | 4Hex | 666.24 | 22.289 | 0.21 | 12.508 |
| 59 | 4Hex | 666.24 | 23.248 | 0.13 | 13.046 |
| 60 | 4Hex | 666.24 | 23.697 | 4.91 | 13.298 |
| 61 | 4Hex | 666.24 | 23.84 | 2.45 | 13.378 |
| 62 | 4Hex | 666.24 | 23.869 | 0.25 | 13.395 |
| 63 | 4Hex | 666.24 | 24.452 | 0.28 | 13.722 |
| 64 | 4Hex | 666.24 | 24.73 | 7.14 | 13.878 |
| 65 | 4Hex1HexA | 842.27 | 30.693 | 1.13 | 17.224 |
| 66 | 4Hex1Pent | 798.28 | 27.188 | 0.57 | 15.257 |
| 67 | 4Hex1Pent | 798.28 | 27.761 | 0.48 | 15.579 |
| 68 | 4Hex1Pent | 798.28 | 28.4 | 1.13 | 15.937 |
| 69 | 5Hex | 828.29 | 22.477 | 0.05 | 12.613 |
| 70 | 5Hex | 828.29 | 22.735 | 0.05 | 12.758 |
| 71 | 5Hex | 828.29 | 23.539 | 0.1 | 13.209 |
| 72 | 5Hex | 828.29 | 24.626 | 0.09 | 13.819 |
| 73 | 5Hex | 828.29 | 24.857 | 0.29 | 13.949 |
| 74 | 5Hex | 828.29 | 26.032 | 2.76 | 14.608 |
| 75 | 5Hex | 828.29 | 26.525 | 0.07 | 14.885 |
| 76 | 5Hex | 828.29 | 26.537 | 3.33 | 14.892 |
| 77 | 5Hex | 828.29 | 27.442 | 1.4 | 15.4 |
| 78 | 5Hex | 828.29 | 27.974 | 4.82 | 15.698 |
| 79 | 5Hex | 828.29 | 28.668 | 0.12 | 16.088 |
| 80 | 5Hex | 828.29 | 28.674 | 7.11 | 16.091 |
| 81 | 5Hex | 828.29 | 28.697 | 0.04 | 16.104 |
| 82 | 5Hex | 828.29 | 41.967 | 0.32 | 23.551 |
| 83 | 5Hex1Deoxyhex | 974.35 | 11.313 | 0.12 | 6.348 |
| 84 | 5Hex 1Deoxyhex | 974.35 | 12.165 | 0.08 | 6.827 |
| 85 | 5Hex1Deoxyhex | 974.35 | 13.385 | 0.03 | 7.511 |
| 86 | 5Hex1Pent | 960.34 | 30.951 | 0.07 | 17.369 |
| 87 | 5Hex1Pent | 960.33 | 31.583 | 0.15 | 17.723 |
| 88 | 6Hex | 990.34 | 26.403 | 0.06 | 14.816 |
| 89 | 6Hex | 990.34 | 27.633 | 0.05 | 15.507 |
| 90 | 6Hex | 990.34 | 30.321 | 0.51 | 17.015 |
| 91 | 6Hex | 990.34 | 31.531 | 1.44 | 17.694 |
| 92 | 6Hex | 990.34 | 31.983 | 1.21 | 17.948 |
| 93 | 6Hex | 990.34 | 33.875 | 0.94 | 19.01 |

TABLE F
CLX122 comprises the oligosaccharides shown in this
table (Hex = hexose, generally galactose or mannose).

| | | | RT | Oligo | Retention |
|---|---|---|---|---|---|
| Compound | Identity | Mass | (min) | Wt. % | Factor |
| 1 | 2Hex1Pent | 474.17 | 3.789 | 3.20 | 1.157 |
| 2 | 2Hex1Pent | 474.17 | 5.019 | 1.67 | 1.532 |
| 3 | 3Hex | 504.18 | 3.276 | 3.64 | 1 |
| 4 | 3Hex | 504.18 | 5.023 | 5.46 | 1.533 |
| 5 | 3Hex | 504.18 | 15.334 | 2.97 | 4.681 |
| 6 | 3Hex (NR) | 504.17 | 3.911 | 1.52 | 1.194 |
| 7 | 3Hex (NR) | 504.17 | 5.613 | 3.36 | 1.713 |
| 8 | 3Hex (NR) | 504.17 | 10.914 | 3.05 | 3.332 |
| 9 | 3Hex (NR) | 504.17 | 12.221 | 8.03 | 3.73 |
| 10 | 3Hex1Pent | 636.23 | 8.958 | 3.67 | 2.734 |
| 11 | 3Hex1Pent | 636.23 | 10.904 | 1.65 | 3.328 |
| 12 | 3Pent | 414.15 | 3.867 | 0.98 | 1.18 |
| 13 | 3Pent | 414.15 | 6.269 | 9.92 | 1.914 |
| 14 | 3Pent | 414.16 | 7.057 | 0.81 | 2.154 |
| 15 | 4Hex | 666,24 | 8.187 | 3.64 | 2.499 |
| 16 | 4Hex | 666.24 | 10.829 | 5.60 | 3.306 |
| 17 | 4Hex | 666.24 | 19.604 | 0.58 | 5.984 |
| 18 | 4Hex | 666.24 | 25.305 | 2.23 | 7.724 |
| 19 | 4Hex (NR) | 666.22 | 9.171 | 3.30 | 2.799 |
| 20 | 4Hex (NR) | 666.22 | 12.226 | 0.95 | 3.732 |
| 21 | 4Hex (NR) | 666.22 | 12.227 | 4.67 | 3.732 |
| 22 | 4Hex (NR) | 666.22 | 15.283 | 3.31 | 4.665 |
| 23 | 4Hex1Pent | 798.28 | 11.988 | 3.05 | 3.659 |
| 24 | 4Hex1Pent | 798.28 | 14.07 | 1.23 | 4.295 |
| 25 | 4Pent | 546.19 | 10.644 | 1.79 | 3.249 |
| 26 | 4Pent | 546.2 | 11.358 | 3.33 | 3.467 |
| 27 | 4Pent | 546.2 | 13.022 | 0.89 | 3.975 |
| 28 | 5Hex | 828.29 | 11.467 | 2.50 | 3.5 |
| 29 | 5Hex | 828.29 | 13.946 | 4.75 | 4.257 |
| 30 | 5Hex | 828.29 | 29.244 | 1.39 | 8.927 |
| 31 | 5Pent | 678.24 | 15.834 | 2.26 | 4.833 |
| 32 | 5Pent | 678.23 | 17.759 | 1.00 | 5.421 |
| 33 | 6Hex | 990.34 | 15.785 | 2.77 | 4.818 |
| 34 | 6Hex1Pent | 1122.39 | 15.078 | 0.83 | 4.603 |

TABLE G
CLX122DSF comprises the oligosaccharides shown in
this table (Hex = hexose, generally galactose
or glucose and Pent-Pentose generally Arabinose).

| | | | RT | Oligo | Retention |
|---|---|---|---|---|---|
| Compound | Identity | Mass | (min) | Wt. % | Factor |
| 1 | 2Hex1Pent | 474.18 | 3.738 | 1.46 | 1.133 |
| 2 | 2Hex1Pent | 474.18 | 4.894 | 0.53 | 1.483 |
| 3 | 2Hex1Pent | 474.17 | 7.865 | 0.43 | 2.383 |
| 4 | 2Hex1Pent | 474.17 | 8.657 | 0.82 | 2.623 |
| 5 | 2Hex1Pent | 474.18 | 11.39 | 1 | 3.452 |
| 6 | 2Hex1Pent | 474.17 | 14.152 | 0.38 | 4.288 |
| 7 | 3Hex | 504.18 | 3.3 | 2.62 | 1 |
| 8 | 3Hex | 504.19 | 3.711 | 0.48 | 1.125 |
| 9 | 3Hex | 504.19 | 4.89 | 1.58 | 1.482 |
| 10 | 3Hex | 504.19 | 7.959 | 0.83 | 2.412 |
| 11 | 3Hex | 504.18 | 15.53 | 2.25 | 4.706 |
| 12 | 3Hex | 504.19 | 20.938 | 0.64 | 6.345 |
| 13 | 3Hex | 504.19 | 22.266 | 1.23 | 6.747 |
| 14 | 3Hex | 504.19 | 25.228 | 0.62 | 7.645 |
| 15 | 3Hex | 504.19 | 26.138 | 0.82 | 7.921 |
| 16 | 3Pent | 414.16 | 4.045 | 5.2 | 1.226 |
| 17 | 3Pent | 414.15 | 5.625 | 3.51 | 1.705 |
| 18 | 3Pent | 414.15 | 6.668 | 10.74 | 2.021 |
| 19 | 3Pent | 414.15 | 7.368 | 2.49 | 2.233 |
| 20 | 3Pent | 414.15 | 10.065 | 0.49 | 3.05 |
| 21 | 3Pent | 414.15 | 10.977 | 4.44 | 3.326 |
| 22 | 3Pent | 414.15 | 11.713 | 4.51 | 3.549 |
| 23 | 3Pent | 414.15 | 12.482 | 2.1 | 3.782 |
| 24 | 3Pent | 414.15 | 12.785 | 1.97 | 3.874 |
| 25 | 3Pent | 414.15 | 13.3 | 2.24 | 4.03 |
| 26 | 3Pent | 414.15 | 14.031 | 1.95 | 4.252 |
| 27 | 3Pent | 414.15 | 16.631 | 1.15 | 5.04 |
| 28 | 4Hex | 666.24 | 7.958 | 1.86 | 2.412 |
| 29 | 4Hex | 666.24 | 10.667 | 1.69 | 3.232 |
| 30 | 4Hex | 666.24 | 25.519 | 1.97 | 7.733 |
| 31 | 4Hex1Pent | 798.28 | 11.491 | 0.58 | 3.482 |
| 32 | 4Pent | 546.2 | 4.044 | 0.67 | 1.225 |
| 33 | 4Pent | 546.2 | 6.632 | 1.38 | 2.01 |
| 34 | 4Pent | 546.2 | 10.927 | 0.55 | 3.311 |
| 35 | 4Pent | 546.2 | 10.982 | 3.69 | 3.328 |
| 36 | 4Pent | 546.2 | 11.359 | 1.02 | 3.442 |
| 37 | 4Pent | 546.2 | 11.712 | 3.62 | 3.549 |
| 38 | 4Pent | 546.2 | 12.493 | 2.06 | 3.786 |
| 39 | 4Pent | 546.2 | 12.778 | 1.86 | 3.872 |
| 40 | 4Pent | 546.2 | 13.301 | 2.3 | 4.031 |
| 41 | 4Pent | 546.2 | 14.03 | 2.07 | 4.252 |
| 42 | 4Pent | 546.2 | 16.028 | 3.61 | 4.857 |
| 43 | 4Pent | 546.2 | 16.64 | 1.37 | 5.042 |
| 44 | 5Hex | 828.29 | 11.06 | 0.88 | 3.352 |
| 45 | 5Pent | 678.24 | 15.339 | 0.92 | 4.648 |
| 46 | 5Pent | 678.24 | 15.787 | 1.36 | 4.784 |
| 47 | 5Pent | 678.24 | 16.082 | 2.71 | 4.873 |
| 48 | 5Pent | 678.24 | 16.642 | 1.43 | 5.043 |
| 49 | 5Pent | 678.24 | 17.207 | 0.69 | 5.214 |
| 50 | 5Pent | 678.24 | 17.957 | 1.83 | 5.442 |
| 51 | 5Pent | 678.24 | 20.845 | 0.48 | 6.317 |
| 52 | 6Hex | 990.34 | 15.36 | 0.57 | 4.655 |
| 53 | 6Pent | 810.28 | 17.546 | 0.42 | 5.317 |
| 54 | 6Pent | 810.28 | 19.118 | 0.93 | 5.793 |
| 55 | 6Pent | 810.28 | 19.47 | 0.99 | 5.9 |

TABLE H
Refractive index detection (RID) analysis of CLX compositions.
Data are presented in units of peak area %.

| M.W. | | | | |
|---|---|---|---|---|
| Distribution | CLX122DSF | CLX115Cu | CLX115 | CLX112 |
| >100k | 8.31 | 21.19 | 21.12 | 11.65 |
| 100k-50k | 11.72 | 6.56 | 13.91 | 5.79 |
| 50k-15k | 32.36 | 0.74 | 3.18 | 1.68 |
| 15k-5k | 15.89 | 1.81 | 0.8 | 0.32 |
| 5k-1k | 3.47 | 33.71 | 23.21 | 28.34 |
| 1k-500 | 7.35 | 30.16 | 34.65 | 47.28 |
| <500 | 20.9 | 5.84 | 3.12 | 4.93 |

“Comprising” means “including,” “containing,” or “characterized by,” and is inclusive or open-ended and does not exclude additional, unrecited elements or method steps. Related terms such as “comprise” and “comprised” are to be interpreted in the same manner. As used herein, “consisting of” excludes any element, step, or ingredient not specified in the 5 claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

“Degree of polymerization” or “DP” of an oligosaccharide refers to the total number of monosaccharide units that are part of a particular carbohydrate polymer. For example, a tetra oligosaccharide has a DP of 4.

“Effective amount” means an amount sufficient to render a desired treatment or management outcome in a subject, and in particular refers to an outcome in a human subject. When two or more active ingredients are combined for a given application, a combined effective amount is employed. An effective amount can be administered in one or more doses to achieve the desired treatment or management outcome.

“Elderly adult” means a human of age over 60 years, for example over 65 years.

“Enteral administration” means any conventional form for delivery of a composition to a human that causes the deposition of the composition in the gastrointestinal tract (including the stomach). Methods of enteral administration include feeding through a naso-gastric tube or jejunum tube, oral, sublingual and rectal.

The term “Loss of executive function” is used herein as it is understood in the art to refer to a disorder characterized by behavioral symptoms that disrupt a subject's ability to manage their own thought, emotions and actions, and which is also called “executive dysfunction.” Executive functions include working memory, cognitive flexibility and inhibition control. Symptoms of loss of executive function include, among others, distractibility, trouble focusing, inability to pay attention, trouble planning or carrying out tasks, and impulse control problems.

“Free monosaccharide compositional analysis” refers to the method described in Amicucci, Galermo et al. 2019, the disclosure of which is incorporated by reference, with some modifications. The derivatization reaction to produce monosaccharides is performed at the optimized condition of 70° C. for 30 minutes. Samples are run on an Agilent 1290 Infinity II ultra-high performance liquid chromatography (UHPLC) system coupled to an Agilent 6490A triple quadrupole (QqQ) mass spectrometer. Separation is carried out on an Agilent InfinityLab Poroshell HPH-C18 column (2.1 mm×50 mm, 1.9 μm particle size) plus a guard column (5 mm) with the same solvent system described in the paper. With a constant flow rate of 1.2 mL/min, an isocratic gradient of 8.5% B is used for the first 4-minute elution period, followed by 15% B for 0.4 minutes. For the flush period, 97% B was held for 1 minute. The column thermostat is set at 35° C. For the mass spectrometry parameters, the only change from the method described in Amicucci, Galermo et al. 2019 is that the fragmentor voltage is set at 380V. For data analysis, the hydrolysis correction factor is not applied since the samples contain oligosaccharides instead of polysaccharides. In this analysis method, inherently free unpolymerized monosaccharides are calculated by quantifying the concentrations of 14 monosaccharides (glucose, galactose, fructose, xylose, arabinose, fucose, rhamnose, glucuronic acid, galacturonic acid, N-acetylglucosamine, N-acetylgalactosamine, mannose, allose, ribose) against their individual standard curves. For example, 30% free glucose, as measured by free monosaccharide compositional analysis, means containing 30 g of glucose per 100 g of the sum of all 14 monosaccharides described above.

Glycosidic linkage composition”, “glycosidic linkage analysis”, “permethylated linkage composition analysis” or similar terms, refer to a method described in Galermo, Nandita et al. 2018, incorporated by reference in its entirety for all purposes, with some modifications. The permethylation reaction time is 30 minutes. Samples are run on an Agilent 1290 Infinity II UHPLC system couple to an Agilent 6490A QqQ mass spectrometer. Separation is carried out on an Agilent InfinityLab Poroshell HPH-C18 column (2.1 mm×100 mm, 1.9 μm particle size) plus a guard column (5 mm) with the same solvent system described in Galermo, Nandita et al. 2018. With a constant flow rate of 0.8 mL/min, an isocratic gradient of 14% B is used for the 16-minute elution period, followed by a 2-minute 99% B flush period. The column thermostat is set at 35° C. The glycosidic linkage composition is calculated by integrating the chromatographic peak area of all peaks with the following m/z values: 481.2., 495.2, 509.2, 523.3, 525.2, 537.3, 539.3, 553.3, 567.3, 581.3. For example. 20% 4-galactose, as measured by the permethylated linkage composition analysis, refers to the peak area of 4-galactose being 20% of the sum of the peak area of all linkage peaks with the m/z values listed above.

“Hydrolytic monosaccharide compositional analysis” means the method described in Amicucci, Galermo et al. 2019, incorporated by reference in its entirety for all purposes, with the following modifications. The hydrolysis reaction to produce monosaccharides is performed at the optimized condition of 100° C. for 2 hours. Samples are run on an Agilent 1290 Infinity II ultra-high performance liquid chromatography (UHPLC) system coupled to an Agilent 6490A triple quadrupole (QqQ) mass spectrometer. Separation is carried out on an Agilent InfinityLab Poroshell HPH-C18 column (2.1 mm×50 mm, 1.9 μm particle size) plus a guard column (5 mm) with the same solvent system described in Amicucci, Galermo et al. 2019. With a constant flow rate of 1.2 mb/min, an isocratic gradient of 8.5% B is used for the first 4-minute elution period, followed by 15% B for 0.4 minutes. For the flush period, 97% B is held for 1 minute. The column thermostat is set at 35° C. For the mass spectrometry parameters, the only change from the method described in Amicucci, Galermo et al. 2019 is that the fragmentor voltage is set at 380V. For data analysis, the hydrolysis correction factor is not applied nor needed since the samples contain oligosaccharides instead of polysaccharides. In this analysis method, monosaccharide composition is calculated by quantifying the concentrations of 14 monosaccharides (glucose, galactose, fructose, xylose, arabinose, fucose, rhamnose, glucuronic acid, galacturonic acid, N-acetylglucosamine, N-acetylgalactosamine, mannose, allose, ribose) against their individual standard curves. For example, 30% glucose, as measured by hydrolytic monosaccharide compositional analysis, means containing 30 g of glucose per 100 g of the sum of all 14 monosaccharides described above.

“Intestinal barrier” means the functional unit, organized as a multi-layer system, which forms the barrier between the intestinal lumen and the lamina propria. The intestinal barrier is made up of two main components: a physical barrier surface, which prevents bacterial adhesion and regulates paracellular diffusion to the host tissues, and a deep functional barrier, that is able to discriminate between pathogens and commensal microorganisms, organizing the immune tolerance and the immune response to pathogens. From the outer layer to the inner layer, the intestinal barrier comprises mucus, epithelial cells and the innate and adaptive immune cells forming the gut-associated lymphoid tissue.

“Intestinal barrier function” means the functioning of the intestinal barrier.

“Legume” means a plant in the family Fabaceae (or Leguminosae), or the fruit or seed of such a plant. Examples of legumes include peas, beans, soy, chickpeas, peanuts, lentils, lupins, mesquite, carob, tamarind, alfalfa, and clover. Legume may refer to by-products of the plant during harvest or food processing. Non-limiting examples include powders, pods, flowers, stems, roots, seeds, fiber, or crude protein. Legume may refer to the solid material after roasting, fermentation, hot-water, enzymatic, chemical, alkaline, super critical fluid, sun drying, organic solvent, acidic, mechanical pressure or pressure-based extractions.

“Lewis base” means a compound or atom that can donate electron pairs (e.g., F⁻, benzene, H⁻, pyridine, acetonitrile, acetone, urea, etc.).

“Linkage ratio”, “linkage peak area ratio”, “ratio of linkage” or other similar terms refer to any number of comparisons dependent upon the relationships observed in the glycosidic linkage composition analysis. Peak area for each linkage is calculated on a relative percent basis of the peak area in relationship to the summation of all other linkage peaks areas observed. Peak area ratios are calculated by dividing one contributing linkage by any other linkage of the same monosaccharide within the composition.

“Maintain” means to cause or enable a particular situation or action to continue substantially as before.

“Molecular weight analysis” or “SEC-RID” or similar terms, refer to a method in which Samples are prepared by reconstituting dried powders into a 10 mg/mL solution in HPLC grade water. Samples are analyzed on an Agilent Infinity 111260 RID coupled to an Agilent Infinity 111260 HPLC. Separation is performed on an Agilent AdvanceBio SEC column. Chromatographic solvents consisted of A: HPLC grade water and B: 95% acetonitrile in water (v/v). The RID is operated in positive signal polarity mode and a 2.31 Hz peak width. Samples are integrated using the manual integration tool in ChemStation data analysis.

“Monosaccharide ratio”, “monosaccharide peak area ratio”, “ratio of monosaccharide” or similar terms refer to any number of the comparisons dependent upon the relationships observed in the hydrolytic monosaccharide compositional analysis. Absolute concentrations of each monosaccharide are calculated on a relative percent basis in relation to the summation of all other monosaccharides observed. Monosaccharide ratios are calculated by dividing one contributing monosaccharide by any other monosaccharide within the composition.

“Nitrogen-based” means a compound that contains at least one nitrogen atom with four substituent groups that can contain any combination of lone pairs of electrons, hydrogens, or carbon atoms (e.g., ammonia, sodium amide, trimethylamine, diethylamine, N,N-Diisopropylethylamine, urea, pyridine, ammonium hydroxide, ammonium bicarbonate, etc.). Example nitrogen-based, peroxide-quenching, polysaccharide-cleavage agents are listed in Table 1 of published PCT application WO2022241163 (published Nov. 17, 2022). WO2022241163 is incorporated by reference herein in its entirety to the extent not inconsistent with the descriptions herein for any purpose and particularly for detail in Table 1 therein and descriptions therein of methods for depolymerization of polysaccharides. A nitrogen-based reagent may have an unsubstituted or substituted ammonium group and can be present in neutral and/or ionic forms.

“NMR HSQC Analysis”, “1H-¹³C HSQC NMR”, “HSQC spectra” or other similar terms mean the data generated from two-dimensional spectral analysis of a sample via a Heteronuclear Single Quantum Coherence (HSQC) spin coupling of protons and bonded carbons present in the sample. HSQC experimentation depends on the solvation of samples in a deuterated solvent such as D6-DMSO or D₂O. An HSQC spectrum contains a unique peak for each proton attached to the heteronuclear carbon atom being considered, allowing for identification of molecular structure of the analyzed sample. Each experiment is conducted with a Bruker AVANCE 600 MHz NMR using heteronuclear single quantum coherence (HSQC) to illustrate the correlation between the ¹H and ¹³C chemical shifts through 1JCH coupling. The resulting FIDs are processed using Bruker TopSpin 4.1.3 and the experimental chemical shifts are utilized to determine oligosaccharide structures and the anomeric characteristics of the glycosidic bonds with the aid of the CASPER program. Relative ratios between alpha and beta bonds are calculated through examination of the 2D ¹H-¹³C HSQC via examination of signal strength in Hz. These values are then compared to determine percent abundance of each linkage type among the same carbohydrate. NMR samples are dried via lyophilization, and the resulting material is then dissolved in 0.75 mL of dimethyl sulfoxide-d6 (DMSO-d6) with a 0.03% (v/v) TMS internal standard at a concentration of 20 mg/mL at a 4.5-6 pH range.

“Non-Arrhenius base” means a compound or atom that can donate electrons (e.g., Lewis Bases), accept protons (e.g., Bronstead-Lowry Bases), or releases hydroxide ions through its decomposition (NH₄HCO₃), but does not qualify as an Arrhenius base.

“Nutritional formula” means a foodstuff which is intended to satisfy the particular nutritional needs of a subject, particularly a human subject. The nutritional formula may be a complete nutritional formula which satisfies all the nutritional needs of the subject or a supplement to diet. The nutritional formulas are often regulated as a food for special medical purposes/medical food, but this may vary from country to country. However, the nutritional formula can fall within other food types such as dietary supplements, and foods.

“Oligosaccharide” means an oligomer of monosaccharides, in which the DP of the oligomer is between 2 and 50 monosaccharide units, such as between 3-50, 3-30, 3-20, 3-15, 3-10, 3-8, 3-6, or 5-15 monosaccharide units. An oligosaccharide can be linear, branched, primarily linear with pendant saccharide monomers, or any combination thereof. An oligosaccharide is individual oligomer chain.

“Oligosaccharide composition”, “oligosaccharide pool” or oligosaccharide mixture” means a mixture of two or more oligosaccharides, each of which can be the same or different from one another. Although efforts have been made to consistently use the terms “oligosaccharide” and “oligosaccharide composition” according to their preceding definitions, the intended meaning will be clear from context when such terms are used.

“Oligosaccharide analysis” or “oligosaccharide composition analysis” (or similar terms) refer to a HPLC-quadrupole time-of-flight (Q-TOF) method described in Amicucci, Nandita et al. 2020, incorporated by reference in its entirety for all purposes, with some modifications. For sample preparation, oligosaccharides are reduced by incubation with 2.0 M NaBH4 for 1 hour at 65° C. The oligosaccharides are purified using C-18 cartridge 96-well plates: the plates are washed with 100% ACN, and the oligosaccharides are loaded and eluted with water. The oligosaccharides are subsequently purified using porous graphitized carbon (PGC) 96-well plates: PCG plates are washed with 80% acetonitrile and 0.1% (v/v) TFA in water, and the oligosaccharides from C-18 purification are loaded and washed with water. The oligosaccharides are eluted with 40% acetonitrile with 0.05% (v/v) TFA. Samples are completely dried by evaporative centrifugation and reconstituted for mass spectrometry analysis. Instrumentation is performed on an Agilent 1260 Infinity II HPLC coupled to an Agilent 6530 Q-TOF mass spectrometer. Using the same stationary (plus a 5 mm guard column) and mobile phase as described in Amicucci, Nandita et al. 2020, separation is carried out using the following gradient: 2-15% B, 0-20 minutes; 15-60% B; 20-45 minutes. The column thermostat is set at 35° C. The fragmentor voltage is set at 75V. In this method, the oligosaccharide weight % (or oligo wt. % or such terms) is calculated by dividing the chromatographic peak area of a particular oligosaccharide by the total peak area of all oligosaccharides identified in that sample during the defined chromatographic period. Generally, when an oligosaccharide composition is described to contain a specified weight percent of oligosaccharides on a dry basis having a degree of polymerization of a specified number (e.g., at least 50 wt. % oligosaccharides on a dry basis having a degree of polymerization of between 3 and 50 monosaccharide subunits), such values can be calculated with the aid of the oligosaccharide analysis described above; however, other methods can also aid this determination, such as size exclusion chromatography using a universal detector, or other methods known in the art.

“Oral administration” means any conventional form for the delivery of a composition to a human through the mouth. Accordingly, oral administration is a form of enteral administration.

“Other minor linkages” means the sum of linkages which are either not entirely annotated or constitute less than 2% of any samples. Therefore, the contributions of these linkages to the sample glycosidic linkage composition are summed into the “other minor linkages” category.

“Pea” means any part of the plant in the genus Pisum, Cajanus, lathyrus or Vigna. Examples include the species Pisum sativum, Cajanus cajanor, Vigna unguiculata, and Lathyrus aphaca. Pea also includes other non-Pisum, Cajanus, lathyrus or Vigna genus, which are colloquially known as Pea, Snow pea, split pea, snap pea, field pea or sugar pea. Pea may refer to by-products of the plant during harvest or food processing. Non-limiting examples include Pea Powder, Pea pods, Pea flower, Pea stem, Pea stipules, Pea root, Pea seeds, Pea fiber, or crude pea protein. Pea may refer to the solid material after roasting, fermentation, hot-water, enzymatic, chemical, alkaline, super critical fluid, sun drying, organic solvent, acidic, mechanical pressure or pressure-based extractions.

“Peroxide agent” means a compound that contains oxygen-oxygen bonds that can produce, natively, with light, temperature, or catalyst (e.g., metals and enzymes), R—O· and/or R—O—O· species (radical species), where “R” refers to a hydrogen or carbon group (e.g., alkyl, aryl or other organic group) that is attached to the rest of the molecule. In one aspect, a peroxide agent is hydrogen peroxide.

“Peroxide quenching reagent” means a compound or atom which is not a strong-Arrhenius base, and that can convert hydrogen peroxide, peroxyl radicals, and hydroperoxyl radicals to a less reactive or non-reactive state (e.g., ammonium hydroxide, ammonium bicarbonate, ammonia, etc.). In certain aspects, a peroxide quenching reagent converts hydrogen peroxide as well as radicals produced from hydrogen peroxide to less reactive species (e.g. water). In certain aspects, a peroxide quenching reagent may reduce the hydrogen peroxide concentration to zero, below 5 mg/L, below 10 mg/L, below 25 mg/L, or below 50 mg/L. In certain aspects, a peroxide quenching reagent may form water, hydroxide ions, or oxygen gas. In certain aspects, the peroxide quenching reagent may be an enzyme, for example, a catalase. The enzyme can be from microbial origin, from recombinant origin, or from animal origin, for example from bovine liver. In certain aspects, different enzymes may be mixed to quench the peroxide species.

“Polysaccharide” means a polymer of monosaccharide units having greater than 30 monosaccharide units or a material comprising such a polymer. The polysaccharide can be linked to other non-carbohydrate moieties (e.g., glycoproteins, proteoglycans, glycopeptides, glycolipids, glycoconjugates, glycosides, or any combination thereof). The polysaccharide can be a linear polymer, branched polymer, primarily linear polymer with pendant saccharide monomers, or any combination thereof.

“Polysaccharide cleavage product” is a product formed from the chemical and/or enzymatic cleavage of a polysaccharide.

“Prevention” means treatment given or action taken to diminish the risk of onset or recurrence of a condition, including a disease.

“Primary prevention” means prevention of the initial onset of a condition in an individual.

“Probiotic” means alive microorganism which when administered in adequate amounts confers a health benefit on the host.

“Reaction mixture” means a mixture comprising reagents which may react chemically to form products which are distinct from the reagents.

“Reducing” or any variation of the term such as “reduction” means any measurable decrease to achieve a desired effect.

“Retention factor” means the ratio obtained by dividing the retention time of a given peak observed in an oligosaccharide analysis (e.g., HPLC spectrum) by the first oligosaccharide peak (i.e., the lowest retention time) observed in the oligosaccharide analysis.

“Secondary prevention” means, in an individual who has a condition or who has had a condition, (i) prevention of reoccurrence of the condition, and/or (ii) increasing the duration of remission of the condition.

“Short chain fatty acid” includes butyrate, propionate, betahydroxybutyrate, lactate, acetate, or any combination thereof.

“Specified reaction time” or “reaction time” means the time for a reaction to proceed toward an equilibrium state between reagents added and products produced by the reaction of the reagents. In certain aspects, specified reaction time allows sufficient time to reach an equilibrium. In certain other aspects, specified reaction time, while allowing time for the reaction to proceed toward equilibrium, does not provide the time needed to reach equilibrium

“Soy” means any part of the plant in the genus Glycine or soja. The plant may be Dolichos soja L., Glycine angustifolia Miq., Glycine gracilis Skvortsov, Glycine hispida (Moench) Maxim., Glycine soja, Phaseolus max L., Soja angustifolia, Soja hispida Moench, Soja japonica Savi, Soja max, Soja soja H., Soja viridis or other species. The plant may be other non-Glycine or Soja genus, which are colloquially known as soybean, kongbiji or soya. Soy may refer to by-products of the plant during harvest or food processing. Non-limiting examples include, Soy root, soy stem, soy leaves, soy flowers, soy fruiting pods, soybean, soy protein, soy okra (pulp or curd), soy fiber or soybean testa. Soy may refer to the solid material after roasting, fermentation, hot-water, enzymatic, chemical, alkaline, super critical fluid, sun drying, organic solvent, acidic, mechanical pressure or pressure-based extractions.

“Strong-Arrhenius base” means a compound that completely dissociates in water to release one or more hydroxide ions into solution. Examples include KOH, NaOH, Ba(OH)₂, CsOH, Sr(OH)₂, Ca(OH)₂, LiOH, and RbOH.

The term “subject” as used herein generally refers to a living organism suffering from (e.g., exhibiting symptoms of) or prone to a disease or condition that can be prevented or treated by administration of a compound, nutritional formulation or supplement, or medicament or pharmaceutical composition, as provided herein. Non-limiting examples include humans, other mammals, bovines, rats, mice, dogs, monkeys, goat, sheep, cows, deer, and other non-mammalian animals. In some embodiments, the subject is a human at risk of developing symptoms associated with disrupted circadian rhythm. In some embodiments, the subject is a human exhibiting disrupted gut barrier function. In some embodiments, a subject is a patient who has been diagnosed as having a particular disorder. In some embodiments, a subject is a human. In some embodiments, a subject is a human patient. In those aspects where the subject is a human, the human can be a pediatric or adult human including an elderly adult human. In those aspects where the patient is a human, the patient can be a pediatric or adult patient including an elderly adult human. In some embodiments, the patient is a human at risk of developing symptoms associated with disrupted circadian rhythm. In some embodiments, the patient is a human exhibiting disrupted gut barrier function. In some embodiments, a patient is a mammal. In some embodiments, a patient is a mouse. In some embodiments, a patient is an experimental animal. In some embodiments, a patient is a rat. In some embodiments, a patient is a test animal. In some embodiments, a subject is a domesticated animal. In some embodiments, a subject is a farm animal. In some embodiments, a subject is a pet. In some embodiments, a patient is a farm animal or a pet.

“Substantially commensurate with initiation of peroxide-quenching” means the relationship between the timing of a cleavage reaction and the timing of a peroxide quenching reaction indicating that the initiation of the cleavage reaction and the initiation of the peroxide quenching reaction occur within a short time duration of each other (e.g. on the order of seconds, or on the order of minutes but not more than one day).

“Subunit” means a species that is covalently bonded to or within an oligomer (e.g., oligosaccharide) or polymer (e.g., polysaccharide). Such species generally can include saccharides (e.g., glucose, galactose, mannose, etc.). For example, when an oligosaccharide composition comprises a glucose subunit, it means that the composition comprises a glucose molecule that is bound to or within an oligomer or polymer. Therefore a composition that contains only free monomeric glucose would not contain a glucose subunit. Similarly, when an oligosaccharide composition comprises a sum of glucose, galactose, and mannose subunits in an amount of at least 60 wt. % based on total weight of saccharide subunits, this means that the mass of all of the glucose subunits, galactose subunits, and mannose subunits are summed, and the subunits of all saccharides are summed, and then the first sum is divided by the second sum. Additionally, when an oligosaccharide composition comprises non-terminal galactose subunits, and at least 70 wt. % of the non-terminal galactose subunits are specified to have at least one 4-linkage, this feature is calculated by summing the mass of all non-terminal galactose subunits having at least one 4-linkage (and this can include, for example, galactose subunits with 4,6-linkages and 4,3-linkages), and then dividing by the total mass of non-terminal galactose subunits regardless of linkage type. The same concept is applicable to any feature where reference to “at least one X-linkage,” in which X is an integer (e.g., such as “a weight ratio of glucose subunits having at least one 4-linkage to glucose subunits having at least one 3-linkage is between 2:1 to 4:1” and other such features). Moreover, in such calculations the actual mass of the subunit is used (i.e., in bound form) rather than the mass of the unit as if it was hydrolyzed (which would add the mass of water). Other features can be calculated similarly. These features can be determined with the aid of the various analytical techniques, such as hydrolytic monosaccharide compositional analysis, oligosaccharide analysis, glycosidic linkage analysis, NMR HSQC Analysis, and so forth, as well as other techniques known in the art.

“Treat” means to address a condition or disease with the objective of improving or stabilizing an outcome in the person being treated or addressing an underlying need. Treat therefore includes the nutritional management of the condition or disease by addressing nutritional needs of the person being treated. “Treating” and “treatment” have grammatically corresponding meanings.

“Treated polysaccharide” means a polysaccharide which has been contacted with at least one reagent capable of reacting with the polysaccharides (e.g. an enzyme or a Fenton's reagent).

“Weak-Arrhenius base” means a compound that incompletely dissociates in water to release one or more hydroxide ions into solution, e.g. ammonium hydroxide, H₂O, etc. There are no compounds which meet both the definitions used of strong-Arrhenius base and weak-Arrhenius base.

“Yeast beta glucan” means a beta glucan found in the cell walls of yeast. The polysaccharide of yeast beta glucan” contains beta-linked glucose units that may be in the beta-3 position, the beta-4 position, or the beta-6 position. Yeast beta glucan may be found alongside other polymers such as mannans. The yeast beta glucan can have a structure in which the backbone is beta-3 linked and the beta-6 linkages are long branches. Yeast beta glucan can be derived from Saccharomyces cerevisiae or other yeast within or outside of the Saccharomyces genus. Yeast beta glucan can refer to the solid material after roasting, fermentation, hot-water, enzymatic, chemical, alkaline, super critical fluid, sun drying, organic solvent, acidic, mechanical pressure or pressure-based extractions.

In this specification, when an amount of a component is expressed in terms of weight or mole percent, it is intended that the amount is on a dry basis unless otherwise specified. Dry basis means the absence of water or other solvent. For example, when a composition comprises 10 g of glucose, 40 g of xylose, and 50 g of water, it means the composition comprises 25% (mass % or wt. %) glucose on a dry basis, but the glucose is present in the composition at a concentration of 10% (mass % or wt. %).

One aspect of the invention provides a method of managing a disorder associated with disrupted circadian rhythm in a subject, the method comprising administering to the subject an effective amount of a beta glucan and/or an arabinan oligosaccharide.

In an embodiment, the beta glucan oligosaccharide and/or arabinan oligosaccharide is produced by reacting a beta glucan polysaccharide or an arabinan polysaccharide, respectively, in a reaction mixture with a Fenton's reagent in the form of a peroxide agent and a metal ion, to provide a treated polysaccharide. The treated polysaccharide can then be cleaved with a base to generate a mixture of polysaccharide cleavage products and/or oligosaccharides characteristic of the beta glucan or the arabinan polysaccharide that was treated. In embodiments, the arabinan polysaccharide is a legume polysaccharide and the resulting arabinan oligosaccharide is also designated a legume oligosaccharide or a legume fiber oligosaccharide. Suitable Fenton-type depolymerizations are described in patent applications WO2021097138A1, WO2018236917A1, WO2020247389A1, WO2022241163A1 and WO2023220318, the disclosures of each of which is incorporated by reference herein in its entirety to the extent not inconsistent with the disclosures herein.

In an embodiment, the Fenton's reagent comprises hydrogen peroxide, and one or more metal ions selected from the transition metals Fe(II), Fe(III), Cu(I), Cu(II), Mn(II), Zn(II), Ni(II), and Co(II), the alkaline earth metals Ca(II) and Mg(II), and the lanthanide Ce(IV). In an embodiment, the hydrogen peroxide concentration is about 1% to about 7%, for example about 3.5 to 4.5% (v/v). In an embodiment, the hydrogen peroxide concentration is about 4.0 (v/v). In an embodiment the metal ion is a copper ion; for example, Cu (II). In an embodiment, Cu (II) is used in the reaction mixture at a concentration of about 0.25 mM to about 1.00 mM, for example at a concentration of about 0.7 mM to about 0.8 mM. In an embodiment, Cu (II) is used at a concentration of about 0.75 mM. In an embodiment, the source of Cu (II) is copper sulfate.

In an embodiment, the base is selected from ammonium hydroxide, ammonium bicarbonate, ammonia, urea, sodium amide, dimethyl amine, trimethylamine, pyridine, and N,N-diisopropylethylamine, sodium hydroxide, calcium hydroxide, potassium hydroxide, barium hydroxide, and/or lithium hydroxide. For example, the base can be ammonium hydroxide and/or sodium hydroxide. In an embodiment, the base is a nitrogen-based cleavage reagent. In an embodiment, the nitrogen-based cleavage reagent is also a peroxide quenching reagent, and initiation of polysaccharide cleavage is commensurate, or substantially commensurate with initiation of peroxide-quenching. In an alternative embodiment, the nitrogen-based cleavage agent is not a peroxide-quenching agent, and the method further comprises initiation of peroxide quenching with an additional agent that is a peroxide-quenching agent. In an embodiment, the base concentration is about 0.2 M to about 1M, for example about 0.3 M to about 0.5M. In an embodiment, the base concentration is about 0.4 M. In an embodiment, the base is added to a final pH of about 8.5 to 11, for example to a final pH of about 9.5 to 10.5. In an embodiment, the base is added to a final pH of about 10. In an embodiment, the polysaccharide loading in the reaction mixture is between about 2% and about 20% (w/v), for example between about 8% and about 12% (w/v). In an embodiment, the polysaccharide loading is about 10% (w/v) in the reaction mixture.

The beta glucan oligosaccharide and/or arabinan oligosaccharide is optionally isolated or optionally substantially purified. In some aspects, a substantially purified oligosaccharide has a chemical purity of 95% by mass, optionally for some applications 99% by mass, optionally for some applications 99.9% by mass, optionally for some applications 99.99% by mass, and optionally for some applications 99.999% by mass.

In an embodiment, the beta glucan polysaccharide is derived from a grain, for example from oat or barley. In an embodiment, the beta glucan polysaccharide is, for example, a beta glucan having a weight average molecular weight of 500 kDa or more.

In an embodiment, the arabinan polysaccharide is an arabinan or arabinogalactan. In embodiments, the arabinan or arabinogalactan is a legume polysaccharide derived from a legume, for example from pea or soy. In an embodiment, the legume polysaccharide is an arabinan or arabinogalactan, particularly one having a weight average molecular weight of 500 kDa or more.

In an embodiment, the beta glucan and/or arabinan oligosaccharide has a dynamic viscosity ranging from about 1 to about 10 mPa s (Pascal second) at 100 mg/ml at 25° C. In an embodiment, the beta glucan oligosaccharide has a dynamic viscosity ranging from about 1 to about 5 mPa s at 100 mg/ml at 25° C. In an embodiment, the beta glucan oligosaccharide has a dynamic viscosity ranging from about 1 to about 3 mPa s at 100 mg/ml at 25° C. In embodiments, the beta glucan and/or arabinan oligosaccharide has a dynamic viscosity ranging from about 1 to about 1.5 mPa s g/ml at 25° C. In an embodiment, the beta glucan and/or arabinan oligosaccharide has a dynamic viscosity of about 1.3 to about 1.4 mPa s at 100 mg/ml at 25° C. Any viscosity measurement employs water as the solvent, unless specified otherwise.

The beta glucan and/or arabinan oligosaccharide can be formulated into any suitable form, for example a nutritional formula, a medicament, and the like. The nutritional formula can take any form that is suitable for human consumption. For example, the nutritional formula may be a food, a dietary supplement, a complete nutritional formula, a nutritional supplement, a nutraceutical, a powdered nutritional product which is to be reconstituted in water or milk before consumption, a beverage or a drink.

If the nutritional formula containing the oligosaccharide or mixture of oligosaccharides is formulated to contain a source of energy (for example as a food, a complete nutritional formula, a nutritional supplement, and the like), the nutritional formula can contain a source of protein. The source of protein can be in the form of intact protein, partially hydrolyzed protein, extensively hydrolyzed protein, or amino acids. The protein source can be any source of protein which is suitable for human consumption and particularly in adult nutritional formulations, suitable for consumption by adults, for example, cow's milk protein, goat's milk protein, rice protein, pea protein, soya protein, and the like. The milk protein is optionally in the form of whey protein, casein, or combinations of whey and casein. The whey protein can take many forms such as whey protein concentrates, whey protein isolates, whey protein micelles, whey protein hydrolysates, acid whey, sweet whey, modified sweet whey (sweet whey from which the caseino-glycomacropeptide has been removed), a fraction of whey protein, and any combination thereof. The protein source can be supplemented with free amino acids. Suitable amino acids include histidine, isoleucine, leucine, lysine, methionine, phenylalanine, threonine, tryptophan, valine, arginine, cysteine, and glutamine.

The protein content of the nutritional formula can be in the range of about 1.0 g/100 kcal to about 20.0 g/100 kcal, for example about 3.0 g/100 kcal to about 15 g/100 kcal. For certain adults, such as those suffering from severe muscle atrophy, the protein content can be higher than needed for healthy adults; for example the protein can provide at least 25% of the energy of the formula. In an embodiment, the nutritional formula can be formulated to deliver a daily dose of protein greater than 1.0 g protein/kg body weight/day, preferably greater than 1.2 g protein/kg body weight/day; for example up to 2.5 g protein/kg body weight/day. The daily dose of the protein can be provided by one or more servings of the nutritional formula per day.

The nutritional formula can also contain a source of digestible carbohydrate. Examples of digestible carbohydrates include lactose, maltose, sucrose, glucose, glucose syrup or dried glucose syrup, maltodextrins, and starch. Mixtures of carbohydrates can also be used. The carbohydrate source preferably contains little or no lactose.

The carbohydrate content of the nutritional formula can be in the range of about 5 g/100 kcal to about 20 g/100 kcal, for example about 7 g/100 kcal to about 15 g/100 kcal. The carbohydrates preferably do not provide greater than 50 energy % of the nutritional formula, more preferably not greater than 35 energy % of the nutritional formula.

The nutritional formula can further contain a source of lipid. The lipid source may be any lipid which is suitable for use in nutrition, particularly adult nutrition. Suitable lipid sources include milk fat, sunflower oil, rapeseed oil, safflower oil, egg yolk lipid, olive oil, coconut oil, palm oil, palm kernel oil, soybean oil, fish oil, and microbial fermentation oil containing long chain poly unsaturated fatty acids. These oils may be in the form of high oleic forms such as high oleic sunflower oil and high oleic safflower oil. The lipid source may also be in the form of fractions derived from these oils such as palm olein, medium chain triglycerides (MCT), and esters of fatty acids such as linoleic acid, palmitic acid, stearic acid, linolenic acid, oleic acid, lauric acid, capric acid, caprylic acid, caproic acid, and the like. The lipid source can also include structured lipids (i.e., lipids that are modified chemically or enzymatically to change their structure). Preferably, the structured lipids are SN2-structured lipids, for example comprising triglycerides having an elevated level of palmitic acid at the SN2 position of the triglyceride. The lipid source can also include oils containing high concentrations of long-chain, polyunsaturated fatty acids such as arachidonic acid (ARA), docosahexaenoic acid (DHA), and/or eicosapentaenoic acid such as fish oils or microbial oils. If the nutritional formula is intended for an adult suffering from neurocognitive decline, in an embodiment the lipid source preferably contains MCT. For example, the lipid source can contain up to 40% by weight of MCT, preferably about 15% to about 35% by weight of the lipid source.

The lipid content of the nutritional formula can be in the range of about 0.5 g/100 kcal to about 5 g/100 kcal, for example about 1 g/100 kcal to about 3 g/100 kcal.

When in nutritionally complete form, the nutritional formula contains all vitamins and minerals understood to be essential in the daily diet and in nutritionally adequate amounts. Minimum requirements have been established for certain vitamins and minerals. Minerals which are normally required include sodium, potassium, chloride, calcium, phosphorous, magnesium, iron, zinc, copper, iodine, selenium, manganese, molybdenum, and fluoride. In general, the molar ratio of calcium to available phosphorus is about 1:1 to about 2:1. Vitamins which are normally required include vitamin A, vitamin D, thiamine (vitamin B1), riboflavin (vitamin B2), niacin (vitamin B3), pantothenic acid (vitamin B5), vitamin B6, biotin (vitamin B7), folate (vitamin B9), vitamin B12, vitamin C, vitamin E, and vitamin K. The nutritional formula may further contain one or more carotenoids.

The nutritional formula can contain sources of other essential nutrients such as choline. Suitable sources of choline include milk fat, milk fat fractions, phospholipids, and choline salts. In general, the nutritional formula can contain about 7 mg/100 kcal to about 50 mg/100 kcal. The nutritional formula can also contain a source of inositol. For example, the formula may contain about 4 mg/100 kcal to about 150 mg/100 kcal.

The nutritional formula can also comprise at least one probiotic. If the probiotic is capable of producing lactic acid, a probiotic which produces L(+) lactic acid is preferred. Examples of probiotics include yeasts, such as Saccharomyces; and bacteria, such as the genera Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus and Lactobacillus. Specific examples of suitable probiotics are: Saccharomyces cerevisiae, Bacillus coagulans, Bacillus licheniformis, Bacillus subtilis, Bifidobacterium bifidum, Bifidobacterium infantis, Bifidobacterium longum, Bifidobacterium breve, Enterococcus faecium, Enterococcus faecalis, Lactobacillus acidophilus, Lactobacillus alimentarius, Lactobacillus casei subsp. casei, Lactobacillus casei Shirota, Lactobacillus curvatus, Lactobacillus delbrueckii subsp. lactis, Lactobacillus farciminis, Lactobacillus gasseri, Lactobacillus helveticus, Lactobacillus johnsonii, Lactobacillus rhamnosus (Lactobacillus GG), Lactobacillus sake, Lactococcus lactis, Lactobacillus reuterii, Micrococcus varians, Pediococcus acidilactici, Pediococcus pentosaceus, Pediococcus acidilactici, Pediococcus halophilus, Streptococcus faecalis, Streptococcus thermophilus, Staphylococcus carnosus and Staphylococcus xylosus.

The nutritional formula can contain other substances which may be beneficial to the subject, such as cholesterol, lactoferrin, nucleotides, nucleosides, sphingomyelin, choline and the like.

If desired, the nutritional formula can contain emulsifiers and stabilizers such as soy lecithin, citric acid esters of mono- and di-glycerides, and the like. This is especially the case if the nutritional formula is provided in liquid form.

The nutritional formula can be prepared in any suitable manner. For example, a nutritional formula can be prepared by blending the protein source, the carbohydrate source, and the lipid source in appropriate proportions. If used, emulsifiers may be included in the blend. The vitamins and minerals may be added at this stage, but are usually added later to avoid thermal degradation. Any lipophilic vitamins. emulsifiers and the like may be dissolved into the lipid source prior to blending. The ganglioside composition can be included in the lipid source prior to blending. Water, preferably water which has been subjected to reverse osmosis, may then be mixed in to form a liquid mixture.

The liquid mixture then can be thermally treated to reduce bacterial loads. For example, the liquid mixture may be rapidly heated to a temperature in the range of about 80° C. to about 110° C. for about 5 seconds to about 5 minutes. This may be carried out by steam injection or by heat exchanger, for example a plate heat exchanger.

The liquid mixture then can be cooled to about 60° C. to about 85° C., for example by flash cooling. The liquid mixture then can be homogenized, for example in two stages at about 7 MPa to about 40 MPa in the first stage and about 2 MPa to about 14 MPa in the second stage. The homogenized mixture then can be further cooled to add any heat sensitive components, such as vitamins and minerals. The pH and solids content of the homogenized mixture is conveniently standardized at this point.

If it is desired to produce a powdered nutritional formula, the homogenized mixture is transferred to a suitable drying apparatus such as a spray drier or freeze drier and converted to powder. The powder preferably has a moisture content of less than about 5% by weight.

If it is desired to produce a liquid nutritional formula, the homogenized mixture is filled into suitable containers, preferably aseptically. However, the liquid nutritional formula also can be retorted in the container. Suitable apparatus for carrying out filling of this nature is commercially available. The liquid nutritional formula may be in the form of a ready to feed formula having a solids content of about 10 to about 14% by weight or may be in the form of a concentrate, usually having a solids content of about 20 to about 26% by weight.

The nutritional formula can also be formulated as an incomplete nutritional formula which is intended to supplement the human diet. In such a case, the nutritional formula can contain reduced amounts, or none, of the protein, lipid, carbohydrate, vitamins and minerals defined above.

The nutritional formula can also be formulated as a supplement in a unit dosage form containing a unit dose of the beta glucan and/or arabinan oligosaccharide. The supplement can contain an acceptable food-grade carrier, e.g. phosphate buffered saline solution, mixtures of ethanol in water, water and emulsions such as an oil/water or water/oil emulsion, as well as various wetting agents or excipients. The supplement can also contain other excipients that do not produce an adverse, allergic or otherwise unwanted reaction when administered to a human. The excipients can include solvents, dispersants, coatings, absorption promoting agents, controlled release agents, and one or more inert excipients, such as starches, granulating agents, microcrystalline cellulose, diluents, lubricants, binders, and disintegrating agents.

The supplement can be formulated to be administered orally, e.g. as a tablet, capsule, or pellet containing a predetermined amount of the oligosaccharide. It can also be formulated as a powder or granules containing a predetermined amount of the synthetic complex lipid or a gel, paste, solution, suspension, emulsion, syrup, bolus, electuary, or slurry, in an aqueous or non-aqueous liquid, containing a predetermined concentration of the synthetic complex lipid. The supplement can include one or more binders, lubricants, inert diluents, flavoring agents, and humectants. When in a form such as a tablet, the supplement can be coated and can be formulated to provide sustained, delayed or controlled release of the synthetic complex lipid. The supplement can also include active agents such as vitamins, minerals, probiotics, and the like as described above.

As is understood in the art, the presence and amounts of components of nutritional formula or supplements can be adapted for consumption by various subjects dependent upon age, weight, health, the presence of disease or disorders, among other factors. One of ordinary skill in the art can readily adapt nutritional formula and supplements for such varied applications using methods that well known in the art in view of the descriptions herein. In preferred embodiments, the nutritional formulations and supplements are intended for consumption by adult humans and in certain embodiments for consumption by elderly adult humans. It will be appreciated by one of ordinary skill in the art that the nutritional formulations, nutritional supplements and medicaments herein can also be employed with animals other than humans, and in particular are useful for the management and treatment of symptoms as described herein in non-human animals. The formulations and methods herein have particular application for veterinary application, such as for use in domesticated animals, including farm animals and pets.

The amount of the beta glucan and/or arabinan oligosaccharide required to be administered will vary depending upon factors such as the risk and severity of the disorder, any underlying medical condition or disease, age, the delivery form of the oligosaccharide, and other medications being administered. However, the required amount can be readily set by a person of ordinary skill in the art and would generally be in the range from about 500 mg to about 50 g per day, in certain embodiments from about 1 g to about 20 g per day, for example from about 2 g to about 10 g per day, about 3 g to about 6 g per day. An appropriate dose can be determined based on several factors, including, for example, body weight and/or condition, the severity of the disorder, other ailments and/or diseases, the incidence and/or severity of side effects and the manner of administration. Appropriate dose ranges may be determined by methods known to those skilled in the art.

For the primary prevention of the subject developing symptoms associated with disrupted circadian rhythm, the subject is ideally assessed to determine whether the subject is at risk of developing symptoms associated with disrupted circadian rhythm. In an embodiment, this may be carried out by assessing the intestinal barrier functioning of the subject. If the intestinal barrier of the subject is compromised, the subject is at risk of developing symptoms associated with disrupted circadian rhythm. This may be determined by methods known to those skilled in the art, for example, in an embodiment, the intestinal barrier function of the subject is assessed using one or more of lactulose and mannitol permeability, blood zonulin levels, dynamic contrast-enhanced magnetic resonance imaging, histology and confocal laser endomicroscopy.

The duration of the administration of the beta glucan and/or arabinan oligosaccharide will vary depending upon factors such as the risk and severity of the disorder, age, the form of the composition, the dose and other medications being administered. However, the duration can be readily set by a medical practitioner. For example, the beta glucan and/or arabinan oligosaccharide can be administered for a period of at least about 14 days, at least about 1 month, at least about 6 months, at least about 1 year, or chronically for the rest of the adult's life. The beta glucan and/or Arabinan oligosaccharide can be administered daily, or with intervals between administrations longer than a day. Further, the oligosaccharide can be administered more than once a day.

The administration of the beta glucan and/or arabinan oligosaccharide can be combined with exercise because appropriate exercise is known to help maintain circadian rhythm. The extent of the exercise can be readily determined by a health care practitioner, taking into account the condition of the subject.

The oligosaccharides and oligosaccharide compositions disclosed herein can be formulated into a variety of formulations or compositions, and such formulations or compositions can be administered to a subject (e.g., a patient, mammal, human, etc.) in a variety of ways. In some aspects, the oligosaccharides and oligosaccharide compositions can be formulated, for example, into a nutritional composition, a nutritional supplement, a pharmaceutical composition, or other composition or formulation. The oligosaccharides and oligosaccharide compositions can be administered by another person to the subject or patient (e.g., orally, intravenously, and/or topically) or they can be self-administered by the subject or patient (e.g., orally, such as via tablets or capsules, and/or topically via a cream, ointment, or gel). Administration of one or more of the oligosaccharides and oligosaccharide compositions disclosed herein can be in combination with other compounds, such as excipients, colorants, fillers, binders, diluents, buffering agents, moistening agents, preservatives, flavoring agents, dyes, disintegrating agents, pharmaceutically compatible carriers, or any combination thereof, is contemplated.

The oligosaccharide or oligosaccharide composition may take any form (e.g., as a formulation) which is suitable for delivery of the oligosaccharide into the gastrointestinal tract (including the stomach and rectum) of the subject. Suitable forms include enterally administered nutritional compositions, orally administered unit dosage forms, buccally administered unit dosage forms, and rectally administered unit dosage forms. The enterally administered nutritional compositions may be suitable for administration through a nasogastric tube, through a jejunum tube, orally, and the like. The enterally administered nutritional composition may be suitable for administration through a nasogastric tube, through a jejunum tube, orally, and the like. In embodiments, the enterally administered nutritional composition can include other components of nutritional value and can be formulated as a soluble powder, a liquid concentrate, a ready-to-use formulation, a food, a snack, and the like. In embodiments, the orally administered unit dosage form can be a tablet, a capsule, a pellet, a powder, a gel, a paste, a solution, a suspension, an emulsion, a syrup, a liquid, and the like. The orally administered unit dosage form can be coated and/or formulated to provide sustained, delayed or controlled release of the oligosaccharide, and can contain other active components. The orally administered unit dosage form can be formulated for pharmaceutical use, dietary supplement use or nutritional use. The buccally administered unit dosage form is conveniently in the form of a tablet, pellet, wafer, film, patch, spray, drop or gel suitable for delivery into the buccal cavity, include for sublingual delivery. The rectally administered unit dosage form is conveniently a suppository, a capsule, a tablet, an enema, a gel, a foam, a cream and the like. The buccally and rectally administered dosage forms can include other active components.

In embodiments, the oligosaccharide or oligosaccharide compositions can be formulated into pills or tablets or encapsulated in capsules, such as gelatin capsules. Tablet forms can optionally include, for example, one or more of lactose, sucrose, mannitol, sorbitol, calcium phosphates, corn starch, potato starch, alginic acid, microcrystalline cellulose, acacia, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge or candy forms can comprise the compositions in a flavor, e.g., sucrose, as well as pastilles comprising the compositions in an inert base, such as gelatin and glycerin or sucrose and acacia emulsions, gels, and the like containing, in addition to the active ingredient, carriers known in the art. The prebiotic or probiotic oligosaccharide containing formulations may also contain conventional food supplement fillers and extenders such as, for example, rice flour. The nutritional composition can also be in a unit dosage form or as a pharmaceutical composition. The unit dosage form can contain an acceptable food-grade carrier, e.g., phosphate buffered saline solution, mixtures of ethanol in water, water and emulsions such as an oil/water or water/oil emulsion, as well as various wetting agents or excipients. The unit dosage form can also contain other materials that do not produce an adverse, allergic, or otherwise unwanted reaction when administered to a subject. The carriers and other materials can include solvents, dispersants, coatings, absorption promoting agents, controlled release agents, and one or more inert excipients, such as starches, granulating agents, microcrystalline cellulose, diluents, lubricants, binders, and disintegrating agents. Preferably carriers and other materials are low in FODMAPs or contain no FODMAPs.

In embodiments, the formulation comprising oligosaccharides or oligosaccharide compositions described herein is in the form of a pharmaceutical composition. Pharmaceutical compositions herein comprise a named active ingredient (e.g., beta glucan oligosaccharide and/or arabinan oligosaccharide) in an amount effective for achieving the desired biological or therapeutic activity for a given form of administration to a given subject or patient and optionally contain a pharmaceutically acceptable carrier. Pharmaceutical compositions can include an amount (for example, a unit dosage) of one or more of the disclosed oligosaccharides or other active ingredient together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, Pa. (19th Edition).

Pharmaceutically acceptable carriers are those carriers that are compatible with the other ingredients in the formulation and are biologically acceptable. Carriers can be solid or liquid. It is currently contemplated that preferred carrier are liquid carriers. Carriers can include one or more substances that can also act as solubilizers, suspending agents, fillers, glidants, compression aids, binders, tablet-disintegrating agents, or encapsulating materials. Liquid carriers can be used in preparing solutions, suspensions, emulsions, syrups and elixirs. The active ingredient can be dissolved or suspended in a pharmaceutically acceptable liquid carrier such as water (of appropriate purity, e.g., pyrogen-free, sterile, etc.), an organic solvent, a mixture of both, or a pharmaceutically acceptable oil or fat. The liquid carrier can contain other suitable pharmaceutical additives such as, for example, solubilizers, emulsifiers, buffers, preservatives, sweeteners, flavoring agents, suspending agents, thickening agents, colors, viscosity regulators, stabilizers or osmo-regulators. Compositions for oral administration can be in either liquid or solid form.

Suitable examples of liquid carriers for oral and parenteral administration include water of appropriate purity, aqueous solutions (particularly containing additives, e.g. cellulose derivatives, sodium carboxymethyl cellulose solution), alcohols (including monohydric alcohols and polyhydric alcohols e.g. glycols) and their derivatives, and oils. Sterile liquid carriers are used in sterile liquid form compositions for parenteral administration and can include water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants. Liquid pharmaceutical compositions that are sterile solutions or suspensions can be administered by, for example, intramuscular, intraperitoneal or subcutaneous injection. Sterile solutions can also be administered intravenously. Compositions for oral administration can be in either liquid or solid form. The carrier can also be in the form of creams and ointments, pastes, and gels. The creams and ointments can be viscous liquid or semisolid emulsions of either the oil-in-water or water-in-oil type.

Oils, which can be used in parenteral formulations include petroleum, animal, vegetable, or synthetic oils. Specific examples of oils include peanut, soybean, sesame, cottonseed, com, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

Nutritional formulations, nutritional supplements and pharmaceutical compositions can be in the form of a unit dosage which is the amount of an active ingredient administered to a subject or patient in a single dose. The unit dosage form can be administered orally, e.g., as a tablet, capsule, or pellet containing a predetermined amount of the mixture, or as a powder or granules containing a predetermined concentration of the mixture or a gel, paste, solution, suspension, emulsion, syrup, bolus, electuary, or slurry, in an aqueous or non-aqueous liquid, containing a predetermined concentration of the mixture. An orally administered composition can include one or more binders, lubricants, inert diluents, flavoring agents, and humectants. An orally administered composition such as a tablet can optionally be coated and can be formulated to provide sustained, delayed, or controlled release of the oligosaccharide compositions. The unit dosage form can also be administered by rectal suppository, aerosol tube, naso-gastric tube or direct infusion into the GI tract or stomach. The unit dosage form can also include agents such as antibiotics, probiotics, analgesics, and anti-inflammatory agents. An effective amount of an active ingredient, such as the oligosaccharides or oligosaccharide compositions herein, can be provided in one or more than one unit dosage form.

The proper dosage of the unit dosage form, pharmaceutical composition, the nutritional formulation or nutritional supplement can be determined in a conventional manner, based upon factors such as the subject's condition, immune status, body weight and age.

Various aspects are contemplated herein, several of which are set forth in the paragraphs below. It is explicitly contemplated that any aspect or portion thereof can be combined to form an aspect. In addition, it is explicitly contemplated that any aspect (e.g., Aspect A13) that references an aspect (e.g., Aspect A1) for which there are sub-aspects having the same top level number (e.g., Aspect A1a, A1b, A1c, and so forth) necessarily includes reference to those sub-aspects A1a, A1b, A1c, and so forth. In other words, if Aspect A13 refers to Aspect A1, and there are Aspects A1a and Alb present, then Aspect A13 refers to Aspects A1a or A1b. Furthermore, although the aspects below are subdivided into aspects A, B, C, D, and so forth, it is explicitly contemplated that aspects in each of subdivisions A, B, C, D, etc. can be combined in any manner. Moreover, the term “any preceding aspect” means any aspect that appears prior to the aspect that contains such phrase (in other words, the sentence “Aspect B13: The method of any one of aspects B1-B12, or any preceding aspect,” means that any aspect prior to aspect B13 is referenced, including aspects B1-B12 and all of the “A” aspects). For example, it is contemplated that, optionally, any method or medicament of any of the below aspects may be useful with or combined with any other aspect provided below. Further, for example, it is contemplated that any embodiment described elsewhere herein, including above this paragraph, may optionally be combined with any of the below listed aspects. In some instances in the aspects below, or elsewhere herein, two open ended ranges are disclosed to be combinable into a range. For example, “at least X” is disclosed to be combinable with “less than Y” to form a range, in which X and Y are numeric values. For the purposes of forming ranges herein, it is explicitly contemplated that “at least X” combined with “less than Y” forms a range of X-Y inclusive of value X and value Y.
Aspect A1: A method of reducing the severity of symptoms associated with disrupted circadian rhythm in a subject, the method comprising, consisting essentially of, or consisting of administering to the subject an effective amount of a beta-glucan oligosaccharide and/or an arabinan oligosaccharide.
Aspect B1: A method of treating symptoms associated with disrupted circadian rhythm in a subject, the method comprising, consisting essentially of, or consisting of administering to the subject an effective amount of a beta-glucan oligosaccharide and/or an arabinan oligosaccharide.
Aspect C1: A method for the primary prevention of symptoms associated with disrupted circadian rhythm in a subject, the method comprising, consisting essentially of, or consisting of administering to the subject an effective amount of a beta-glucan oligosaccharide and/or an arabinan oligosaccharide.
Aspect C2: The method of aspect C1, or any preceding aspect, further comprising determining whether the subject is at risk of developing the symptoms associated with disrupted circadian rhythm, prior to administering the beta-glucan oligosaccharide and/or the arabinan oligosaccharide. In a related aspect, the method of aspect C1 or any preceding aspect, consisting essentially of administering to the subject an effective amount of a beta-glucan oligosaccharide and/or an arabinan oligosaccharide and determining whether the subject is at risk of developing the symptoms associated with disrupted circadian rhythm, prior to administering the beta-glucan oligosaccharide and/or the arabinan oligosaccharide. In a related aspect, the method of aspect C1 or any preceding aspect, consisting of administering to the subject an effective amount of a beta-glucan oligosaccharide and/or an arabinan oligosaccharide and determining whether the subject is at risk of developing the symptoms associated with disrupted circadian rhythm, prior to administering the beta-glucan oligosaccharide and/or the arabinan oligosaccharide.
Aspect C2a: The method of aspect C1 or aspect C2, or any preceding aspect, wherein determining whether the subject is at risk of developing the symptoms comprises assessing the intestinal barrier function of the subject.
Aspect C2b: The method of aspect C1 or aspect C2, or any preceding aspect, wherein the subject is assessed to be at risk of developing the symptoms associated with disrupted circadian rhythm.
Aspect C2c: The method of aspect C1 or aspect C2, or any preceding aspect, wherein the subject is assessed to be at risk of developing the symptoms associated with disrupted gut barrier function.
Aspect D1: A method for the secondary prevention of symptoms associated with disrupted circadian rhythm in a subject, the method comprising, consisting essentially of, or consisting of administering to the subject an effective amount of a beta-glucan oligosaccharide and/or an arabinan oligosaccharide.
Aspect E1: A method of delaying the progression of symptoms associated with disrupted circadian rhythm in a subject, the method comprising, consisting essentially of, or consisting of administering to the subject an effective amount of a beta-glucan oligosaccharide and/or an arabinan oligosaccharide.
Aspect F1: The method of any one of aspects A1-E1, or any preceding aspect, wherein the symptoms comprise sleep-related symptoms.
Aspect F2: The method of any one of aspects A1-F1, or any preceding aspect, wherein the symptoms comprise mood-related symptoms.
Aspect F3: The method of any one of aspects A1-F2, or any preceding aspect, wherein the symptoms comprise symptoms associated with cognitive functioning and neurocognitive decline.
Aspect F4: The method of aspect F3, or any preceding aspect, wherein the symptoms comprise a loss of executive function and/or a loss of memory. In a related aspect, of aspect F3, or any preceding aspect, wherein the symptoms are a loss of executive function and/or a loss of memory.
Aspect F5: The method of any one of aspects A1-F4, or any preceding aspect, wherein the effective amount of the beta-glucan oligosaccharide and/or the arabinan oligosaccharide ranges from about 0.5 g to about 50 g per day. For example, an effective amount of the beta-glucan oligosaccharide and/or the arabinan oligosaccharide range from about 0.5 g to about 50 g per day, about 0.5 g to about 40 g per day, about 0.5 g to about 30 g per day, about 0.5 g to about 20 g per day, about 0.5 g to about 10 g per day, about 0.5 g to about 7.5 g per day, about 0.5 g to about 5 g per day, about 0.75 g to about 50 g per day, about 0.75 g to about 40 g per day, about 0.75 g to about 30 g per day, about 0.75 g to about 20 g per day, about 0.75 g to about 10 g per day, about 0.75 g to about 7.5 g per day, about 0.75 g to about 5 g per day, about 1 g to about 50 g per day, about 1 g to about 40 g per day, about 1 g to about 30 g per day, about 1 g to about 20 g per day, about 1 g to about 10 g per day, about 1 g to about 7.5 g per day, about 1 g to about 5 g per day, about 5 g to about 50 g per day, about 5 g to about 40 g per day, about 5 g to about 30 g per day, about 5 g to about 20 g per day, about 5 g to about 10 g per day, or about 5 g to about 7.5 g per day.
Aspect F6: The method of any one of aspects A1-F5, or any preceding aspect, wherein the beta-glucan oligosaccharide contains, consists essentially of or consists of beta-1,3 and beta-1,4 linked glucose residues.
Aspect F7: The method of aspect F6, or any preceding aspect, wherein the beta-glucan oligosaccharide contains about 3 to about 50 subunits (e.g., about 3 to about 50, or about 3 to about 40, or about 3 to about 35, or about 3 to about 30, or about 3 to about 25 subunits, or about 5 to about 50, or about 5 to about 40, or about 5 to about 35, or about 5 to about 30, or about 5 to about 25 subunits) wherein each subunit is a beta-1,3 glucose residue and/or a beta-1,4 glucose residue.
Aspect F8: The method of aspect F6, or any preceding aspect, wherein the beta-glucan oligosaccharide comprises beta-1,3 linked glucose residues: beta-1,4 linked glucose residues in a ratio of 1:1 to 1:5; for example 1:1, or 1:2, or 1:3, or 1:4, or 1:5.
Aspect F9: The method of any one of aspects A1-F8, or any preceding aspect, wherein the beta-glucan oligosaccharide has an average molecular weight (Mw) of less than 10,000 Da (e.g., less than 10,000 Da, less than 9,000 Da, less than 8,000 Da, less than 7,500 Da, less than 7,000 Da, less than 6,000 Da, or less than 5,000 Da).
Aspect F10: The method of any one of aspects A1-F9, or any preceding aspect, wherein the beta-glucan oligosaccharide has an average molecular weight (Mw) of less than 8,000 Da (e.g., less than 8,000 Da, less than 7,500 Da, less than 7,000 Da, less than 6,000 Da, or less than 5,000 Da).
Aspect F11: The method of any one of aspects A1-F10, or any preceding aspect, wherein the beta-glucan oligosaccharide has a dynamic viscosity ranging from about 1 to about 10 mPa s at 100 mg/ml at 25° C. For example, the beta-glucan oligosaccharide has a dynamic viscosity ranging from about 1 to about 10 mPa s at 100 mg/ml at 25° C., from about 1 to about 5 mPa s at 100 mg/ml at 25° C. or from about 1 to about 3 mPa s at 100 mg/ml at 25° C. or from about 1 to about 1.5 mPa s at 100 mg/ml at 25° C. or from about 1.3 to about 1.4 mPa s at 100 mg/ml at 25° C.
Aspect F12: The method of any one of aspects A1-F11, or any preceding aspect, wherein at least 70% of the mass of the beta-glucan oligosaccharide has a molecular mass of less than 100 kDa (e.g., less than 100 kDa, less than 90 kDa, less than 80 kDa, less than 75 kDa, less than 70 kDa, less than 60 kDa, less than 50 kDa, less than 40 kDa, less than 30 kDa, less than 25 kDa, less than 20 kDa, less than 15 kDa, less than 10 kDa, less than 7.5 kDa, less than 5 kDa, less than 4 kDa, less than 3 kDa, less than 2.5 kDa, less than 2 kDa, or less than 1 kDa).
Aspect F13: The method of any one of aspects A1-F11, or any preceding aspect, wherein at least 60% of the mass of the beta-glucan oligosaccharide has a molecular mass of less than 50 kDa (e.g., less than 50 kDa, less than 40 kDa, less than 30 kDa, less than 25 kDa, less than 20 kDa, less than 15 kDa, less than 10 kDa, less than 7.5 kDa, less than 5 kDa, less than 4 kDa, less than 3 kDa, less than 2.5 kDa, less than 2 kDa, or less than 1 kDa).
Aspect F14: The method of any one of aspects A1-F11, or any preceding aspect, wherein at least 50% of the mass of the beta-glucan oligosaccharide has a molecular mass of less than 15 kDa (e.g., less than 15 kDa, less than 10 kDa, less than 7.5 kDa, less than 5 kDa, less than 4 kDa, less than 3 kDa, less than 2.5 kDa, less than 2 kDa, or less than 1 kDa).
Aspect F15: The method of any one of aspects A1-F11, or any preceding aspect, wherein at least 50% of the mass of the beta-glucan oligosaccharide has a molecular mass of less than 5 kDa (e.g., less than 5 kDa, less than 4 kDa, less than 3 kDa, less than 2.5 kDa, less than 2 kDa, or less than 1 kDa).
Aspect F16: The method of any one of aspects A1 to F5, or any preceding aspect, wherein the arabinan oligosaccharide is a legume oligosaccharide.
Aspect F17: The method of aspect F16, or any preceding aspect, wherein the arabinan oligosaccharide comprises alpha-1,5 linked arabinose residues, alpha-1.3 linked arabinose residues, alpha-1,2 linked arabinose residues, or any combination thereof.
Aspect F18: The method of aspect F16 or aspect F17, or any preceding aspect, wherein the alpha-linked arabinose residues are branched in the 2 and 3 positions, the 3 and 5 positions, or the 2 and 5 positions.
Aspect F19: The method of any one of aspects F16-F18, or any preceding aspect, wherein the alpha-linked arabinose residues are trisecting in the 2, 3, and 5 positions.
Aspect F20: The method of any one of aspects F16-F19, or any preceding aspect, wherein at least 80% (e.g., at least 80%, at least 85%, at least 90%, at least 95%, at least 99%, or 100%) of the arabinose residues consist of alpha-1.5 linked arabinose residues, alpha-1,3 linked arabinose residues, alpha-1,2 linked arabinose residues, or any combination thereof.
Aspect F21: The method of any one of aspects F16-F19, or any preceding aspect, wherein at least 70% (e.g., at least 70%, at least 75%, at least 80%, at least 85%, at least 90%, at least 95%, at least 99%, or 100%) of the mass of the arabinan oligosaccharide has a molecular mass of less than 100 kDa (e.g., less than 100 kDa, less than 90 kDa, less than 80 kDa, less than 75 kDa, less than 70 kDa, less than 60 kDa, less than 50 kDa, less than 40 kDa, less than 30 kDa, less than 25 kDa, less than 20 kDa, less than 15 kDa, less than 10 kDa, less than 7.5 kDa, less than 5 kDa, less than 4 kDa, less than 3 kDa, less than 2.5 kDa, less than 2 kDa, or less than 1 kDa).
Aspect F22: The method of any one of aspects F16-F19, or any preceding aspect, wherein at least 60% (e.g., at least 60%, at least 65%, at least 70%, at least 75%, at least 80%, at least 85%, at least 90%, at least 95%, at least 99%, or 100%) of the mass of the arabinan oligosaccharide has a molecular mass of less than 50 kDa (e.g., less than 50 kDa, less than 40 kDa, less than 30 kDa, less than 25 kDa, less than 20 kDa, less than 15 kDa, less than 10 kDa, less than 7.5 kDa, less than 5 kDa, less than 4 kDa, less than 3 kDa, less than 2.5 kDa, less than 2 kDa, or less than 1 kDa).
Aspect F23: The method of any one of aspects F16-F19, or any preceding aspect, wherein at least 40% (e.g., at least 40%, at least 45%, at least 50%, at least 55%, at least 60%, at least 65%, at least 70%, at least 75%, at least 80%, at least 85%, at least 90%, at least 95%, at least 99%, or 100%) of the mass of the arabinan oligosaccharide has a molecular mass of less than 15 kDa (e.g., less than 15 kDa, less than 10 kDa, less than 7.5 kDa, less than 5 kDa, less than 4 kDa, less than 3 kDa, less than 2.5 kDa, less than 2 kDa, or less than 1 kDa).
Aspect F24: The method of any one of aspects F16-F19, or any preceding aspect, wherein at least 20% (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%) of the mass of the arabinan oligosaccharide has a molecular mass of less than 5 kDa (e.g., less than 5 kDa, less than 4 kDa, less than 3 kDa, less than 2.5 kDa, less than 2 kDa, or less than 1 kDa).
Aspect F25: The method of any one of aspects F16-F24, or any preceding aspect, wherein the arabinan oligosaccharide contains about 3 to about 50 subunits (e.g., about 3 to about 50, or about 3 to about 40, or about 3 to about 35, or about 3 to about 30, or about 3 to about 25 subunits, or about 5 to about 50, or about 5 to about 40, or about 5 to about 35, or about 5 to about 30, or about 5 to about 25 subunits), wherein each subunit is an alpha-1,5 linked arabinose residues, an alpha-1,3 linked arabinose residues, or an alpha-1,2 linked arabinose residues.
Aspect F26: The method of any one of aspects F16-F25, or any preceding aspect, wherein the arabinan oligosaccharide is a legume oligosaccharide.
Aspect G1: Use of a beta glucan oligosaccharide and/or an arabinan oligosaccharide for reducing the severity of symptoms associated with disrupted circadian rhythm in a subject.
Aspect G2: Use of a beta glucan oligosaccharide for reducing the severity of symptoms associated with disrupted circadian rhythm in a subject.
Aspect G3: Use of an arabinan oligosaccharide for reducing the severity of symptoms associated with disrupted circadian rhythm in a subject.
Aspect G4: The use of any one of aspects G1-G3, or any preceding aspect, wherein the symptoms comprise sleep-related symptoms.
Aspect G5: The use of any one of aspects GT-G4, or any preceding aspect, wherein the symptoms comprise mood-related symptoms.
Aspect G6: The use of any one of aspects GT-G5, or any preceding aspect, wherein the symptoms comprise symptoms associated with cognitive functioning and neurocognitive decline.
Aspect G7: The use of any one of aspects G1-G6, or any preceding aspect, wherein the symptoms comprise a loss of executive function and/or a loss of memory. The use of any one of aspects GI-G6, or any preceding aspect, wherein the symptoms are a loss of executive function and/or a loss of memory.
Aspect H1: Use of a beta glucan oligosaccharide and/or an arabinan oligosaccharide for the primary prevention of symptoms associated with disrupted circadian rhythm in a subject, the method comprising, consisting essentially of, or consisting of administering to the subject an effective amount of a beta-glucan oligosaccharide and/or an arabinan oligosaccharide.
Aspect H2: The use of aspect H1, or any preceding aspect, further comprising determining whether the subject is at risk of developing the symptoms associated with disrupted circadian rhythm, prior to administering the beta-glucan oligosaccharide and/or the arabinan oligosaccharide. The use of aspect H1, or any preceding aspect may consist essentially of the listed administering step and the listed determining step. The use of aspect H1, or any preceding aspect may consist of the listed administering step and the listed determining step.
Aspect H2a: The use of aspect H2, or any preceding aspect, wherein determining whether the subject is at risk of developing the symptoms comprises assessing the intestinal barrier function of the subject.
Aspect H2b: The use of aspect H2, or any preceding aspect, wherein the subject is assessed to be at risk of developing the symptoms associated with disrupted circadian rhythm.
Aspect H2c: The use of aspect H2, or any preceding aspect, wherein the subject is assessed to be at risk of developing the symptoms associated with disrupted gut barrier function.
Aspect I1: Use of a beta glucan oligosaccharide and/or an arabinan oligosaccharide for the secondary prevention of symptoms associated with disrupted circadian rhythm in a subject.
Aspect J1: Use of a beta glucan oligosaccharide and/or an arabinan oligosaccharide for delaying the progression of symptoms associated with disrupted circadian rhythm in a subject.
Aspect K1: A medicament, for use in reducing the severity of symptoms associated with disrupted circadian rhythm in a subject, the medicament comprising, consisting essentially of, or consisting of an effective amount of a beta-glucan oligosaccharide and/or an arabinan oligosaccharide.
Aspect K2: A medicament, for use in treating symptoms associated with disrupted circadian rhythm in a subject, the medicament comprising, consisting essentially of, or consisting of an effective amount of a beta-glucan oligosaccharide and/or an arabinan oligosaccharide.
Aspect L1: A medicament, for use in the primary prevention of symptoms associated with disrupted circadian rhythm in a subject, the medicament comprising, consisting essentially of, or consisting of an effective amount of a beta-glucan oligosaccharide and/or an arabinan oligosaccharide.
Aspect M1: A medicament, for use in the secondary prevention of symptoms associated with disrupted circadian rhythm in a subject, the medicament comprising, consisting essentially of, or consisting of an effective amount of a beta-glucan oligosaccharide and/or an arabinan oligosaccharide.
Aspect N1: A method of making a medicament for use in reducing the severity of symptoms associated with disrupted circadian rhythm in a subject, comprising, consisting essentially of, or consisting of combining an effective amount of a beta-glucan oligosaccharide and/or an arabinan oligosaccharide with an optional carrier.
Aspect O1: A method of making a medicament for use in treating symptoms associated with disrupted circadian rhythm in a subject, comprising, consisting essentially of, or consisting of combining an effective amount of a beta-glucan oligosaccharide and/or an arabinan oligosaccharide with an optional carrier.
Aspect P1: A method of making a medicament for use in the primary prevention of symptoms associated with disrupted circadian rhythm in a subject, comprising, consisting essentially of, or consisting of combining an effective amount of a beta-glucan oligosaccharide and/or an arabinan oligosaccharide with an optional carrier.
Aspect Q1: A method of making a medicament for use in the secondary prevention of symptoms associated with disrupted circadian rhythm in a subject, comprising, consisting essentially of, or consisting of combining an effective amount of a beta-glucan oligosaccharide and/or an arabinan oligosaccharide with an optional carrier.
Aspect R1: A nutritional formulation or a nutritional supplement comprising, consisting essentially of, or consisting of a beta-glucan oligosaccharide and/or an arabinan oligosaccharide for use in reducing the severity of symptoms associated with disrupted circadian rhythm, or for ameliorating symptoms associated with disrupted circadian rhythm, or for primary prevention of symptoms associated with disrupted circadian rhythm, or for secondary prevention of symptoms associated with disrupted circadian rhythm.
Aspect R2: A nutritional formulation or a nutritional supplement comprising, consisting essentially of, or consisting of a beta-glucan oligosaccharide and/or an arabinan oligosaccharide for use in ameliorating sleep-related symptoms and/or mood related symptoms.
Aspect R3: A nutritional formulation or a nutritional supplement comprising, consisting essentially of, or consisting of a beta-glucan oligosaccharide and/or an arabinan oligosaccharide for use in ameliorating symptoms associated with cognitive functioning and neurocognitive decline.
Aspect R4: A nutritional formulation or a nutritional supplement comprising, consisting essentially of, or consisting of a beta-glucan oligosaccharide and/or an arabinan oligosaccharide for use in ameliorating symptoms including a loss of executive function and/or a loss of memory.
Aspect S1: A pharmaceutical composition comprising a beta-glucan oligosaccharide and/or an arabinan oligosaccharide and optionally comprising a pharmaceutically acceptable carrier or excipient for use in reducing the severity of symptoms associated with disrupted circadian rhythm, or for treating symptoms associated with disrupted circadian rhythm, or for primary prevention of symptoms associated with disrupted circadian rhythm, or for secondary prevention of symptoms associated with disrupted circadian rhythm. The pharmaceutical composition may consist essentially of or consist of a beta-glucan oligosaccharide and/or an arabinan oligosaccharide and a pharmaceutically acceptable carrier or excipient.
Aspect S2: A pharmaceutical composition comprising a beta-glucan oligosaccharide and/or an arabinan oligosaccharide and optionally comprising a pharmaceutically acceptable carrier or excipient for use in treating sleep-related symptoms and/or mood related symptoms. The pharmaceutical composition may consist essentially of or consist of a beta-glucan oligosaccharide and/or an arabinan oligosaccharide and a pharmaceutically acceptable carrier or excipient.
Aspect S3: A pharmaceutical composition comprising a beta-glucan oligosaccharide and/or an arabinan oligosaccharide and optionally comprising a pharmaceutically acceptable carrier or excipient for use in treating symptoms associated with cognitive functioning and neurocognitive decline. The pharmaceutical composition may consist essentially of or consist of a beta-glucan oligosaccharide and/or an arabinan oligosaccharide and a pharmaceutically acceptable carrier or excipient.
Aspect S4: A pharmaceutical composition comprising a beta-glucan oligosaccharide and/or an arabinan oligosaccharide and optionally comprising a pharmaceutically acceptable carrier or excipient for use in treating symptoms including a loss of executive function and/or a loss of memory. The pharmaceutical composition may consist essentially of or consist of a beta-glucan oligosaccharide and/or an arabinan oligosaccharide and a pharmaceutically acceptable carrier or excipient. In this Aspect, the amount of a beta-glucan oligosaccharide and/or an arabinan oligosaccharide in the pharmaceutical composition is an effective amount or combined effective amount of the listed active ingredients.
Aspect T1: A beta-glucan oligosaccharide and/or an arabinan oligosaccharide for use in reducing the severity of symptoms associated with disrupted circadian rhythm, or for ameliorating symptoms associated with disrupted circadian rhythm, or for primary prevention of symptoms associated with disrupted circadian rhythm, or for secondary prevention of symptoms associated with disrupted circadian rhythm.
Aspect T2: A beta-glucan oligosaccharide and/or an arabinan oligosaccharide for use in ameliorating sleep-related symptoms and/or mood related symptoms.
Aspect T3: A beta-glucan oligosaccharide and/or an arabinan oligosaccharide for use in ameliorating symptoms associated with cognitive functioning and neurocognitive decline.
Aspect T4: A beta-glucan oligosaccharide and/or an arabinan oligosaccharide for use in ameliorating symptoms including a loss of executive function and/or a loss of memory.
Aspect U1: A method of prevention, delay of progression or treatment of symptoms associated with disrupted circadian rhythm in a subject, the method comprising consisting essentially of or consisting of administering to the subject an effective amount of a beta glucan oligosaccharide and/or an arabinan oligosaccharide.
Aspect U2: The method of Aspect U1 for treatment of the symptoms associated with disrupted circadian rhythm.
Aspect U3: The method of Aspect U1 for prevention of the symptoms associated with disrupted circadian rhythm. In this aspect, prevention can be primary or secondary prevention of symptoms associated with disrupted circadian rhythm in a subject.
Aspect U4: The method of Aspect U1-U4, further comprising determining whether the subject is at risk of developing the symptoms associated with disrupted circadian rhythm, prior to administering the beta glucan oligosaccharide and/or the arabinan oligosaccharide. In this Aspect, the method can consist essentially or consist of the administration step and the determining steps noted above. In these aspects, determining whether the subject is at risk of developing the symptoms associated with disrupted circadian rhythm comprises, consists essentially or consists of assessing the intestinal barrier function of the subject.
Aspect V1: Use of a beta glucan oligosaccharide and/or an arabinan oligosaccharide in prevention, delay of progression or treatment of symptoms associated with disrupted circadian rhythm. In this Aspect, the prevention can be primary or secondary prevention of symptoms associated with disrupted circadian rhythm. In this Aspect, use can comprise administration and optionally determination steps as noted above. In this Aspect, use can consist essentially of or consist of an administration step as noted above. In this Aspect, use can consist essentially of or consist of administration and determination steps as noted above.
Aspect W1: A beta glucan oligosaccharide and/or an arabinan oligosaccharide for use in the prevention, delay of progression or treatment of symptoms associated with disrupted circadian rhythm. In this Aspect, the prevention can be primary or secondary prevention of symptoms associated with disrupted circadian rhythm. In this Aspect, use can comprise administration and optionally determination steps as noted above. In this Aspect, use can consist essentially of or consist of an administration step as noted above. In this Aspect, use can consist essentially of or consist of administration and determination steps as noted above.
Aspect X1: A nutritional formulation or a nutritional supplement comprising a beta glucan oligosaccharide and/or an arabinan oligosaccharide for use in the prevention, delay of progression or treatment of symptoms associated with disrupted circadian rhythm.
Aspect Z1: A pharmaceutical composition comprising a beta glucan oligosaccharide and/or an arabinan oligosaccharide optionally in combination with a pharmaceutically acceptable carrier or excipient for use in the prevention, delay of progression or treatment of symptoms associated with disrupted circadian rhythm.

In any forgoing Aspect, the symptoms comprise, consist essentially of or consist of sleep-related symptoms.

In any forgoing Aspect, the symptoms comprise, further comprise, consist essentially of or consist of mood-related symptoms.

In any forgoing Aspect, the symptoms comprise, further comprise, consist essentially of or consist of symptoms associated with cognitive functioning and neurocognitive decline.

In any forgoing Aspect, the symptoms comprise, further comprise, consist essentially of or consist of a loss of executive function and/or a loss of memory.

In any forgoing Aspect, the amount of the beta glucan oligosaccharide and/or the arabinan oligosaccharide administered is an amount or combined amount effective for treatment.

In any forgoing Aspect, the amount of the beta glucan oligosaccharide and/or the arabinan oligosaccharide administered is an amount or combined amount effective for treatment ranges from about 0.5 g to about 50 g per day.

In any forgoing Aspect, at least 50% of the mass of the beta glucan oligosaccharide or the arabinan oligosaccharide has a molecular weight of 100 kDa or less.

In any forgoing Aspect, the beta-glucan oligosaccharide and/or the arabinan oligosaccharide is generated by reacting polysaccharides in a reaction mixture with a Fenton's reagent, having a peroxide agent and metal ions, to provide treated polysaccharides; and cleaving the treated polysaccharides with a base to generate a mixture of oligosaccharides.

In any forgoing Aspect, the beta glucan oligosaccharide and/or the arabinan oligosaccharide is selected from the group consisting of CLX115, CLX122, CLX115Cu, CLX122DSF, CLX112, or any combination thereof.

In any forgoing aspect, the beta glucan oligosaccharide and/or the arabinan oligosaccharide is selected from the group consisting of CLX115Cu or any combination thereof with CLX115, CLX122, CLX122DSF, or CLX112.

In any forgoing Aspect, the beta glucan oligosaccharide is CLX115Cu.

In any forgoing Aspect, the beta glucan oligosaccharide is CLX115.

In any forgoing Aspect, the beta glucan oligosaccharide is CLX112.

In any forgoing Aspect, the arabinan oligosaccharide is CLX122.

In any forgoing Aspect, the arabinan oligosaccharide is CLX122DSF.

In any forgoing Aspect, for primary prevention of symptoms associated with disrupted circadian rhythm in a subject, the subject or patient is optionally assessed for the risk of developing the symptoms associated with disrupted circadian rhythm, prior to administering the beta-glucan oligosaccharide and/or the arabinan oligosaccharide.

In any forgoing Aspect, for primary prevention of symptoms associated with disrupted circadian rhythm in a subject, the subject or patient is optionally assessed for the risk of developing a disrupted gut barrier.

In any forgoing Aspect, for secondary prevention of symptoms associated with disrupted circadian rhythm in a subject, the subject or patient is optionally assessed for the risk of developing the symptoms associated with disrupted circadian rhythm, prior to administering the beta-glucan oligosaccharide and/or the arabinan oligosaccharide.

In any forgoing Aspect, for secondary prevention of symptoms associated with disrupted circadian rhythm in a subject, the subject or patient is optionally assessed for the risk of developing a disrupted gut barrier.

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference). All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (e.g., to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, example embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be apparent to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

The invention illustratively described herein suitably may be practiced in the absence of any element or elements, limitation or limitations which is not specifically disclosed herein.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be examples, as it is known that one of ordinary skill in the art can name the same compounds differently.

Certain molecules disclosed herein may contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every device, system, formulation, combination of components, or method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### THE EXAMPLES

The examples described below are for illustration purposes only. The invention is not limited in scope to embodiments described in the examples.

### Example 1 Effect of Microbial Metabolites on Intestinal Cell Permeability Using an In Vitro Caco-2 Cell Model

To gain insight into the effect of CLX115 and CLX122 on gut barrier permeability, we evaluated the effect of fecal fermentation supernatants on restoring disrupted Caco-2 gut epithelial cells.

Static fecal fermentations with a fecal sample from two healthy donors, donor A and donor B, were conducted in 1 mL volumes, under anaerobic conditions (Anaerobic Chamber Vinyl Type B), using a mix of gas (carbon dioxide 5%, hydrogen 5%, nitrogen balance). CLX115 and CLX122 were used at a concentration of 0.6% w/v. An untreated control (fecal fermentation without added oligosaccharide) was run. Fermentation medium was optimized to support diverse microbial taxa and control pH within the range of the colon physiological conditions, containing mineral and vitamin solution, CaCl₂) (10 mg/mL) and basic fermentation medium as described by MacFalane G T et al (1989). After 20 hours of fermentation, supernatants were collected and stored at −20° C.

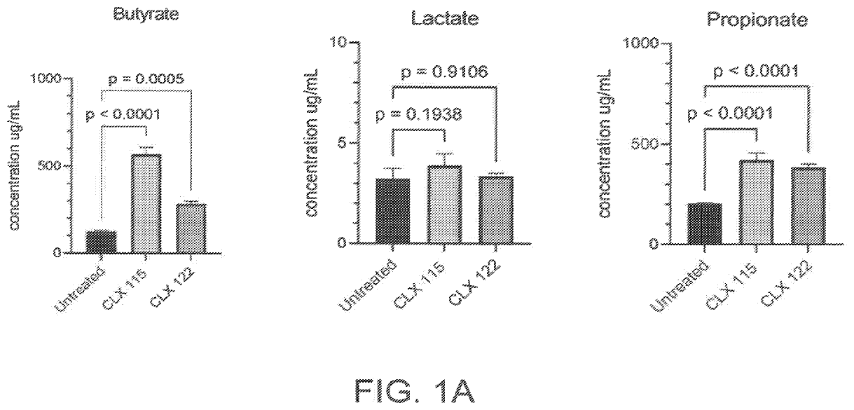

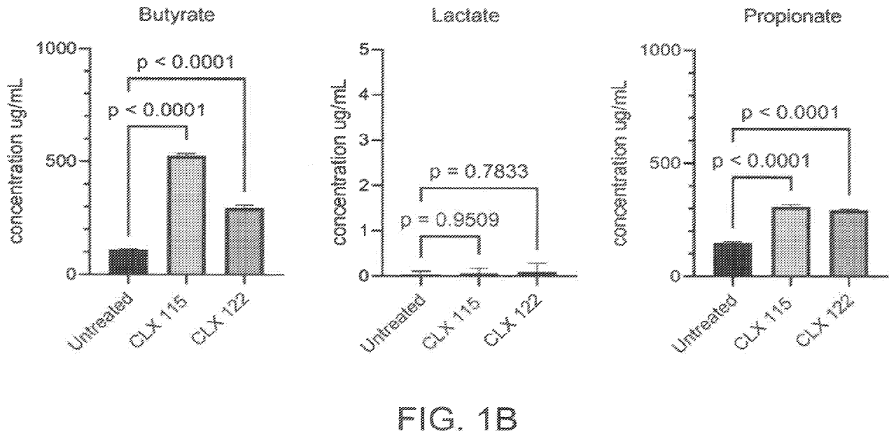

Short chain fatty acids (SCFAs), particularly acetate, propionate, and butyrate, are mainly produced by anaerobic fermentation of gut microbes. SCFAs have demonstrated physiologically beneficial effects, like restoring gut barrier function. To verify the presence of these microbial metabolites in the tested supernatants, saved samples from CLX115 and CLX122 fermentations as well as the untreated control were analyzed for SCFA content. Supernatants were derivatized with 2-nitrophenylhydrazine (2-NPH) for LC-MS/QqQ analysis. The results shown in FIGS. 1A and 1B indicate that microbial communities of donor A and donor B, respectively, can produce butyric acid and propionate as products of fermentation of CLX115 and CLX122. These values were significantly higher than for the untreated samples (fermented without addition of oligosaccharides). Data for each SCFA was analyzed separately. Ordinary one-way ANOVAs were run across treatments. This was followed by a Dunnett's multiple comparison test of each CLX glycan composition against the untreated control.

Next, using the same fecal fermentation supernatants, we evaluated their effect in restoring permeability of intestinal epithelial-like cells (Caco-2 cells). Tight junction proteins keep Caco-2 adjacent cells together, forming an impermeable barrier to macromolecules, and the “tightness” of these junctions can be measured as transepithelial electrical resistance (TEER), with a high TEER corresponding to a tighter barrier. Caco-2 cells were cultured at 37° C. in a humidified atmosphere of C02/air in minimum essential medium (MEM) supplemented with 10% (v/v) fetal bovine serum and antibiotics (50 U/ml penicillin and 50 μg/ml streptomycin). For these experiments, cells were seeded onto trans-well plates and grown for 14-18 days until differentiated, replacing the media every 3 days. For the treatments, supernatants from Donor A and Donor B fermentation experiments were centrifuged and sterile filtered to remove cells and diluted 1:2 in MEM. For the positive control, 5 mM sodium butyrate (Na-Bu) was prepared in MEM. The control and control-leaky samples were treated with MEM alone. Cells were pre-incubated with treatment in the apical chamber for 1 hour prior to disruption, after which 10 ng/mL TNF-α and IFN-γ were added to the basolateral chamber. TEER measurements were taken before treatments, at 0 hours, and 24 hours after disruption, and results were expressed as % change in TEER value between 24 and 0 hours. Ordinary one-way ANOVA was run first to determine whether treatments were significantly different, followed by Dunnett's multiple comparisons test comparing the mean of each treatment to the untreated control. We observed that supernatants from CLX115 and CLX122 fecal fermentation were able to significantly increase TEER of the Caco-2 cells compared to treatment with supernatants from untreated fecal fermentation, for both Donor A and Donor B.

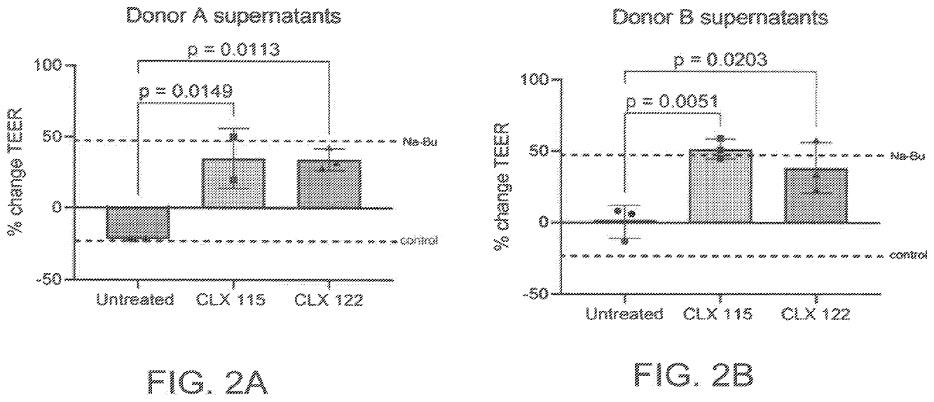

These results shown in FIGS. 2A and 2B indicate that supernatants from static fecal fermentations performed with CLX115 and CLX122 can restore damaged Caco2-cells, thus, being candidates for restoring disrupted intestinal cell barrier, thus being a candidate for circadian rhythm regulation.

### Example 2—Effect of Microbial Metabolites on Gut Barrier Integrity Using an In Vitro Caco-2/THP-1 Leaky Gut Model

To gain further insight on the effect of CLX115 and CLX122 on gut barrier integrity, we tested the effect of supernatants coming from a more complex fecal fermentation model in a co-culture cell assay. The fermentation of the oligosaccharides by the fecal community was done in continuous mode for 3 weeks. This system is called the Simulator of the Human Intestinal Microbial Ecosystem (SHIME) and allows simulation of physiology and microbiology of the gastrointestinal (GI) tract. [Molly K. et al., 1993] The typical reactor setup of the SHIME consists of a succession of three reactors, the first one stimulates the different steps in food uptake and digestion (representing stomach and small intestine). The other two reactors simulate the large intestine (proximal and distal colon) and are inoculated with fecal samples from a healthy donor. The experiment was divided into three different stages: control stage (2 weeks, stabilization of reactors and fecal sample, being the baseline microbial community and activity), treatment stage (3 weeks, CLX115 or CLX122) was added three times a day with the feed to simulate repeated intake, revealing the effects of the oligos) and wash out (2 weeks). Samples were taken at the end of the end of control and treatment phase.

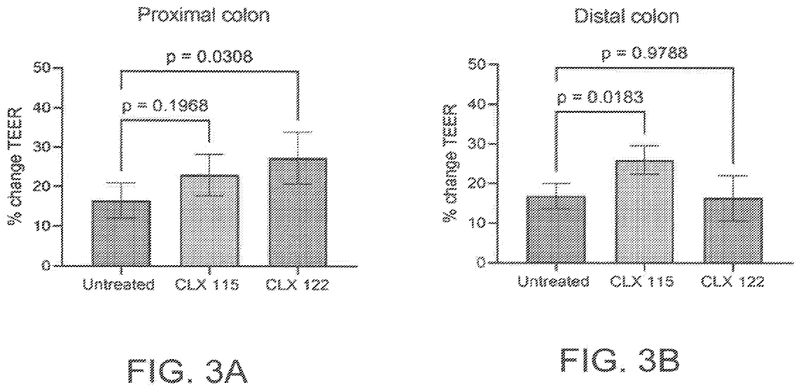

Next, we evaluated the effect of these supernatants in a Caco-2/THP-1 co-culture in vitro model (Possemiers et al., 2013). By adding phorbol 12-myristate 13-acetate (PMA) treatment, THP-1 cells acquire morphological features characteristic of macrophage, being able to adhere to a support, and secrete cytokines into the supernatants. When Caco-2 cells are seeded onto trans-well plates and placed on top of PMA-activated THP-1 cells, their monolayer becomes disrupted, and this disruption can be measured as a decrease in TEER value. Briefly, before setting up the co-culture, the TEER of the Caco-2 monolayers was measured (0 hour time point). The TEER of an empty insert was subtracted from all readings to account for the residual electrical resistance of an insert. Then, the Caco-2-bearing inserts were placed on top of the PMA-differentiated THP1-Blue™ cells. The apical compartment (containing the Caco-2 cells) was filled with sterile-filtered (0.22 μm) colonic suspensions. Cells were also treated apically with Na-Bu (sodium butyrate, Sigma-Aldrich) as positive control. The basolateral compartment (containing the THP1-Blue™ cells) was filled with Caco-2 complete medium. Cells were also exposed to Caco-2 complete medium in both chambers as control. Cells were treated for 24 h, after which the TEER was measured (24 hour time point). After subtracting the TEER of the empty insert, each 24 hour value was normalized to its own 0 hour value (to account for the differences in initial TEER of the different inserts) and is presented as percentage of initial value. For each compartment, the proximal and distal colons, a one-way ANOVA was performed followed by Dunnett's multiple comparisons test of each oligosaccharide treated group vs untreated control. P-values are indicated in FIGS. 3A and 3B.

All SHIME suspensions were able to maintain and even further increase TEER of the Caco-2 cells compared to their initial values, but differences in the oligosaccharides are noted in the location of their effect. CLX122 treatment significantly increased TEER value in the proximal colon compared to its control, while CLX115 significantly increased TEER in the distal colon (FIGS. 3A and 3B). Thus, these results indicate that fermentation of CLX115 and CLX122 show additional protective effect on inflammation-induced intestinal epithelial barrier disruption in the proximal and distal colon of the in vitro colonic SHIME fermentations.

These results indicate that supernatants from semi-continuous fecal fermentations performed in a SHIME model with CLX115 and CLX122 can restore damaged Caco2-cells, thus, being candidates for circadian rhythm regulation.

### Example 3—In Vitro Effect of Oligosaccharides on Inflammation

Decrease in the proinflammatory cytokines like TNF-α and IL-1β, have been shown to be associated with improvement in cognitive function (Noble et al. 2017). TNF-α and IL-1β will induce the production of chemokines (e.g., IL-8 and chemokine CXCL-10) and adhesion molecules necessary for reactive oxygen species (ROS) production. ROS production seals breaches in the epithelial wall, but this may cause inflammation, leading to production of anti-inflammatory cytokines, like IL-6 and IL-10 (Koelink et al. 2020). Measurements of the mentioned cytokines and chemokines are used to evaluate the impact of a potential treatment in neuroinflammation and HPA axis related diseases.

To gain insight into the effect of CLX115 and CLX122 after fermentation on immune modulation at the gut barrier level, we performed an additional assay with the Caco-2/THP-1 in vitro co-culture model from Example 2. After TEER reading, the basolateral supernatant was discarded, and cells were stimulated at the basolateral side with Caco-2 complete medium containing lipopolysaccharide (LPS). After LPS stimulation, the basolateral supernatants were collected for cytokine measurement by Luminex multiplex (Thermo Fisher Scientific). All treatments were done in triplicate and cells were incubated at 37° C. in a humidified atmosphere of air/CO₂ (95:5, v/v). Different immune markers were evaluated, including effect on pro-inflammatory chemokines CXCL10, IL-8 and MCP-1, anti-inflammatory cytokines IL-10 and IL-6, and pro-inflammatory cytokines IL-1β and TNF-α. Unpaired t-tests were performed for each immune marker comparing the treated samples to the control.

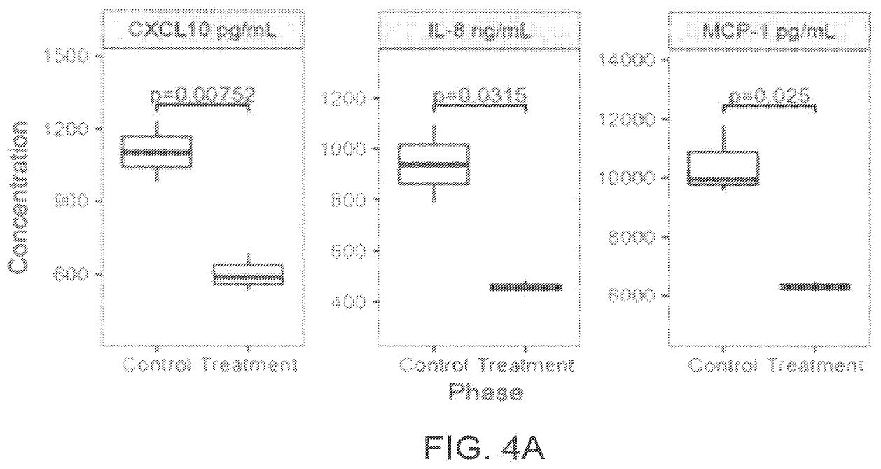

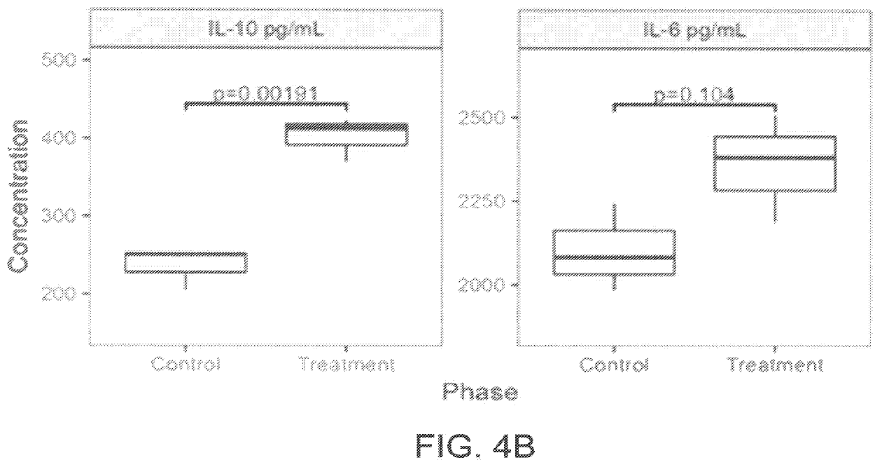

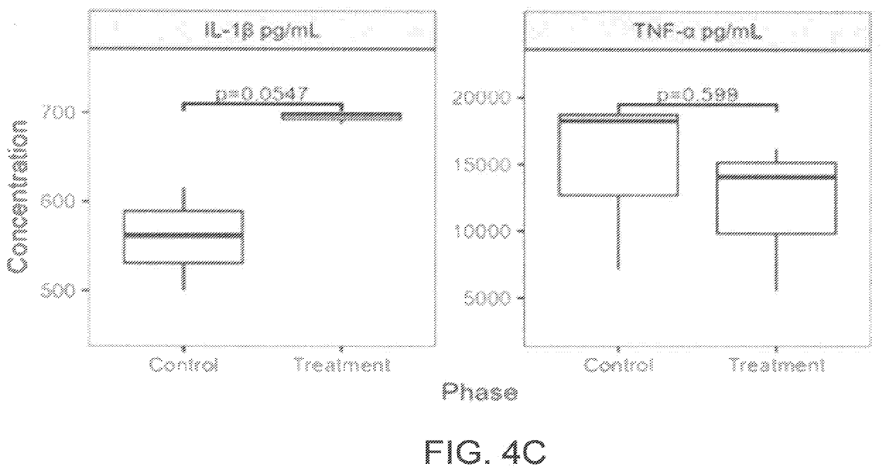

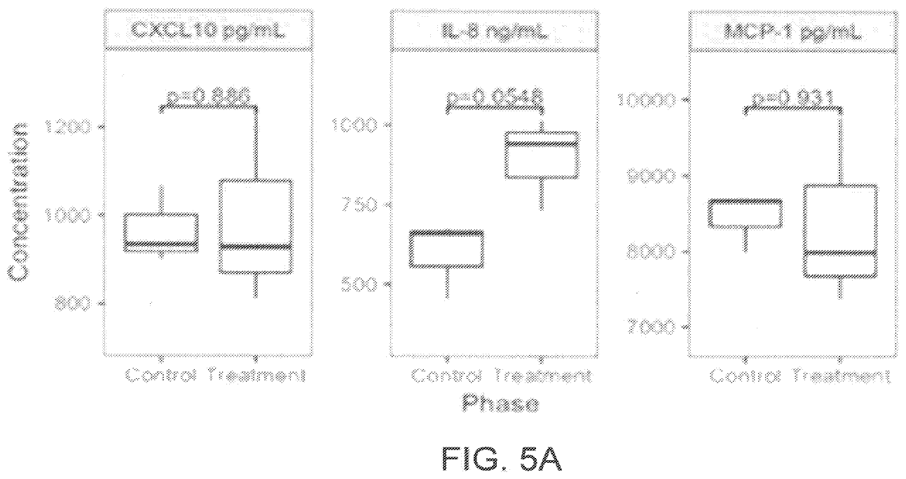

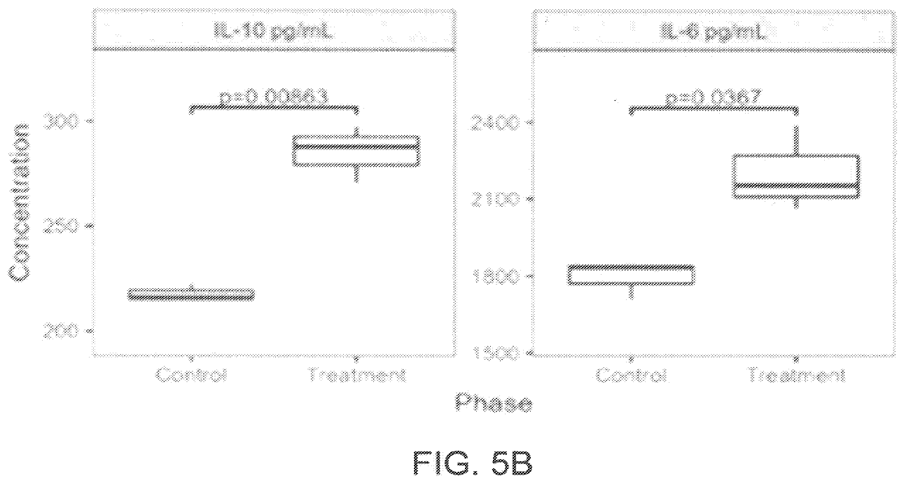

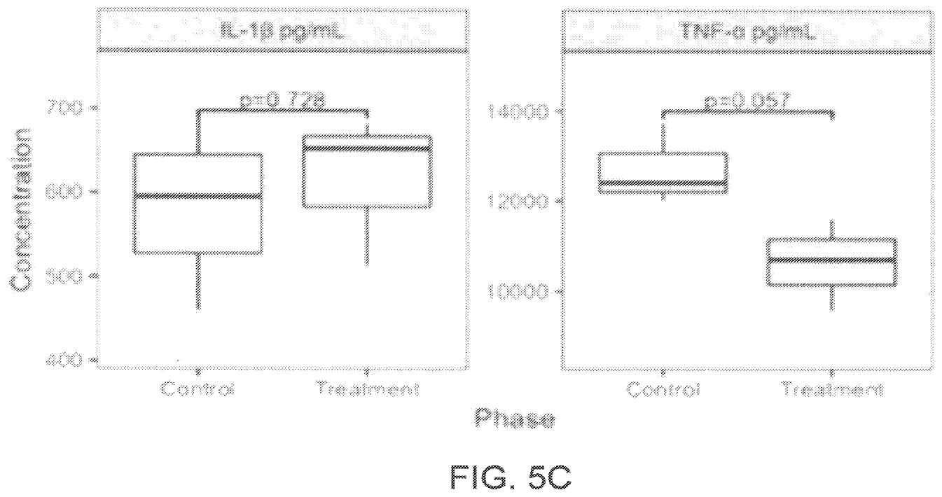

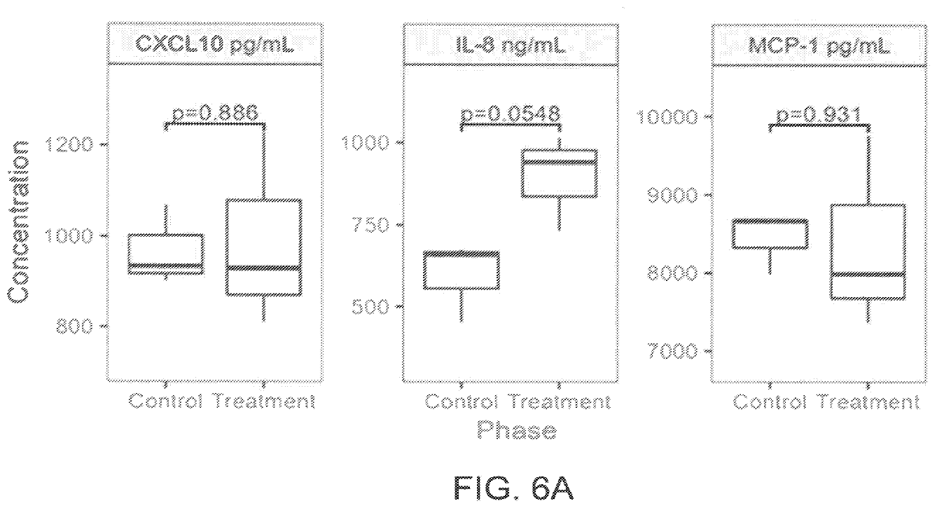

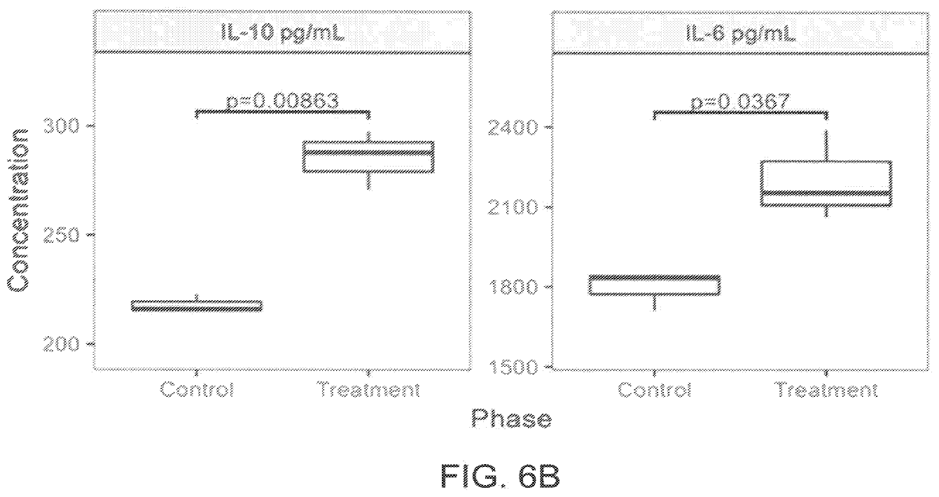

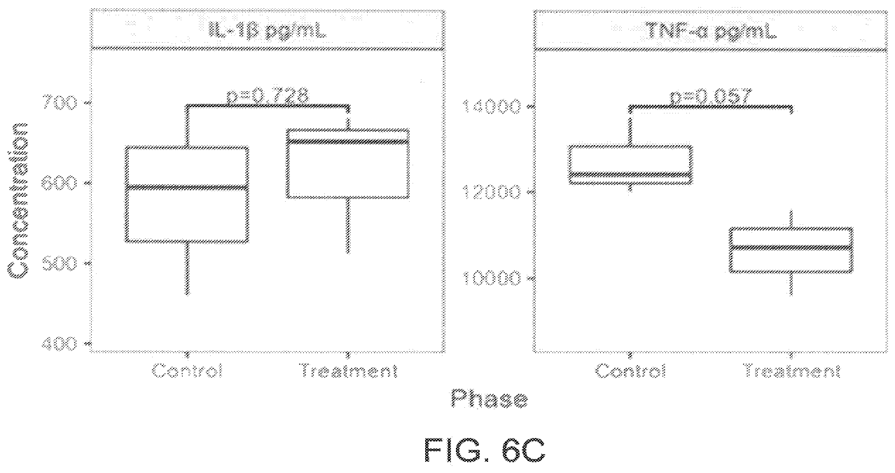

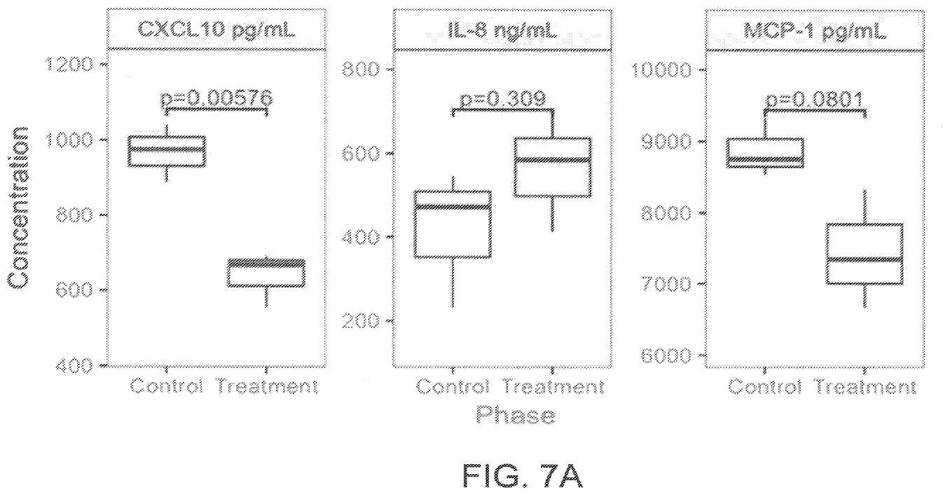

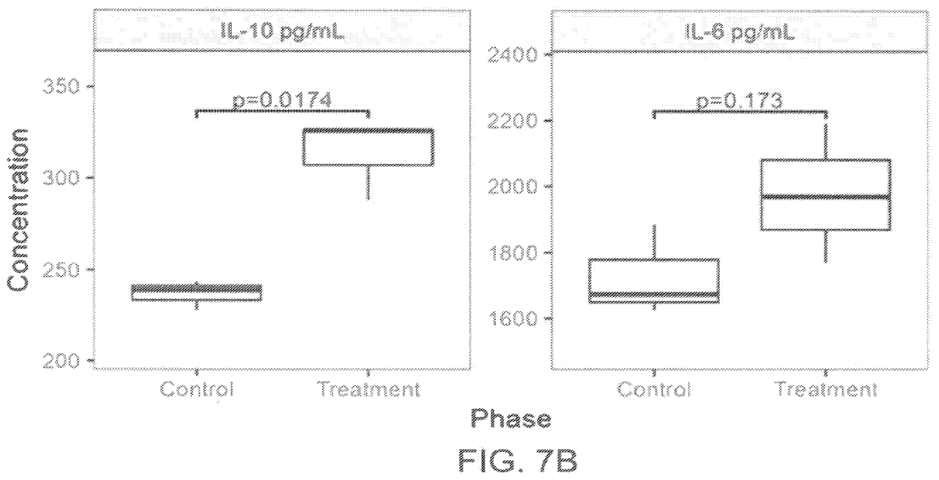

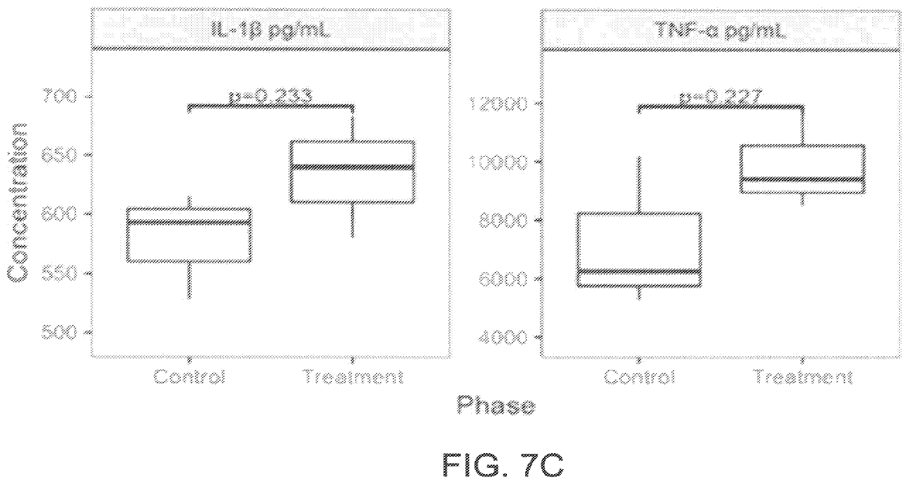

Results show that while colonic fermentation of CLX115 and CLX112 impact inflammatory markers, the effect of each oligo differs in the location (proximal colon vs distal colon) and severity. Fermentation of CLX115 showed a pronounced anti-inflammatory effect with samples from proximal colon, by significantly reducing the secretion of pro-inflammatory cytokines IL-1β and TNF-α, and significantly decreasing the production of CXCL10 and MCP-1. The results are shown in FIGS. 5A-5C. If samples from the distal colon are used in the assay, we observe that CLX115 fermentation also significantly decreases the chemokine CXCL10 and a significant increase of the anti-inflammatory cytokine IL-10 is observed (FIGS. 4A-4C). Colonic fermentation of CLX122 had a milder anti-inflammatory effect, with a significant decrease of CXCL10 and an increase of IL-10 with samples from proximal colon (FIGS. 7A-7C), and a significant increase of anti-inflammatory cytokines IL-10 and IL-6 with distal colon samples (FIGS. 6A-6C).

These results indicate that supernatants from semi-continuous fecal fermentations performed in a SHIME model with CLX115 and CLX122 can decrease inflammatory markers and increase anti-inflammatory cytokines in a co-culture Caco2/TH1-P model, thus, being candidates for circadian rhythm regulation.

### Example 4—In Vitro Production of GABA by Microbial Fecal Communities

To gain insight if the neurotransmitter GABA can be produced by microbial communities in the presence of CLX115 and CLX122, we performed in vitro static fecal fermentation with multiple donors (n=20), as described in Example 1. Next, after 24 h of fermentation, when the microbial community has been shifted by the carbohydrate source, the media were supplemented with a mix of amino acids that contains 50 ul/ml of glutamate, precursor of GABA. Supernatants were collected after 30 min fermentation, and levels of glutamate and GABA were determined by LC-MS/QTOF analysis. Metabolites were analyzed with a 1290 Infinity II LC (Agilent Technologies, Santa Clara, CA) equipped with a HILIC column (InfinityLab Poroshell 120 HILIC-Z, 2.1×150 mm; Agilent Technologies, Santa Clara, CA) and 6530 LC-MS QTOF (Agilent Technologies, Santa Clara, CA). LC separation was performed with 10% 200 mM ammonium formate with 0.10% Formic Acid plus 90% Water (solvent A) and 10% 200 mM ammonium formate with 0.1% Formic Acid+90% Acetonitrile (solvent B). The MS conditions were set to positive mode with a scan range set at m/z 50-1700 at 1 spectra/see scan rate. Peak area was quantitated using Agilent Quantitative Analysis software and areas were normalized to internal standards for quantitation.

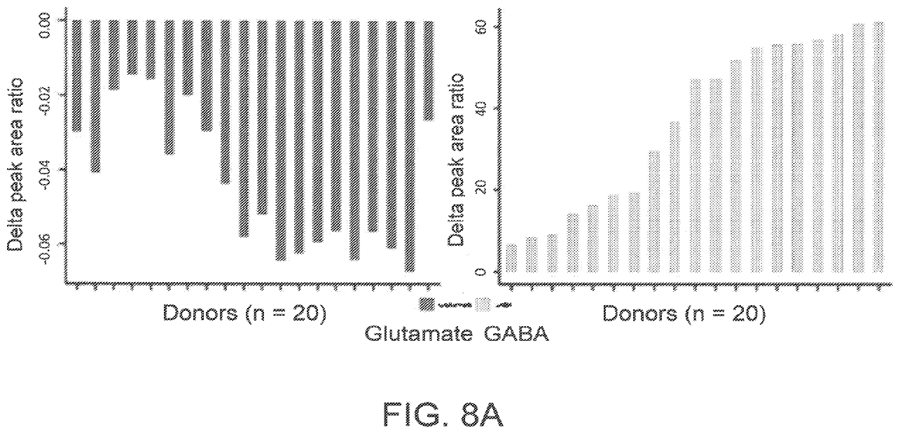

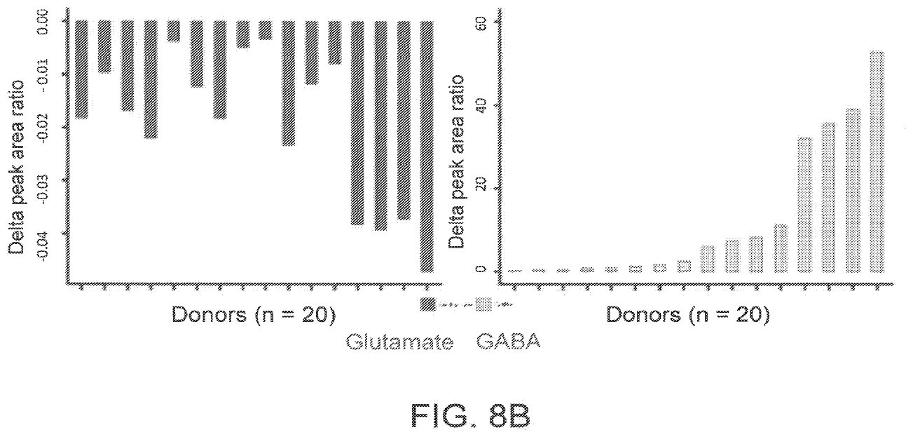

Results indicate that fecal fermentation of CLX115 and CLX122 shift microbial communities from different donors to an optimal composition that allows conversion of glutamate into the neurotransmitter GABA (FIG. 8A and FIG. 8B). GABA has been shown to interact with pain receptors and it functions as the primary inhibitory neurotransmitter for the central nervous system, thus being a candidates for circadian rhythm regulation.

### Example 5—In Vitro Ammonia Production by Fecal Microbiome

To gain insight into the effect of CLX115 on ammonia production, we performed fecal fermentations with multiple donor samples.

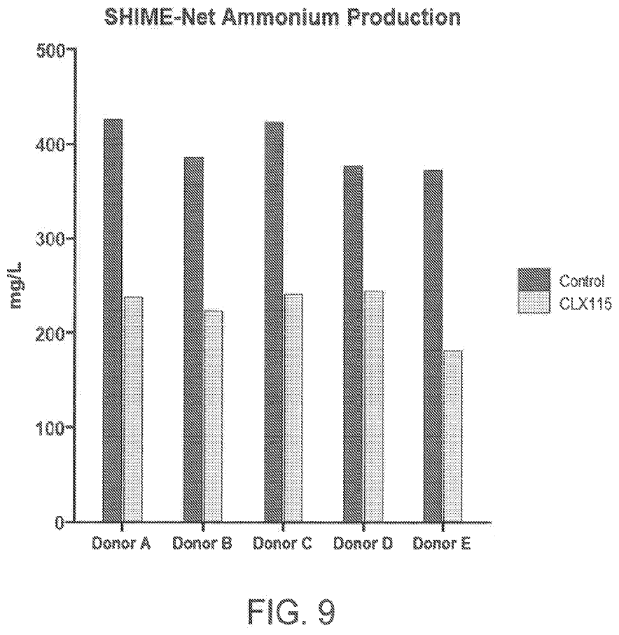

Static fecal fermentations with a fecal sample from five healthy donors, were conducted in 1 ml volumes, under anaerobic conditions (Anaerobic Chamber Vinyl Type B), using a mix of gas (carbon dioxide 5%, hydrogen 5%, nitrogen balance) during 48 h. CLX115 was added at a concentration of 3.5 mg/ml. An untreated control (fecal fermentation without added oligosaccharide) was run. Fermentation media was optimized to support diverse microbial taxa and control pH within the range of the colon physiological conditions. After 48 hours of fermentation, supernatants were collected and stored at −20° C. Samples were then analyzed using an ammonia colorimetric assay kit II (Biovision, CA). All the untreated control incubations resulted in an ammonium production between 372-426 mg/L. Supplementation with CLX115 resulted in reduced ammonium levels for all donors tested (FIG. 9).

Results indicate that fecal fermentation of CLX115 shift microbial communities to reduced levels of ammonia production. Neurotoxic ammonia produced by the gut microbiota may pass through the vagus nerve into the central nervous system, thereby affecting brain function, stress responses, and sleep structure. Thus, CLX115 is a candidate for restoring circadian rhythm regulation.

### Example 6—Nutritional Formula

A powdered nutritional formula is prepared from whey protein concentrate (45%), skimmed milk powder (40%), maltodextrin, inulin, oligosaccharide CLX122, vitamins, minerals, and flavors. The formula has the following composition:

| Nutrient | Per 100 g powder |
|---|---|
| Energy (kcal) | 372 |
| Protein (g) | 45 |
| Lipid (g) | 2.8 |
| Carbohydrate (g) | 41.7 |
| Fibre (g) | 5 |
| CLX122 (g) | 5 |

### Example 7: Optimized Conditions for Copper-Based Fenton Depolymerization of Beta Glucan and Arabinan

The use of iron-based and copper-based Fenton depolymerization has been demonstrated in several previous publications (WO2021097138A1, WO2018236917A1, WO2020247389A1, WO2022241163A1 and WO2023220318). However, a thorough optimization of copper-based Fenton depolymerization has never been demonstrated. Here, we show a set of optimized parameters and provide instructions for the copper-based Fenton depolymerization of beta glucan and arabinan, particularly from cereals or legumes.

A solution containing 4% hydrogen peroxide with 43.4 mM, pH 5.5 ammonium acetate buffer is heated to 55° C. Beta Glucan or arabinan (or other) polysaccharides are stirred in gradually to a final concentration of 10%. To initiate the first reaction step, Copper (II) sulfate is added to a final concentration of 0.75 mM. The reaction is allowed to proceed for 2 hours at 55° C. then cooled to below 15° C. Next, to initiate the second reaction step, concentrated ammonium hydroxide is added to a final concentration of 0.67 M. The reaction is allowed to stir at 45° C. for 2 hours. The reaction is filtered by vacuum with a GD120 filter and Buchner funnel, then treated with MB10 resin (10% w/v) until the electrical conductivity is below a threshold of 100 μS/cm. Resin is removed by vacuum filtration using a glass-fritted funnel and the filtrate is frozen, then lyophilized to dryness. The lyophilized product mixture is then solubilized in minimal ultra-pure H₂O after which a volume of 200 proof food-grade ethanol is added to create a 60% ethanol solution. The solution is then separated by centrifugation (4700 RPM, 15 min, −10° C.). The supernatant is carried forward while the pellet is once again solubilize din minimal ultra-pure H₂O after which a volume of 200 proof food-grade ethanol is added to create a 60% ethanol solution. The solution is then separated by centrifugation (4700 RPM, 15 min, −10° C.). The cumulative supernatant volume is reduced by rotary evaporation, then lyophilized to yield a fluffy, white, crystalline solid.

### Example 8. Optimized Conditions for Iron-Based Fenton Depolymerization

The use of iron-based and copper-based Fenton depolymerization has been demonstrated in several previous publications (WO2021097138A1, WO2018236917A1, WO2020247389A1 WO2022241163A1 and WO2023220318). Here, we show a set of optimized parameters for the iron-based Fenton depolymerization of beta glucan and arabinan, particularly from cereals and legumes.

A solution containing 7% hydrogen peroxide with 43.4 mM, pH 5.5 ammonium acetate buffer is heated to 55° C. Beta Glucan or arabinan (or other) polysaccharides are stirred in gradually to a final concentration of 5%. To initiate the first reaction step, Iron (II) sulfate is added to a final concentration of 1.15 mM. The reaction is allowed to proceed for 2 hours at 55° C. then cooled to below 15° C. Next, to initiate the second reaction step, concentrated ammonium hydroxide is added to a final concentration of 0.39 M. The reaction is allowed to stir at 45° C. for 2 hours. The reaction is filtered by vacuum with a GD120 filter and Buchner funnel, then treated with MB10 resin (10% w/v) until the electrical conductivity is below a threshold of 100 μS/cm. Resin is removed by vacuum filtration using a glass-fritted funnel and the filtrate is frozen, then lyophilized to dryness. The lyophilized product mixture is then solubilized in minimal ultra-pure H₂O after which a volume of 200 proof food-grade ethanol is added to create a 60% ethanol solution. The solution is then separated by centrifugation (4700 RPM, 15 min, −10° C.). The supernatant is carried forward while the pellet is once again solubilized in minimal ultra-pure H₂O after which a volume of 200 proof food-grade ethanol is added to create a 60% ethanol solution. The solution is then separated by centrifugation (4700 RPM, 15 min, −10° C.). The cumulative supernatant volume is reduced by rotary evaporation, then lyophilized to yield a fluffy, white, crystalline solid.

### Example 9—Nutritional Product in Capsule Form

A capsule is prepared by filling about 1 g of CLX122DSF into a 000 gelatine capsule using a filing machine. The capsules are then closed. The CLX122DSF is in free flowing, powder form.

### Example 10—Nutritional Product in Stick Pack Form

Oligosaccharide 115Cu is dissolved in water and then dried and granulated in a fluidized bed drier. The granulated oligosaccharide is then filled into 5 g stick packs using a 10-lane vertical form/fill/seal filling machine. The packaging material is a 3-layer film made up of polyethylene terephthalate (PET), aluminum and linear low-density polyethylene (LLPDE) with a laser cut for easy opening. The stick packs are then packed into secondary packaging each containing 28 stick packs.

### REFERENCES

1. Amicucci, M. J., Galermo, A. G., Nandita, E., Vo. T-T. T., Liu, Y., Lee. M., Xu, G., Lebrilla, C. B. (2019) “A rapid-throughput adaptable method for determining the monosaccharide composition of polysaccharides.” International Journal of Mass Spectrometry 438: 22-28.
2. Amicucci, M. J., Galermo, A. G., Guerrero, A., Treves, G., Nandita, E., Kailemia, M. J., Higdon, S. M., Pozzo, T., Labavitch, J. M., Bennett, A. B., Lebrilla, C. B. (2019) “Strategy for Structural Elucidation of Polysaccharides: Elucidation of a Maize Mucilage that Harbors Diazotrophic Bacteria,” Analytical Chemistry 91:7254-7265.
3. Amicucci, M. J., Nandita, E., Galermo, A. G., Castillo, J. J., Chen, S., Park, D., Smilowitz, J. T., German, J. B., Mills, D. A., Lebrilla, C. B. (2020) “A nonenzymatic method for cleaving polysaccharides to yield oligosaccharides for structural analysis.” Nature communications 11: 3963 (12 pages).
4. Fifel. K., Videnovic, A. (2012) “Circadian and sleep dysfunctions in neurodegenerative disorders—An update.” Front. Neurosci. 14 (627330) (7 pages).
5. Galermo, A. G., Nandita, E., Barboza, M., Amicucci, M. J., Vo. T-T. T., Lebrilla, C. B. (2018) “Liquid chromatography-tandem mass spectrometry approach for determining glycosidic linkages.” Analytical Chemistry 90(21): 13073-13080.
6. Galermo, A. G., Nandita, E., Castillo, J. J., Amicucci, M. J., Lebrilla, C. B. (2019) “Development of an Extensive Linkage Library for Characterization of Carbohydrates,” Analytical Chemistry 91(20): 130222-13031.
7. Koelink, P. J., Bloemendaal, F. M., Li, B., Westera. L., Vogels, E. W. M., van Roest, M., Gloudemans, A. K., van't Wout, A. B., Korf, H., Vermeire, S., te Velde, A. A., Ponsioen, C. Y., D'Haens. G. R. A. M., Verbeek, J. S., Geiger, T. L., Wildenberg, M. E., van den Brink, G. R. (2020) “Anti-TNF therapy in IBD exerts its therapeutic effect through macrophage IL-10 signalling,” Gut 69:1053-1063.
8. Macfarlane, G. T., Cummings, J. H., Macfarlane, S., Gibson, G. R. (1989) “Influence of retention time on degradation of pancreatic enzymes by human colonic bacteria grown in a 3-stage continuous culture system.” J Appl Bacteriol. November 67(5):520-7.
9. Molly, K., Vande Woestyne, M., Verstraete, W. (1993) “Development of a 5-step multichamber reactor as a simulation of the human intestinal microbial ecosystem.” Applied Microbiology and Biotechnology 39: 254.
10. Noble, E. N., Hsu, T. M, Kanoski, S. E. (2017) “Gut to brain dysbiosis: Mechanisms linking western diet consumption, the microbiome, and cognitive impairment.” Front. Behav. Neurosci. 11(9) (10 pages).
11. Possemiers, S., Pinherio, I., Verhelst, A., Van den Abbeele, P., Maignien, L., Laukens, D., Reeves, S. G., Robinson. L. E., Rass, T., Schneider, Y-J., Van de Wiele, T., Marzorati, M. (2013) “A dried yeast fermentate selectively modulates both the luminal and mucosal gut microbiota and protects against inflammation, as studied in an integrated in vitro approach.” J Agric. Food. Chem. 61:9380.
12. Teichman, E. M., O'Riordan K. J., Gahan, C. G. M., Dinan, T. G., Cryan, J. F. When rhythms meet the blues: circadian interactions with the microbiota-gut-brain axis. Cell Metabolism, 2020, 31:448

We claim:

1. A method of prevention, delay of progression or treatment of symptoms associated with disrupted circadian rhythm in a subject, the method comprising administering to the subject an effective amount of a beta glucan oligosaccharide and/or an arabinan oligosaccharide.

2. The method of claim 1 for treatment of the symptoms associated with disrupted circadian rhythm.

3. The method of claim 1 for prevention of the symptoms associated with disrupted circadian rhythm.

4. The method of claim 3 for the primary or secondary prevention of symptoms associated with disrupted circadian rhythm in a subject.

5. The method of any one of claims 1-4, further comprising determining whether the subject is at risk of developing the symptoms associated with disrupted circadian rhythm, prior to administering the beta glucan oligosaccharide and/or the arabinan oligosaccharide.

6. The method of claim 5, wherein determining whether the subject is at risk of developing the symptoms associated with disrupted circadian rhythm comprises assessing the intestinal barrier function of the subject.

7. Use of a beta glucan oligosaccharide and/or an arabinan oligosaccharide in prevention, delay of progression or treatment of symptoms associated with disrupted circadian rhythm.

8. The use of claim 7 in primary or secondary prevention of symptoms associated with disrupted circadian rhythm.

9. A beta glucan oligosaccharide and/or an arabinan oligosaccharide for use in the prevention, delay of progression or treatment of symptoms associated with disrupted circadian rhythm.

10. The oligosaccharide for use of claim 9, wherein prevention of symptoms is primary or secondary prevention of symptoms associated with disrupted circadian rhythm.

11. The method, use or oligosaccharide for use of any one of claims 1 to 10, wherein the symptoms comprise sleep-related symptoms.

12. The method, use or oligosaccharide for use of any one of claims 1 to 11, wherein the symptoms comprise or further comprise mood-related symptoms.

13. The method, use or oligosaccharide for use of any one of claims 1 to 12, wherein the symptoms comprise or further comprise symptoms associated with cognitive functioning and neurocognitive decline.

14. The method, use or oligosaccharide for use of any one of claims 1-13, wherein the symptoms comprise or further comprise a loss of executive function and/or a loss of memory.

15. The method, use or oligosaccharide for use of any one of claims 1 to 14, wherein the amount of the beta glucan oligosaccharide and/or the arabinan oligosaccharide effective for treatment, delay or prevention ranges from about 0.5 g to about 50 g per day.

16. The method, use or oligosaccharide for use of any one of claims 1-15, wherein at least 50% of the mass of the beta glucan oligosaccharide or the arabinan oligosaccharide has a molecular weight of 100 kDa or less.

17. The method, use or oligosaccharide for use of any one of claims 1-16, wherein the beta-glucan oligosaccharide and/or the arabinan oligosaccharide is generated by reacting polysaccharides in a reaction mixture with a Fenton's reagent, having a peroxide agent and metal ions, to provide treated polysaccharides; and cleaving the treated polysaccharides with a base to generate a mixture of oligosaccharides.

18. A nutritional formulation or a nutritional supplement comprising a beta glucan oligosaccharide and/or an arabinan oligosaccharide for use in the prevention, delay of progression or treatment of symptoms associated with disrupted circadian rhythm.

19. A pharmaceutical composition comprising a beta glucan oligosaccharide and/or an arabinan oligosaccharide optionally in combination with a pharmaceutically acceptable carrier or excipient for use in the prevention, delay of progression or treatment of symptoms associated with disrupted circadian rhythm.

20. The nutritional formulation or nutritional supplement of claim 18 or the pharmaceutical composition of claim 19, wherein at least 50% of the mass of the beta glucan oligosaccharide or the mass of the arabinan oligosaccharide has a molecular weight of 100 kDa or less.

21. The nutritional formulation or nutritional supplement of claim 18 or 20 or the pharmaceutical composition of claim 19 or 20, wherein the beta-glucan oligosaccharide and/or the arabinan oligosaccharide is generated by reacting polysaccharides in a reaction mixture with a Fenton's reagent, having a peroxide agent and metal ions, to provide treated polysaccharides; and cleaving the treated polysaccharides with a base to generate a mixture of oligosaccharides.
